(19) United States
(12) Patent Application Publication
(10) Pub. No.: US 2021/0199670 A1
(43) Pub. Date: Jul. 1, 2021

(54) FARBER DISEASE MARKERS AND USES THEREOF

(71) Applicant: Enzyvant Therapeutics GmbH, Basel (CH)

(72) Inventors: Christine Coquery, Durham, NC (US); Brante Sampey, Durham, NC (US)

(73) Assignee: Enzyvant Therapeutics GmbH, Basel (CH)

(21) Appl. No.: 17/195,240

(22) Filed: Mar. 8, 2021

Related U.S. Application Data
(63) Continuation of application No. 16/362,102, filed on Mar. 22, 2019.
Application No. 16/362,102 is a continuation of application No. PCT/US19/23526, filed on Mar. 22, 2019.
(60) Provisional application No. 62/648,775, filed on Mar. 27, 2018.

(51) Int. Cl. G01N 33/68; A61P 3/00

(52) U.S. Cl. CPC G01N 33/6893; A61K 38/00; A61P 3/00; C12Y 305/01023

(57) ABSTRACT

Immune-phenotype markers for Farber disease and their uses are disclosed, as are methods of diagnosing and treating Farber disease based on these markers.

### FIELD OF INVENTION

Methods and biomarkers for determining whether a subject has Farber disease.

### BACKGROUND OF THE INVENTION

Farber disease (acid ceramidase deficiency, lipogranulomatosis) is a rare lysosomal storage disorder caused by mutations in the lysosomal acid ceramidase (ASAH1) gene. Acid ceramidase is responsible for the degradation of ceramide to sphingosine and fatty acid, and a deficiency of acid ceramidase activity leads to the accumulation of ceramide.

Leukocytes play a role in the pathogenesis of Farber disease. Tissue infiltration with foamy histiocytes (lipid-laden macrophages and monocyte-derived populations), in conjunction with inflammation, promotes the formation of granulomas that are characteristic of the disease. Both granulomas and a ceramide-induced chronic inflammatory state likely lead to tissue damage, with connective tissues and joints, lungs, liver, central nervous system and secondary lymphoid organs being the most overtly affected. However, the characterization of the immune cell development and activation leading to Farber disease has not been fully developed.

Treatment of a progressive murine knock-in Asah1^{P361R/P361R} model of Farber disease with recombinant human acid ceramidase, rhAC has been shown to reduce tissue ceramides and inflammation. The disclosures in International Application No. PCT/US18/13509 filed Jan. 12, 2018, and in He et al., “Enzyme replacement therapy for Farber disease: Proof-of-concept studies in cells and mice,” BBA Clin. 2017 Feb. 13; 7:85-96 are incorporated by reference in their entirety.

However, there continues to be a need for improved diagnosis and treatment for Farber disease. The conventional histological evaluation of tissue biopsy to confirm Farber disease is expensive, invasive, and can cause pain, hemorrhage, or even death. A simple test using immune-phenotype markers of Farber disease, e.g., to diagnose Farber's disease, and/or to determine efficacy of treatment of Farber's disease, is highly desirable. The present subject matter fulfills other needs as well as will be discussed herein.

### SUMMARY OF THE INVENTION

In accordance with the description, some embodiments are directed to a method for determining whether a subject has Farber disease, the method comprising detecting the level of at least one marker selected from CD11b⁺Ly6G⁺, SSC^{mid}FSC^{mid}, MHCII⁻CD11b^{hi}, MHCIICD11b⁻Ly6C⁺, MHCII⁻ CD11b^{hi}CD86⁺, CD11b⁺CD38⁺, CD19⁺CD38⁺, CD11b⁺CD206⁺, MHCII+CD11b^{mid}CD23⁺, and CD19⁻CD3⁺ in a biological sample from a subject, wherein if the level of CD11b⁺Ly6G⁺, SSC^{mid}FSC^{mid}, MHCII⁻CD11b^{hi}, MHCII⁺CD11b⁻Ly6C⁺, MHCII⁻ CD11b^{hi}CD86⁺, CD11b⁺CD38⁺, CD19⁺CD38⁺ is higher than a control, the subject has Farber disease; and if the level of CD11b⁺CD206⁺, MHCII⁺CD11b^{mid}CD23⁺, and CD19⁻CD3⁺ is lower than a control, the subject has Farber disease.

In one embodiment, the method further comprises detecting the level of MHCII⁺CD11b⁻Ly6C⁺, in a sample from the subject, wherein a level of MHCII⁺CD11b⁻ Ly6C⁺ that is higher than a control level indicates that the subject has Farber disease.

In one embodiment, the method further comprises detecting the level of MHCII⁻CD11b^{hi} CD86⁺, in a sample from the subject, wherein a level of MHCII⁻CD11b^{hi} CD86⁺ that is higher than a control level indicates that the subject has Farber disease.

In one embodiment, the method further comprises detecting the level of CD11b⁺CD38⁺, in a sample from the subject, wherein a level of CD11b⁺CD38⁺ that is higher than a control level indicates that the subject has Farber disease.

In one embodiment, the method further comprises detecting the level of CD11b⁺CD206⁺, in a sample from the subject, wherein a level of CD11b⁺CD206⁺ that is lower than a control level indicates that the subject has Farber disease.

In one embodiment, the method further comprises detecting the level of CD11b⁺Ly6G⁺, in a sample from the subject, wherein a level of CD11b⁺Ly6G⁺ that is higher than a control level indicates that the subject has Farber disease.

In one embodiment, the method further comprises detecting the level of CD19⁺CD38⁺, in a sample from the subject, wherein a level of CD19⁺CD38⁺ that is higher than a control level indicates that the subject has Farber disease.

In one embodiment, the method further comprises detecting the level of CD19⁻CD3⁺, in a sample from the subject, wherein a level of CD19⁻CD3⁺ that is lower than a control level indicates that the subject has Farber disease.

In some embodiments, the detection is performed by detecting the levels of MHCII⁺CD11b⁻Ly6C⁺ and MHCII⁻CD11b^{hi}CD86⁺ in a sample from the subject, wherein a level of MHCII⁺CD11b⁻Ly6C⁺ and/or MHCII⁻CD11b^{hi}CD86⁺ that is higher than a control level indicates that the subject has Farber disease. In some embodiments, the detection is performed by detecting the level of CD19⁺CD38⁺ in a sample from the subject, and further detecting a level of CD19⁻CD3⁺ in a sample from the subject, wherein a level of CD19⁺CD38⁺ that is higher than a control level and/or a level of CD19⁻CD3⁺ lower than a control level, and the combined detection indicates that the subject has Farber disease. In other embodiments, the detection is performed by detecting the levels of at least four, at least five, at least six, at least seven, at least eight, at least nine, or ten markers, selected from CD11b⁺Ly6G⁺, SSC^{mid}FSC^{mid}, MHCII⁺CD11b^{hi}, MHCII⁺CD11b⁻Ly6C⁺, MHCII⁻CD11b^{hi}CD86⁺, CD11b⁺CD38⁺, CD19⁺CD38⁺, CD11b⁺CD206⁺, MHCII⁺CD11b^{mid}CD23⁺, and CD19⁻CD3⁺ to determine whether a subject has Farber disease.

In some embodiments, the biological sample is a tissue extract sample or a blood sample. In some embodiments, the biological sample is obtained from liver, spleen, lung, or blood.

In some embodiments, the method further comprises administering a therapeutically effective amount of a pharmaceutical composition useful in the treatment of Farber disease. In some embodiments, the composition comprises a recombinant human acid ceramidase (rhAC). In some embodiments, the rhAC is administered in an amount of about 0.1 mg/kg to about 50 mg/kg.

In some embodiments the pharmaceutical composition comprises a human recombinant acid ceramidase in an effective amount of about 1 mg/kg to about 10 mg/kg. In some embodiments, the human recombinant acid ceramidase is RVT-801.

In some embodiments the pharmaceutical composition comprises a human recombinant acid ceramidase in an effective amount of about 1 mg/kg to about 5 mg/kg. In some embodiments, the human recombinant acid ceramidase is RVT-801.

Another embodiment is a kit for performing any of the methods detailed above together with instructions for use in diagnosing Farber disease.

In some embodiments, the kit comprises at least one antibody that specifically binds marker CD11b⁺Ly6G⁺, SSC^{mid}FSC^{mid}, MHCII⁺CD11b^{hi}, MHCII⁺CD11b⁻Ly6C⁺, MHCII⁻CD11b^{hi}CD86⁺, CD11b⁺CD38⁺, CD19⁺CD38⁺, CD11b⁺CD206⁺, MHCII+CD11b^{mid}CD23⁺, or CD19⁻CD3⁺.

Another embodiment is a method for treating Farber disease, the method comprising: detecting a level of at least one marker selected from CD11b⁺Ly6G⁺, SSC^{mid}FSC^{mid}, MHCII⁻CD11b^{hi}, MHCII⁺CD11b⁻Ly6C⁺MHCII⁻CD11b^{hi}CD86⁺, CD11b⁺CD38⁺, CD19⁺CD38⁺, CD11b⁺CD206⁺, MHCII⁺CD11b^{mid}CD23⁺, and CD19⁻CD3⁺ in a sample from a subject, wherein if the level of CD11b⁺Ly6G⁺, SSC^{mid}FSC^{mid}, MHCII⁻CD11b^{hi}, MHCII⁺CD11b⁻Ly6C⁺, MHCII⁻CD11b^{hi}CD86⁺, CD11b⁺CD38⁺, CD19⁺CD38⁺ is higher than a control, the subject has Farber disease; and if the level of CD11b⁺CD206⁺, MHCII⁺CD11b^{mid}CD23⁺, and CD19⁻CD3⁺ is lower than a control, the subject has Farber disease; and administering a therapeutically effective amount of a pharmaceutical composition useful in the treatment of Farber disease.

In some embodiments, the pharmaceutical composition comprises a recombinant human acid ceramidase (rhAC). In some embodiments, the pharmaceutical composition comprises rhAC in an amount of about 0.1 mg/kg to about 50 mg/kg.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 shows a flow cytometry assay that identifies leukocyte subpopulations (black outlines showing distribution of lymphocytes, monocytes, and granulocytes) in spleen of Farber mice stained with live/dead zombie red, as described in Example 1.

FIGS. 2A and 2B show flow cytometry assays for spleen extract from 4 and 8 week old Farber mice and wild-type littermates stained with live/dead zombie red, as described in Example 1 (black outline, live cells).

FIGS. 3A and 3B show flow cytometry assays for lung extract from 4 and 8 week old Farber mice and wild-type littermates stained with live/dead zombie red, as described in Example 1 (black outline, live cells).

FIGS. 4A and 4B show flow cytometry assays for liver extract from 4 and 8 week old Farber mice and wild-type littermates stained with live/dead zombie red, as described in Example 1 (black outline, live cells).

FIGS. 5A and 5B show flow cytometry assays for blood from 4 and 8 week old Farber mice and wild-type littermates stained with live/dead zombie red, as described in Example 1 (black outline, live cells).

FIGS. 6A and 6B show flow cytometry assays for spleen and blood, respectively, from 4 and 8 week old Farber mice and wild-type littermates, as described in Example 1. Cell suspensions were first gated based on expression of CD45 (common leukocyte marker) (black outlines), and further gated for physical parameters, including forward scatter (FSC), a measure of size, and side scatter (SSC), a measure of cell granularity, to select monocytes (SSC^{mid}FSC^{mid}). FIG. 6A shows flow cytometry assays for spleen. FIG. 6B shows flow cytometry assays for blood.

FIGS. 7A and 7B show the frequency of leukocytes (CD45⁺ cells) and monocytes (SSC^{mid}FSC^{mid} cells) of spleen and blood, respectively, from 4 and 8 week old Farber mice and age-matched wild-type littermates obtained from the flow cytometry assays of FIGS. 7A and 7B. FIG. 7A shows the frequency of leukocytes (CD45⁺ cells) and monocytes (SSC^{mid}FSC^{mid} cells) in spleen. FIG. 7B shows the frequency of leukocytes (CD45⁺ cells) and monocytes (SSC^{mid}FSC^{mid} cells) in blood. The arrows indicate the changes of monocyte populations in Farber mice.

FIGS. 8A and 8B show flow cytometry assays for lung and spleen, respectively, from 4 and 8 week old Farber mice and wild-type littermates, as described in Example 1. Cell suspensions were gated based on expression of CD11b (monocyte/granulocyte lineage marker) and MHCII (major histocompatibility complex class II; antigen presentation molecule) to identify subsets of macrophages and dendritic cells (DCs): MHCII⁻CD11b⁻, MHCII⁺CD11b⁻, MHCII⁻CD11b^{mid}, MHCII⁻CD11b^{mid}, and MHCII⁻CD11b^{hi}. FIG. 8A shows flow cytometry assays for lung. FIG. 8B shows flow cytometry assays for spleen.

FIGS. 9A and 9B show quantification of the frequency of subsets of macrophages and dendritic cells (DCs) in lung and spleen, respectively, from 4 and 8 week old Farber mice and wild-type littermates: MHCII⁻CD11b⁻, MHCFII⁺CD11b⁻, MHCII⁺CD11b^{mid}, MHCII⁻CD11b^{mid}, and MHCII⁻CD11b^{hi} from the flow cytometry assays as shown in FIGS. 8A and 8B. FIG. 9A shows quantification of the frequency of subsets of macrophages and dendritic cells (DCs) in lung. FIG. 9B shows quantification of the frequency of subsets of macrophages and dendritic cells (DCs) in spleen.

FIGS. 10A and 10B show flow cytometry assays for liver and blood, respectively, from 4 and 8 week old Farber mice and wild-type littermates, as described in Example 1. Cell suspensions were gated based on expression of CD11b (monocyte/granulocyte lineage marker) and MHCII (major histocompatibility complex class II; antigen presentation molecule) to identify subsets of macrophages and dendritic cells (DCs): MHCII⁻CD11b⁻, MHCII⁺CD11b^{mid}, MHCII⁺CD11b^{mid}, MHCII⁻CD11b^{mid}, and MHCII⁻CD11b^{hi}. FIG. 10A shows flow cytometry assays for liver. FIG. 10B shows flow cytometry assays for blood.

FIG. 11 shows a comparison of MHCII⁻CD11b^{hi} population (activated monocytes) among lung, spleen, liver, and blood from the flow cytometry assays as shown in FIGS. 8A, 8B, 10A, and 10B. The arrows indicate an increase of monocyte populations in lung between Farber mice and wild type.

FIGS. 12A and 12B show MHCII⁺CD11b⁻ population as identified in the flow cytometry assays of FIGS. 8A and 8B, further gated based on expression of Ly6C, to identify subpopulations of MHCII⁺CD11b⁻Ly6C⁺ (pro-inflammatory macrophages and DCs) and MHCII⁺CD11b⁻Ly6C⁻, respectively. FIG. 12A shows MHCII⁺CD11b⁻ population in lung. FIG. 12B shows MHCII⁺CD11b⁻ population in spleen.

FIGS. 13A and 13B show comparison of the MHCII⁺CD11b⁻Ly6C⁺ cells (pro-inflammatory macrophages and DCs) as identified in FIGS. 12A and 12B, from the lung and blood, respectively, of 4 and 8 week old Farber mice and wild-type littermates. FIG. 13A shows comparison of the MHCII⁺CD11b⁻Ly6C⁺ cells in lung. FIG. 13B shows comparison of the MHCII⁺CD11b⁻Ly6C⁺ cells in blood.

FIGS. 14A-B shows distributions and comparisons of expression levels of markers of MHCII⁻CD11b⁺ cells from the flow cytometry assays of FIGS. 8A and 8B. FIG. 14A shows distributions of expression levels of markers of MHCII⁻CD11b⁺ cells from the flow cytometry assays of FIGS. 8A and 8B, based on expression levels for markers, CD23, CD68, and CD86 (activated macrophages), in samples from the lung of 4 and 8 week old Farber mice and wild-type littermates. FIG. 14B shows comparisons of expression levels of markers, as represented by mean fluorescence intensity (MFI), for CD23, CD68, and CD86 (activated macrophages) obtained from the measurements as shown in FIG. 14A.

FIG. 15A-B shows comparison of the total count and frequency of CD11b⁺CD38⁺ cells (pro-inflammatory macrophages and DCs) per 100,000 blood cells of 4 and 8 week old Farber mice and wild-type littermates. FIG. 15A shows comparison of the total count of CD11b⁺CD38⁺ cells (pro-inflammatory macrophages and DCs) per 100,000 blood cells of 4 and 8 week old Farber mice and wild-type littermates. FIG. 15B shows the frequency of CD45b⁺CD206⁺ cells in lung (left panel) and the frequency of CD11b⁺CD206⁺ in blood (right panel), of 4 and 8 week old Farber mice and wild-type littermates.

FIG. 16 shows gating CD45⁺ cells (leukocytes) of Example 1, with CD11b⁺ and/or Ly6G^{+/-} to identify neutrophils (CD11b⁺Ly6G⁺) and non-neutrophils (CD11b⁺Ly6G⁻), from the lung of 4 and 8 week old Farber mice and wild-type littermates.

FIG. 17 shows gating CD45⁺ cells (leukocytes) of Example 1, with CD11b⁺ and/or Ly6G^{+/-} to identify neutrophils (CD11b⁺Ly6G⁺) and non-neutrophils (CD11b⁺Ly6G⁻), from the spleen of 4 and 8 week old Farber mice and wild-type littermates.

FIG. 18 shows gating CD45⁺ cells (leukocytes) of Example 1, with CD11b⁺ and/or Ly6G^{+/-} to identify neutrophils (CD11b⁺Ly6G⁺) and non-neutrophils (CD11b⁺Ly6G⁻), from the liver of 4 and 8 week old Farber mice and wild-type littermates.

FIG. 19 shows gating CD45⁺ cells (leukocytes) of Example 1, with CD11b⁺ and/or Ly6G^{+/-} to identify neutrophils (CD11b⁺Ly6G⁺) and non-neutrophils (CD11b⁺Ly6G⁻), from the blood of 4 and 8 week old Farber mice and wild-type littermates.

FIGS. 20A-20D compare the frequency of neutrophils (CD11b⁺Ly6G⁺) identified as in FIGS. 16-19, in lung, spleen, liver, and blood, respectively, of 4 and 8 week old Farber mice and wild-type littermates according to Example 1. FIG. 20A compares the frequency of neutrophils (CD11b⁺Ly6G⁺) identified as in FIGS. 16-19, in lung. FIG. 20B compares the frequency of neutrophils (CD11b⁺Ly6G⁺) identified as in FIGS. 16-19, in spleen. FIG. 20c compares the frequency of neutrophils (CD11b⁺Ly6G⁺) identified as in FIGS. 16-19, in liver. FIG. 20D compares the frequency of neutrophils (CD11b⁺Ly6G⁺) identified as in FIGS. 16-19, in blood.

FIG. 21 shows CD19⁻ cells (non-B cells) further gated to select T cells as double positive for CD45 and CD3 (black outlines), in spleen of 4 and 8 week old Farber mice and wild-type littermates, as described in Example 1.

FIG. 22 shows CD19⁻ cells (non-B cells) further gated to select T cells as double positive for CD45 and CD3 (black outlines), in lung of 4 and 8 week old Farber mice and wild-type littermates, as described in Example 1.

FIG. 23 shows CD19⁻ cells (non-B cells) further gated to select T cells as double positive for CD45 and CD3 (black outlines), in blood of 4 and 8 week old Farber mice and wild-type littermates, as described in Example 1.

FIG. 24 compares the frequency of T cells (CD19⁻CD3⁺) population identified, as in FIGS. 21-23, in spleen, lung, and blood, respectively, of 4 and 8 week old Farber mice and wild-type littermates. FIG. 24A reports the frequency of T cells (CD19⁻CD3⁺) population in spleen. FIG. 24B reports the frequency of T cells (CD19′ CD3⁺) population in lung. FIG. 24A reports the frequency of T cells (CD19⁻CD3⁺) population in blood.

FIG. 25 shows CD45⁺ cells, further gated based on their expression of CD19 (pan-B cell marker) (black outlines), from spleen of 4 and 8 week old Farber mice and wild-type littermates, as described in Example 1.

FIG. 26 shows CD45⁺CD19⁺ cells (B cells), further gated based on their expression of CD38 (activated lymphocytes, plasmablast marker), from the spleen of 4 and 8 week old Farber mice and wild-type mice according to Example 1.

FIGS. 27A-B compare the frequency of B cells and activated B cells, respectively, in the spleen of 4 and 8 week old Farber mice and wild-type littermates according to Example 1, as obtained from the flow cytometry assays in FIGS. 25 and 26, respectively. FIG. 27A compares the frequency of B cells (CD45⁺CD19⁺) in the spleen of 4 and 8 week old Farber mice and wild-type littermates according to Example 1, as obtained from the flow cytometry assays in FIG. 25. FIG. 27B compares the frequency of activated B cells or plasmablasts (CD19⁺CD38⁺) spleen of 4 and 8 week old Farber mice and wild-type littermates according to Example 1, as obtained from the flow cytometry assays in FIG. 26.

FIG. 28 shows CD45⁺CD19⁺ cells (B cells), further gated based on their expression of CD38 (activated lymphocytes, plasmablast marker), from blood of 4 and 8 week old Farber mice and wild-type littermates according to Example 1.

FIGS. 29A-B compare the frequency of CD45⁺CD19⁺ cells (B cells) and CD19⁺CD38⁺ cells (activated B cells or plasmablasts), respectively in the spleen of 4 and 8 week old Farber mice and wild-type littermates according to Example 1, as obtained from the flow cytometry assays in FIG. 28. FIG. 29A compares the frequency of CD45⁺CD19⁺ cells (B cells) in the spleen of 4 and 8 week old Farber mice and wild-type littermates according to Example 1, as obtained from the flow cytometry assays in FIG. 28. FIG. 29B compares the frequency of CD19⁺CD38⁺ cells (activated B cells or plasmablasts) in spleen of 4 and 8 week old Farber mice and wild-type littermates according to Example 1, as obtained from the flow cytometry assays shown in FIG. 28.

FIGS. 30A and 30B show flow cytometry assays for lung and liver, respectively, of 4 and 8 weeks old Farber mice and wild-type littermates, as described in Example 1, gated for CD45⁺ cells. Black outlines indicate CD45^{hi}SSC^{hi} Population. FIG. 30A shows flow cytometry assays for lung of 4 and 8 weeks old Farber mice and wild-type littermates, as described in Example 1, gated for CD45⁺ cells. Black outlines indicate CD45^{hi}SSC^{hi} Population. FIG. 30B shows flow cytometry assays for liver of 4 and 8 weeks old Farber mice and wild-type littermates, as described in Example 1, gated for CD45⁺ cells. Black outlines indicate CD45^{hi}SSC^{hi} Population.

FIG. 31 shows MHCII⁺CD11b^{mid} cells, further gated with CD23+ (mature B cells, activated macrophages, eosinophils, follicular dendritic cells, and platelets), from spleen of 4 and 8 week old Farber mice and wild-type littermates, as described in Example 1.

FIGS. 32A-B shows MHCII⁺CD11b^{mid} cells further gated with CD23⁺ (mature B cells, activated macrophages, eosinophils, follicular dendritic cells, and platelets), from the lung of 4 and 8 week old Farber mice and wild-type littermates, and their frequency, as described in Example 1. FIG. 32A shows MHCII⁺CD11b^{mid} cells, further gated with CD23⁺ (mature B cells, activated macrophages, eosinophils, follicular dendritic cells, and platelets), from the lung of 4 and 8 week old Farber mice and wild-type littermates, as described in Example 1. FIG. 32B shows comparison of the frequency of MHCII⁺CD11b^{mid}CD23⁺ cells from the flow cytometry assays as shown in FIG. 32A, among the lung of 4 and 8 week old Farber mice and wild-type mice.

FIGS. 33A-D show immune-fingerprints based on all cellular subpopulations identified from the flow cytometry assays, including lung, spleen, liver and blood. FIG. 33A shows an immune-fingerprint based on all cellular subpopulations identified from the flow cytometry assays in the lung of Farber mice according to Example 1. FIG. 33B shows an immune-fingerprint based on all cellular subpopulations identified from the flow cytometry assays in the spleen of Farber mice according to Example 1. FIG. 33C shows an immune-fingerprint based on all cellular subpopulations identified from the flow cytometry assays in the liver of Farber mice according to Example 1. FIG. 33D shows an immune-fingerprint based on all cellular subpopulations identified from the flow cytometry assays in the blood of Farber mice according to Example 1.

FIGS. 34A-E show a representative immunophenotyping gating strategy of mouse splenocytes in a Farber “knock-in” mouse treated with recombinant human acid ceramidase (RVT-801), as described in Example 2. FIG. 34A-E cell populations that were first gated based on size (SSC×FSC) to remove cellular debris from processing (FIG. 34A). This population was further gated based on live and dead cells to remove the cell population that was positive for the Zombie red dye (FIG. 34B). The live cells were then gated to select the CD45+ population (FIG. 34C). This population was further gated to determine the percent of CD45⁺ cells that were Ly6G and CD11b double positive; or neutrophils (FIG. 34D). The remaining population was selected and gated to select for the CD11b⁺MHCII⁻ population to determine the population of activated monocytes per sample type (FIG. 34E).

FIGS. 35A-C show splenic immune cell populations in wild-type (WT) mice, a Farber “knock-in” mouse treated with vehicle (saline), or a Farber mouse treated with repeat doses of recombinant human acid ceramidase (RVT-801). Splenic immune cell populations are elevated in control Farber mice when compared to WT splenic immune cell populations, and are decreased in Farber mice treated with recombinant human acid ceramidase (RVT-801), as described in Example 3. FIG. 35A shows cell populations of CD45⁺CD11b⁺Ly6G⁺ splenic neutrophils. FIG. 35B shows cell populations of CD45⁺CD11b^{hi}MHCII⁻ activated splenic monocytes. FIG. 35C shows an immune-fingerprint based on all cellular subpopulations identified from the flow cytometry assays in 4 week and 8 week Farber mice and 4-8 week WT mice.

FIGS. 36A-C show systemic immune cell populations in WT mice, a Farber “knock-in” mouse treated with vehicle (saline) or a Farber mouse treated with repeat doses of recombinant human acid ceramidase (RVT-801). Systemic immune cell populations are elevated in control Farber mice when compared to WT systemic immune cell populations, and are decreased in Farber mice treated with recombinant human acid ceramidase (RVT-801), as described in Example 4. FIG. 36A shows cell populations of CD45⁺CD11b⁺Ly6C⁺ blood neutrophils. FIG. 36B shows cell populations of CD45⁺CD11b^{hi}MHCII⁻ activated blood monocytes. FIG. 36C is shows an immune-fingerprint based on all cellular subpopulations identified from the flow cytometry assays in 4 week and 8 week Farber mice and 4-8 week WT mice.

FIGS. 37A-D show pulmonary immune cell populations in WT mice, a Farber “knock-in” mouse treated with vehicle (saline), or a Farber mouse treated with repeat doses of recombinant human acid ceramidase (RVT-801), Lung immune cell populations are elevated in control Farber mice when compared to WT lung immune cell populations, and are decreased in Farber mice treated with recombinant human acid ceramidase (RVT-801), as described in Example 5. FIG. 37A shows cell populations of CD45⁺CD11b⁺Ly6G⁺ liver neutrophils. FIG. 37B shows cell populations of CD45⁺CD11b^{hi}MCHCII⁻ activated lung monocytes. FIG. 37C shows cell populations of CD45⁺Ly6C⁺MHCII⁺CD11b⁻ activated lung macrophages. FIG. 37D shows an immune-fingerprint based on all cellular subpopulations identified from the flow cytometry assays in 4 week and 8 week Farber mice and 4-8 week WT mice.

FIGS. 38A-B show hepatic immune cell populations in WT mice, in a Farber “knock-in” mouse treated with saline), or a Farber mouse treated with repeat doses of recombinant human acid ceramidase (RVT-801), Liver immune cell populations are elevated in control Farber mice when compared to WT liver immune cell populations, and are decreased in Farber mice treated with recombinant human acid ceramidase (RVT-801), as described in Example 6. FIG. 38A shows cell populations of CD45⁺CD11b⁺Ly6G⁺ liver neutrophils. FIG. 38B shows cell population of CD45⁺CD11b^{hi}MCHCII⁻ activated liver monocytes.

### DESCRIPTION OF THE SEQUENCES

TABLE 1
provides a listing of certain sequences
referenced herein.

| SEQ | | |
|---|---|---|
| ID | | |
| NO | Description | Sequence |
| 1 | recombinant | MPGRSCVALVLLAAAVSCAVAQHAPPWTEDCRKSTY |
| | human acid | PPSGPTYRGAVPWYTINLDLPPYKRWHELMLDKAPV |
| | ceramidase | LKVIVNSLKNMINTFVPSGKIMQVVDEKLPGLLGNF |
| | (rhAC) | PGPFEEEMKGIAAVTDIPLGEIISFNIFYELFTICT |
| | (amino | SIVAEDKKGHLIHGRNMDFGVFLGWNINNDTWVITE |
| | acid) | QLKPLTVNLDFQRNNKTVFKASSFAGYVGMLTGFKP |
| | | GLFSLTLNERFSINGGYLGILEWILGKKDVMWIGFL |
| | | TRTVLENSTSYEEAKNLLTKTKILAPAYFILGGNQS |
| | | GEGCVITRDRKESLDVYELDAKQGRWYVVQTNYDRW |
| | | KHPFFLDDRRTPAKMCLNRTSQENISFETMYDVLST |
| | | KPVLNKLTVYTTLIDVTKGQFETYLRDCPDPCIGW |
| 2 | rhAC | GGCTCGGTCCGACTATTGCCCGCGGTGGGGGAGGGG |
| | (DNA) | GATGGATCACGCCACGCGCCAAAGGCGATCGCGACT |
| | | CTCCTTCTGCAGGTAGCCTGGAAGGCTCTCTCTCTT |
| | | TCTCTACGCCACCCTTTTCGTGGCACTGAAAAGCCC |
| | | CGTCCTCTCCTCCCAGTCCCGCCTCCTCCGAGCGTT |
| | | CCCCCTACTGCCTGGAATGGTGCGGTCCCAGGTCGC |
| | | GGGTCACGCGGCGGAGGGGGCGTGGCCTGCCCCCGG |
| | | CCCAGCCGGCTCTTCTTTGCCTCTGCTGGAGTCCGG |
| | | GGAGTGGCGTTGGCTGCTAGAGCGATGCCGGGCCGG |
| | | AGTTGCGTCGCCTTAGTCCTCCTGGCTGCCGCCGTC |
| | | AGCTGTGCCGTCGCGCAGCACGCGCCGCCGTGGACA |
| | | GAGGACTGCAGAAAATCAACCTATCCTCCTTCAGGA |
| | | CCAACGTACAGAGGTGCAGTTCCATGGTACACCATA |
| | | AATCTTGACTTACCACCCTACAAAAGATGGCATGAA |
| | | TTGATGCTTGACAAGGCACCAGTGCTAAAGGTTATA |
| | | GTGAATTCTCTGAAGAATATGATAAATACATTCGTG |
| | | CCAAGTGGAAAAATTATGCAGGTGGTGGATGAAAAA |
| | | TTGCCTGGCCTACTTGGCAACTTTCCTGGCCCTTTT |
| | | GAAGAGGAAATGAAGGGTATTGCCGCTGTTACTGAT |
| | | ATACCTTTAGGAGAGATTATTTCATTCAATATTTTT |
| | | TATGAATTATTTACCATTTGTACTTCAATAGTAGCA |
| | | GAAGACAAAAAAGGTCATCTAATACATGGGAGAAAC |
| | | ATGGATTTTGGAGTATTTCTTGGGTGGAACATAAAT |
| | | AATGATACCTGGGTCATAACTGAGCAACTAAAACCT |
| | | TTAACAGTGAATTTGGATTTCCAAAGAAACAACAAA |
| | | ACTGTCTTCAAGGCTTCAAGCTTTGCTGGCTATGTG |
| | | GGCATGTTAACAGGATTCAAACCAGGACTGTTCAGT |
| | | CTTACACTGAATGAACGTTTCAGTATAAATGGTGGT |
| | | TATCTGGGTATTCTAGAATGGATTCTGGGAAAGAAA |
| | | GATGTCATGTGGATAGGGTTCCTCACTAGAACAGTT |
| | | CTGGAAAATAGCACAAGTTATGAAGAAGCCAAGAAT |
| | | TTATTGACCAAGACCAAGATATTGGCCCCAGCCTAC |
| | | TTTATCCTGGGAGGCAACCAGTCTGGGGAAGGTTGT |
| | | GTGATTACACGAGACAGAAAGGAATCATTGGATGTA |
| | | TATGAACTCGATGCTAAGCAGGGTAGATGGTATGTG |
| | | GTACAAACAAATTATGACCGTTGGAAACATCCCTTC |
| | | TTCCTTGATGATCGCAGAACGCCTGCAAAGATGTGT |
| | | CTGAACCGCACCAGCCAAGAGAATATCTCATTTGAA |
| | | ACCATGTATGATGTCCTGTCAACAAAACCTGTCCTC |
| | | AACAAGCTGACCGTATACACAACCTTGATAGATGTT |
| | | ACCAAAGGTCAATTCGAAACTTACCTGCGGGACTGC |
| | | CCTGACCCTTGTATAGGTTGGTGAGCACACGTCTGG |
| | | CCTACAGAATGCGGCCTCTGAGACATGAAGACACCA |
| | | TCTCCATGTGACCGAACACTGCAGCTGTCTGACCTT |
| | | CCAAAGACTAAGACTCGCGGCAGGTTCTCTTTGAGT |
| | | CAATAGCTTGTCTTCGTCCATCTGTTGACAAATGAC |
| | | AGATCTTTTTTTTTTCCCCCTATCAGTTGATTTTTC |
| | | TTATTTACAGATAACTTCTTTAGGGGAAGTAAAACA |
| | | GTCATCTAGAATTCACTGAGTTTTGTTTCACTTTGA |
| | | CATTTGGGGATCTGGTGGGCAGTCGAACCATGGTGA |
| | | ACTCCACCTCCGTGGAATAAATGGAGATTCAGCGTG |
| | | GGTGTTGAATCCAGCACGTCTGTGTGAGTAACGGGA |
| | | CAGTAAACACTCCACATTCTTCAGTTTTTCACTTCT |
| | | ACCTACATATTTGTATGTTTTTCTGTATAACAGCCT |
| | | TTTCCTTCTGGTTCTAACTGCTGTTAAAATTAATAT |
| | | ATCATTATCTTTGCTGTTATTGACAGCGATATAATT |
| | | TTATTACATATGATTAGAGGGATGAGACAGACATTC |
| | | ACCTGTATATTTCTTTTAATGGGCACAAAATGGGCC |
| | | CTTGCCTCTAAATAGCACTTTTTGGGGTTCAAGAAG |
| | | TAATCAGTATGCAAAGCAATCTTTTATACAATAATT |
| | | GAAGTGTTCCCTTTTTCATAATTACTCTACTTCCCA |
| | | GTAACCCTAAGGAAGTTGCTAACTTAAAAAACTGCA |
| | | TCCCACGTTCTGTTAATTTAGTAAATAAACAAGTCA |
| | | AAGACTTGTGGAAAATAGGAAGTGAACCCATATTTT |
| | | AAATTCTCATAAGTAGCATTCATGTAATAAACAGGT |
| | | TTTTAGTTTGTTCTTCAGATTGATAGGGAGTTTTAA |
| | | AGAAATTTTAGTAGTTACTAAAATTATGTTACTGTA |
| | | TTTTTCAGAAATCAAACTGCTTATGAAAAGTACTAA |
| | | TAGAACTTGTTAACCTTTCTAACCTTCACGATTAAC |
| | | TGTGAAATGTACGTCATTTGTGCAAGACCGTTTGTC |
| | | CACTTCATTTTGTATAATCACAGTTGTGTTCCTGAC |
| | | ACTCAATAAACAGTCACTGGAAAGAGTGCCAGTCAG |
| | | CAGTCATGCACGCTGATTGGGTGTGT |
| 3 | rhAC | AAGCTTACCGCCACCATGAACTGCTGCATCGGCCTG |
| | (DNA) | GGTGAGAAGGCGCGTGGCTCGCACCGCGCCAGCTAC |
| | | CCCTCCCTGAGCGCCCTCTTCACCGAGGCGTCCATC |
| | | CTCGGATTCGGGAGCTTCGCCGTCAAGGCACAGTGG |
| | | ACCGAGGATTGCCGCAAGAGTACGTACCCCCCCAGT |
| | | GGCCCGACGTACCGCGGCGCCGTCCCCTGGTACACG |
| | | ATCAACCTGGACCTCCCCCCGTACAAGCGCTGGCAC |
| | | GAGTTGATGCTGGACAAGGCCCCCGTACTGAAGGTC |
| | | ATCGTGAACTCCCTGAAGAACATGATCAACACCTTC |
| | | GTCCCCTCGGGCAAGATCATGCAGGTCGTGGACGAG |
| | | AAGCTGCCCGGGCTCCTCGGCAACTTCCCCGGCCCG |
| | | TTCGAAGAGGAGATGAAGGGCATCGCGGCCGTCACT |
| | | GACATCCCCCTGGGCGAGATCATCAGCTTCAACATC |
| | | TTCTACGAGCTGTTCACCATCTGCACCTCCATCGTA |
| | | GCCGAGGACAAGAAGGGCCACCTGATCCACGGTCGC |
| | | AACATGGACTTCGGCGTCTTCCTGGGCTGGAACATC |
| | | AACAACGACACCTGGGTCATCACCGAGCAGCTGAAG |
| | | CCGCTCACCGTGAACCTCGATTTCCAGCGCAACAAC |
| | | AAGACGGTGTTCAAGGCCAGCTCCTTCGCCGGGTAC |
| | | GTCGGGATGCTCACGGGCTTCAAGCCGGGACTGTTC |
| | | TCGCTGACCCTCAACGAGCGGTTCTCCATCAACGGG |
| | | GGCTACCTCGGCATCCTGGAGTGGATTCTCGGCAAG |
| | | AAGGACGTGATGTGGATCGGCTTCCTCACACGGACC |
| | | GTGCTGGAAAACTCCACTAGTTACGAGGAGGCCAAG |
| | | AACCTGCTGACCAAGACGAAGATCCTGGCCCCGGCA |
| | | TACTTCATCCTGGGCGGCAACCAGTCGGGCGAGGGG |
| | | TGCGTCATCACCCGCGACCGGAAGGAGTCCCTGGAC |
| | | GTCTATGAGCTGGACGCCAAGCAGGGCCGCTGGTAC |
| | | GTCGTCCAGACGAACTACGACCGATGGAAGCACCCC |
| | | TTCTTCCTCGACGACCGGCGCACGCCCGCCAAGATG |
| | | TGCCTGAACCGCACCAGCCAGGAGAACATCTCGTTC |
| | | GAGACGATGTACGACGTGCTGTCGACCAAGCCCGTG |
| | | CTCAACAAGCTGACGGTCTACACCACGCTGATCGAC |
| | | GTGACGAAAGGCCAGTTCGAAACGTACCTGCGGGAC |
| | | TGCCCGGACCCTTGCATCGGCTGGTGATAATCTAGA |
| | | GTCGGGGCGGCCGGCC |
| 4 | rhAC | AAGCTTACCGCCACCATGAACTGCTGCATCGGGCTG |
| | (DNA) | GGAGAGAAAGCTCGCGGGTCCCACCGGGCCTCCTAC |
| | | CCAAGTCTCAGCGCGCTTTTCACCGAGGCCTCAATT |
| | | CTGGGATTTGGCAGCTTTGCTGTGAAAGCCCAATGG |
| | | ACAGAGGACTGCAGAAAATCAACCTATCCTCCTTCA |
| | | GGACCAACGTACAGAGGTGCAGTTCCATGGTACACC |
| | | ATAAATCTTGACTTACCACCCTACAAAAGATGGCAT |
| | | GAATTGATGCTTGACAAGGCACCAGTGCTAAAGGTT |
| | | ATAGTGAATTCTCTGAAGAATATGATAAATACATTC |
| | | GTGCCAAGTGGAAAAATTATGCAGGTGGTGGATGAA |
| | | AAATTGCCTGGCCTACTTGGCAACTTTCCTGGCCCT |
| | | TTTGAAGAGGAAATGAAGGGTATTGCCGCTGTTACT |
| | | GATATACCTTTAGGAGAGATTATTTCATTCAATATT |
| | | TTTTATGAATTATTTACCATTTGTACTTCAATAGTA |
| | | GCAGAAGACAAAAAAGGTCATCTAATACATGGGAGA |
| | | AACATGGATTTTGGAGTATTTCTTGGGTGGAACATA |
| | | AATAATGATACCTGGGTCATAACTGAGCAACTAAAA |
| | | CCTTTAACAGTGAATTTGGATTTCCAAAGAAACAAC |
| | | AAAACTGTCTTCAAGGCTTCCAGCTTTGCTGGCTAT |
| | | GTGGGCATGTTAACAGGATTCAAACCAGGACTGTTC |
| | | AGTCTTACACTGAATGAACGTTTCAGTATAAATGGT |
| | | GGTTATCTGGGTATTCTAGAATGGATTCTGGGAAAG |
| | | AAAGATGTCATGTGGATAGGGTTCCTCACTAGAACA |
| | | GTTCTGGAAAATAGCACAAGTTATGAAGAAGCCAAG |
| | | AATTTATTGACCAAGACCAAGATATTGGCCCCAGCC |
| | | TACTTTATCCTGGGAGGCAACCAGTCTGGGGAAGGT |
| | | TGTGTGATTACACGAGACAGAAAGGAATCATTGGAT |
| | | GTATATGAACTCGATGCTAAGCAGGGTAGATGGTAT |
| | | GTGGTACAAACAAATTATGACCGTTGGAAACATCCC |
| | | TTCTTCCTTGATGATCGCAGAACGCCTGCAAAGATG |
| | | TGTCTGAACCGCACCAGCCAAGAGAATATCTCATTT |
| | | GAAACCATGTATGATGTCCTGTCAACAAAACCTGTC |
| | | CTCAACAAGCTGACCGTATACACAACCTTGATAGAT |
| | | GTTACCAAAGGTCAATTCGAAACTTACCTGCGGGAC |
| | | TGCCCTGACCCTTGTATAGGTTGGTGATAACCTAGG |
| | | GTCGGGGCGGCCGGCC |

In an embodiment, “RVT-801” is a recombinant human acid ceramidase (rhAC) in activated form for the treatment of Farber disease. The alpha and beta subunits of the activated rhAC are joined by a disulfide bond. The molecule is a recombinant human acid ceramidase (rhAC) derived from CHO-M cells transfected with a DNA plasmid vector expressing rhAC. Rvt-801 is based on UniProt KB code: Q13510.

RVT-801 comprises a recombinantly produced acid ceramidase (rhAC) purified to a purity of at least 95% activated form by a process comprising the steps of subjecting the recombinantly produced acid ceramidase to at least two chromatography steps selected from i) cation exchange chromatography; ii) hydrophobic interaction chromatography (HIC); and iii) anion exchange chromatography; and subjecting the recombinantly produced acid ceramidase in solution to one or more viral inactivation steps, wherein the rhAC solution is titrated to a pH of 3.7 or less. The protein sequence of RVT-801 corresponds to SEQ ID NO: 1.

In an embodiment the purification of rhAC may be performed in accordance with the processes disclosed in PCT/2018/052463, filed on Sep. 24, 2018, which is incorporated herein by reference in its entirety. The therapeutic effect of RVT-801 rhAC has been established in a murine model of severe Farber disease (He, et al, 2017) and has been characterized over multiple studies with endpoints describing positive impacts on histopathological and immunological outcomes along with concomitant reduction of accumulated ceramides.

Other embodiments of active ACs and inactive AC precursor proteins that can be used in this and all aspects of the present invention include, without limitation, those set forth in Table 1 of US 2016/0038574, the contents of which are hereby incorporated by reference.

In some embodiments, the rhAC is a protein that is a protein that is a homolog of SEQ ID NO: 1.

In some embodiments, the rhAC is encoded by a nucleic acid molecule of SEQ ID NO: 2.

In some embodiments, the rhAC is encoded by a nucleic acid molecule of SEQ ID NO: 3.

In some embodiments, the rhAC is encoded by a nucleic acid molecule of SEQ ID NO: 4.

In some embodiments, the sequence of rhAC is as defined in GenBank accession number NM_177924.3 or NM_177924.4, each of which is incorporated by reference in its entirety. The nucleotide sequence encoding the protein can be the complete sequence shown in SEQ ID NO: 2, SEQ ID NO: 3, or SEQ ID NO: 4, or be simply the coding region of the sequence The coding region, for example, could be nucleotides 313 to 1500 of SEQ ID NO: 2 or the corresponding coding region found in SEQ ID NO: 3 or SEQ ID NO: 4. However, as is well known to one of skill in the art, the genetic code is degenerate and, therefore other codons can be used to encode the same protein without being outside of what is disclosed. Since the amino acid sequence is known, any nucleotide sequence that encodes the amino acid sequence is acceptable.

In some embodiments, the nucleotide sequence comprises a signal peptide. In some embodiments, the signal peptide is an amino acid sequence encoded by nucleotides 313 to 375 of SEQ ID NO: 2.

In some embodiments, the protein that is produced comprises a signal peptide of amino acid residues 1-21 of SEQ ID NO: 1.

In some embodiments, the protein that is produced does not comprise a signal peptide, such as the signal peptide of amino acid residues 1-21 of SEQ ID NO: 1. In some embodiments, the signal peptide is removed during a post-translational processing where the enzyme is processed into its different subunits. In some embodiments, the nucleotide sequence is codon optimized for the cell that it the protein is being expressed from. In some embodiments, the protein comprises an alpha-subunit, a beta-subunit, and the like.

In some embodiments, the protein that is produced comprises a peptide of amino acid residues 22-142, 45-139, 134-379, 143-395, or 1-395 of SEQ ID NO: 1. The peptide can be a single protein or a polypeptide of different sequences to form the enzyme. In some embodiments, the protein is free of amino acid residues 1-21. These regions can be encoded by a single nucleotide sequence or separate nucleotide sequences or a combination of nucleotide sequences. As discussed herein, any nucleotide sequence encoding the protein can be used and is not limited to the sequence described herein as SEQ ID NO: 2, SEQ ID NO: 3, or SEQ ID NO: 4.

In some embodiments, the rhAC has acid ceramidase (AC) activity but does not have any detectable acid sphingomyelinase activity, such as the rhAC produced in Examples below. The acid sphingomyelinase activity may be removed, for example, by heat inactivation. See, e.g., U.S. Patent Application Publication No. 20160038574, which is incorporated herein in its entirety. Heat inactivation may also remove other contaminating proteins from an rhAC preparation.

In some embodiments, the purified recombinantly produced acid ceramidase has a purity of at least 90%, 93%, 95%, 98%, or 99%, or a purity of 100%.

In some embodiments, the purified recombinantly produced acid ceramidase has no detectable acid sphingomyelinase activity.

In some embodiments, the acid sphingomyelinase activity of the recombinantly produced acid ceramidase is removed without the use of heat.

### DESCRIPTION OF THE EMBODIMENTS

The present application includes markers, methods, devices, reagents, systems, and kits for determining whether a subject has Farber disease. In some embodiments, methods of determining whether a subject has Farber disease using one or more markers are provided. Methods of treating Farber disease in subjects having the described markers are also disclosed.

### A. Definitions

Unless defined otherwise, technical and scientific terms used herein have the meaning commonly understood by one of ordinary skill in the art to which this invention belongs. Although any methods, devices, and materials similar or equivalent to those described herein can be used in the practice of the invention, certain methods, devices, and materials are described herein.

One skilled in the art will recognize many methods and materials similar or equivalent to those described herein may be used in the practice of the present invention. The present invention is in no way limited to the methods and materials described.

All publications, published patent documents, and patent applications cited herein are hereby incorporated by reference to the same extent as though each individual publication, published patent document, or patent application was specifically and individually indicated as being incorporated by reference.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “animal” includes, but is not limited to, humans and non-human vertebrates such as wild, domestic, and farm animals. The animal can also be referred to as a “subject.”

As used herein, “marker” and “marker” are used interchangeably to refer to a target molecule that indicates or is a sign of a normal or abnormal process in an individual or of a disease or other condition in an individual. More specifically, a “marker” or “marker” is an anatomic, physiologic, biochemical, or molecular parameter associated with the presence of a specific physiological state or process, whether normal or abnormal, and, if abnormal, whether chronic or acute. Markers are detectable and measurable by a variety of methods including laboratory assays and medical imaging. In some embodiments, a marker is a target protein.

As used herein, “marker level” and “level” refer to a measurement that is made using any analytical method for detecting the marker in a biological sample and that indicates the presence, absence, absolute amount or concentration, relative amount or concentration, titer, a level, an expression level, a ratio of measured levels, or the like, of, for, or corresponding to the marker in the biological sample. The exact nature of the “level” depends on the specific design and components of the particular analytical method employed to detect the marker.

A “control level” or “control” of a target molecule refers to the level of the target molecule in the same sample type from an individual that does not have the disease or condition, or from an individual that is not suspected of having the disease or condition. A “control level” of a target molecule need not be determined each time the present methods are carried out, and may be a previously determined level that is used as a reference or threshold to determine whether the level in a particular sample is higher or lower than a normal level. In some embodiments, a control level in a method described herein is the level that has been observed in one or more subjects without Farber disease. In some embodiments, a control level in a method described herein is the average or mean level, optionally plus or minus a statistical variation that has been observed in a plurality of normal subjects, or subjects without Farber disease.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Additionally, a term that is used in conjunction with the term “comprising” is also understood to be able to be used in conjunction with the term “consisting of” or “consisting essentially of.”

As used herein, the term “contacting” means bringing together of two elements in an in vitro system or an in vivo system. For example, “contacting” rhAC polypeptide an individual, subject, or cell includes the administration of the polypeptide to an individual or patient, such as a human, as well as, for example, introducing a compound into a sample containing a cellular or purified preparation containing the polypeptide. Additionally, contacting can refer to transfecting or infecting a cell with a nucleic acid molecule encoding the polypeptide.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual. The diagnosis of Farber disease includes distinguishing individuals who have Farber disease from individuals who do not.

An “effective amount” of an enzyme delivered to a subject is an amount sufficient to improve the clinical course of a Farber disease where clinical improvement is measured by any of the variety of defined parameters well known to the skilled artisan.

As used herein, the phrase “integer from X to Y” means any integer that includes the endpoints. For example, the phrase “integer from X to Y” means 1, 2, 3, 4, or 5.

As used herein, the term “isolated” means that the compounds described herein are separated from other components of either (a) a natural source, such as a plant or cell, or (b) a synthetic organic chemical reaction mixture, such as by conventional techniques.

As used herein, the term “mammal” means a rodent (i.e., a mouse, a rat, or a guinea pig), a monkey, a cat, a dog, a cow, a horse, a pig, or a human. In some embodiments, the mammal is a human

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans. As used herein, the phrase “in need thereof” means that the subject has been identified as having a need for the particular method or treatment. In some embodiments, the identification can be by any means of diagnosis. In any of the methods and treatments described herein, the subject can be in need thereof.

As used herein, the term “purified” means that when isolated, the isolate contains at least 90%, at least 95%, at least 98%, or at least 99% of a compound described herein by weight of the isolate.

As used herein, the terms “subject,” “individual” or “patient,” used interchangeably, means any animal, including mammals, such as mice, rats, other rodents, rabbits, dogs, cats, swine, cattle, sheep, horses, or primates, such as humans.

As used herein, the phrase “substantially isolated” means a compound that is at least partially or substantially separated from the environment in which it is formed or detected.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

### B. Marker

In some embodiments, one or more markers are provided for use either alone or in various combinations to determine whether a subject has Farber disease. As described in detail below, exemplary embodiments include the markers provided in Table 2.

TABLE 2
Flow cytometry panel to assess leukocyte-
derived cellular subpopulations

| Surface | | |
|---|---|---|
| marker | Clone | Role |
| CD45 | 104 | Leukocyte common antigen |
| CD11b | M1/70 | Monocyte/granulocyte lineage marker |
| MHCII | M5/114.15.2 | Antigen-presentation (inflammatory cells) |
| Ly6C | HK1.4 | Pro-inflammatory granulocyte marker |
| Ly6G | 1A8 | Neutrophil marker |
| CD86 | GL-1 | Activation of macrophages, dendritic cells, |
| | | T cell, B cell |
| CD206 | C068C2 | Mannose receptor (anti-inflammatory |
| | | macrophages/dendritic cells) |
| CD19 | 500A2 | Pan-B cell marker |
| CD38 | 90 | Activated lymphocytes, plasmablasts |
| CD3 | 6D5 | Pan-T cell marker |
| CD23 | B3B4 | B-cell activation (presentation on mature |
| | | B cells, activated macrophages, eosinophils, |
| | | follicular dendritic cells, and platelets) |

Table 2 lists eleven markers that are useful for distinguishing samples obtained from a subject with Farber disease from samples from a subject that does not have Farber disease.

In some embodiments, one or more markers from Table 2 are provided for use either alone or in various combinations to determine whether a subject has Farber disease or determine the likelihood that the subject has Farber disease. In some embodiments, one or more markers from Table 2 are useful for determining whether a subject has acid ceramidase deficiency, lipogranulomatosis, and/or ceramide-induced chronic inflammatory state.

In some embodiments, one or more of the markers listed in Table 2 are useful to identify subjects at risk of developing Farber disease. In some embodiments, one or more of the markers listed in Table 2 are useful to identify subjects at risk of acid ceramidase deficiency, lipogranulomatosis, and/or ceramide-induced chronic inflammatory state. In some embodiments, one or more markers listed in Table 2 are provided for use either alone or in various combinations to determine whether a subject has Farber disease.

In some embodiments, one or more sets of markers are provided for use either alone or in various combinations to determine whether a subject has Farber disease. As described in detail below, exemplary embodiments include the sets of markers provided in Table 3. The sets of the markers were identified using gating strategy to identify populations expressing specific markers listed in Table 2 on the flow cytometry assays.

TABLE 3

| Set of markers | Cell Type |
|---|---|
| CD11b⁺Ly6G⁺ | Neutrophil |
| SSC^{mid}FSC^{mid} (Size) | Bulk Monocytes |
| MHCII⁻CD11b^{hi} | Activated Monocytes |
| CD11b⁺CD206⁺ | Anti-inflammatory mΦ & DCs |
| MHCII⁺CD11b⁻Ly6C⁺ | Pro-inflammatory mΦ & DCs |
| MHCII⁻CD11b^{mid}CD23⁺ | Activated Pro-inflammatory mΦ & DCs |
| MHCII⁻CD11b^{hi}CD86⁺ | Activated Pro-inflammatory mΦ & DCs |
| CD11b⁺CD38⁺ | Pro-inflammatory mΦ & DCs |
| CD19⁺CD38⁺ | Activated B Cells (PBs) |
| CD19⁻CD3⁺ | Total T Cells |

In some embodiments, a method comprises detecting the level of at least one set of markers listed in Table 3 in a sample from a subject for determining whether a subject has Farber disease.

In some embodiments, a method comprises determining whether a subject has Farber disease, comprising forming a marker panel having N set of markers from the marker sets listed in Table 3, and detecting the level of each set of markers of the panel in a sample from the subject, wherein N is at least one. In some embodiments, N is at least 2, or N is at least 3, or N is at least 4, or N is at least 5, or N is 5, or N is 6, or N is 7, or N is 8, or N is 9, or N is 10. In some embodiments, a method comprises detecting the level of at least five, at least six, at least seven, at least eight, at least nine, or ten sets of markers, selected from CD11b⁺Ly6G⁺, SSC^{mid}FSC^{mid}, MHCII⁻CD11b^{hi}, MHCII CD11b⁻Ly6C⁺, MHCII⁻CD11b^{hi}CD86⁺, CD11b⁺CD38⁺, CD19⁺CD38⁺, CD11b⁺CD206⁺, MHCII⁺CD11b^{mid}CD23⁺, and CD19⁻CD3⁺ to determine whether a subject has Farber disease.

In some embodiments, a method comprises detecting the level of MHCII⁺CD11b⁻Ly6C⁺, in a sample from the subject, wherein a level of MHCII⁺CD11b⁻Ly6C⁺ that is higher than a control level indicates that the subject has Farber disease.

In some embodiments, a method comprises detecting the level of MHCII⁻CD11b^{hi}CD86⁺, in a sample from the subject, wherein a level of MHCII⁻CD11b^{hi}CD86⁺ that is higher than a control level indicates that the subject has Farber disease.

In some embodiments, a method comprises detecting the level of CD11b⁺CD38⁺, in a sample from the subject, wherein a level of CD11b⁺CD38⁺ that is higher than a control level indicates that the subject has Farber disease.

In some embodiments, a method comprises detecting the level of CD11b⁺CD206⁺, in a sample from the subject, wherein a level of CD11b⁺CD206⁺ that is lower than a control level indicates that the subject has Farber disease.

In some embodiments, a method comprises detecting the level of CD11b⁺Ly6G⁺, in a sample from the subject, wherein a level of CD11b⁺Ly6G⁺ that is higher than a control level indicates that the subject has Farber disease.

In some embodiments, a method comprises detecting the level of CD19⁺CD38⁺, in a sample from the subject, wherein a level of CD19⁺CD38⁺ that is higher than a control level indicates that the subject has Farber disease.

In some embodiments, a method comprises detecting the level of CD19⁻CD3⁺, in a sample from the subject, wherein a level of CD19⁻CD3⁺ that is lower than a control level indicates that the subject has Farber disease.

In some embodiments, a method comprises detecting the levels of MHCII⁺CD11b⁻Ly6C⁺and MHCII⁻CD11b^{hi}CD86⁺ in a sample from the subject, wherein a level of MHCFII⁺CD11b⁻Ly6C⁺ and/or MHCII⁻CD11b^{hi}CD86⁺ that is higher than a control level indicates that the subject has Farber disease.

In some embodiments, a method comprises detecting the levels of CD19⁺CD38⁺ and CD19⁻CD3⁺ in a sample from the subject, wherein a level of CD19⁺CD38⁺ is higher than a control level and a level of CD19⁻CD3⁺ is higher than a control level indicates that the subject has Farber disease.

The markers identified herein provide a number of choices for subsets or panels of markers that can be used to effectively identify Farber disease. The markers identified herein provide a number of choices for subsets or panels of markers that can be used to effectively identify acid ceramidase deficiency, lipogranulomatosis, and/or ceramide-induced chronic inflammatory state. Selection of the appropriate number of such markers may depend on the specific combination of markers chosen. In addition, in any of the methods described herein, except where explicitly indicated, a panel of markers may comprise additional markers not shown in Table 2 or 3.

In some embodiments, a method comprises detecting the level of at least one marker listed in Table 2 in a sample from a subject for determining whether a subject has Farber disease.

In some embodiments, a method comprises detecting the level of at least one, at least two, at least three, at least four, at least five, at least six, at least seven, at least eight, at least nine, or at least ten sets of markers listed in Table 3, in a sample from the subject, wherein a level of at least one marker indicates that the subject has Farber disease.

In some embodiments, the markers are present at different levels in individuals with acid ceramidase deficiency compared to individuals without acid ceramidase deficiency. In some embodiments, the markers are present at different levels in individuals with lipogranulomatosis compared to individuals without lipogranulomatosis.

In some embodiments, the markers are present at different levels in individuals with a ceramide-induced chronic inflammatory state compared to individuals without a ceramide-induced chronic inflammatory state. Detection of the differential levels of a marker in an individual can be used, for example, to permit the determination of whether an individual has Farber disease.

In some embodiments, because the methods with a blood sample is non-invasive, any of the markers described herein may be used to monitor individuals for development of Farber disease or monitor individuals at risk of developing Farber disease or acid ceramidase deficiency. By detecting acid ceramidase deficiency at an earlier stage, medical intervention or treatment may be more effective such as treatment with rhAC.

In addition, in some embodiments, a differential expression level of one or more of the markers in an individual over time may be indicative of the individual's response to a particular therapeutic regimen. Thus, one embodiment of the present invention involves a method to determine the efficacy of a Farber disease treatment regimen. In some embodiments, changes in expression of one or more of the markers during follow-up monitoring may indicate that a particular treatment is effective or may suggest that the therapeutic regimen should be adjusted. Levels of expression of one or more markers may be determined prior to beginning a treatment regimen, and/or during a treatment regimen.

In addition to testing marker levels as a stand-alone diagnostic test, marker levels can also be done in conjunction with other Farber disease screening or diagnostic methods. In some instances, methods using the markers described herein may facilitate the medical and economic justification for implementing more aggressive treatments for Farber disease, more frequent follow-up screening, etc. The markers may also be used to begin treatment in individuals at risk of developing Farber disease, but who have not been diagnosed with Farber disease, if the diagnostic test indicates they are likely to develop the disease. In addition to testing marker levels in conjunction with other Farber disease diagnostic methods, information regarding the markers can also be evaluated in conjunction with other types of data, particularly data that indicates an individual's risk for Farber disease.

### C. Detection of Markers

A marker level for the markers described herein can be detected using any of a variety of known analytical methods. In one embodiment, a marker level is detected using a capture reagent. In various embodiments, the capture reagent can be exposed to the marker in solution or can be exposed to the marker while the capture reagent is immobilized on a solid support. In other embodiments, the capture reagent contains a feature that is reactive with a secondary feature on a solid support. The capture reagent is selected based on the type of analysis to be conducted. In some embodiments, capture reagents include but are not limited to antibodies, small molecules, F(ab′)2fragments, single chain antibody fragments, Fv fragments, single chain Fv fragments, ligand-binding receptors, cytokine receptors, and synthetic receptors, and modifications and fragments of these. In some embodiments, capture reagents include antibodies.

In some embodiments, the marker level is detected directly from the marker in a biological sample. In some embodiments, markers are detected using a multiplexed format that allows for the simultaneous detection of two or more markers in a biological sample.

In some such embodiments, the method comprises contacting the sample or a portion of the sample from the subject with at least one capture reagent, wherein each capture reagent specifically binds a marker or a set of markers whose levels are being detected.

Further, in some embodiments, a biological sample may be derived by taking biological samples from a number of individuals and pooling them, or pooling an aliquot of each individual's biological sample. The pooled sample may be treated as described herein for a sample from a single individual, and, for example, if a poor prognosis is established in the pooled sample, then each individual biological sample can be re-tested to determine which individual(s) have Farber disease.

In some embodiments, a fluorescent tag can be used to label a component of the marker/capture reagent complex to enable the detection of the marker level. In various embodiments, the fluorescent label can be conjugated to a capture reagent specific to any of the markers described herein using known techniques, and the fluorescent label can then be used to detect the corresponding marker level.

In some embodiments, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor dye molecule (Thermo Fischer Scientific), such as, for example, AlexaFluor 488, AlexaFluor 532, AlexaFluor 647, AlexaFluor 680, or AlexaFluor 700. In some embodiments, the dye molecule includes a BD Horizon Brilliant™ dye molecule (BD Sciences), such as, for example, BV421, BV510, BV605, BV 650, or BV711. In some embodiments, the dye molecules include Cy5 or Cy7. In some embodiments, the dye molecule includes a first type and a second type of dye molecule, such as, e.g., two different AlexaFluor molecules. In some embodiments, the dye molecule includes a first type and a second type of dye molecule, and the two dye molecules have different emission spectra.

Fluorescence can be measured with a variety of instrumentation compatible with a wide range of assay formats. In some embodiments, the marker levels for the markers described herein can be detected using any analytical methods including, singleplex or multiplexed immunoassays, histological/cytological methods, etc. Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies and fragments thereof are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Flow cytometry methods also may be used for detection of markers. Methods of performing flow cytometry are known in the art. Generally, the cells, preferably blood cells, are incubated with an antibody. In preferred embodiments, the antibody is a monoclonal antibody. It is more preferred that the monoclonal antibody be labeled with a fluorescent marker. If the antibody is not labeled with a fluorescent marker, a second antibody that is immunoreactive with the first antibody and contains a fluorescent marker. After sufficient washing to ensure that excess or non-bound antibodies are removed, the cells are ready for flow cytometry. Flow cytometry offers a short turnaround time between sample preparation, acquisition, and analysis, allows for the accurate enumeration of individual cell subsets (including very rare subsets), and provides an opportunity for detailed molecular phenotyping.

### D. Kits

Any combination of the markers described herein can be detected using a suitable kit, such as for use in performing the methods disclosed herein. Furthermore, any kit can contain one or more detectable labels as described herein, such as a fluorescent moiety, etc. In some embodiments, a kit includes one or more capture reagents (such as, for example, at least one antibody) for detecting one or more markers in a biological sample. In some embodiments, a kit includes optionally one or more software or computer program products for predicting whether the individual from whom the biological sample was obtained has Farber disease. Alternatively, rather than one or more computer program products, one or more instructions for manually performing the above steps by a human can be provided. The kit can also include instructions for using the devices and reagents, handling the sample, and analyzing the data. Further the kit may be used with a computer system or software to analyze and report the result of the analysis of the biological sample. The kits can also contain one or more reagents (e.g., solubilization buffers, detergents, washes, or buffers) for processing a biological sample. Any of the kits described herein can also include, e.g., buffers, blocking agents, antibody capture agents, positive control samples, negative control samples, software and information such as protocols, guidance and reference data.

### E. Methods of Treatment

In some embodiments, following a determination that a subject has Farber disease (or acid ceramide deficiency), the subject undergoes a therapeutic regimen to delay or prevent worsening of the disease. In some embodiments, a subject is given a therapeutic agent, such as rhAC. Exemplary methods of treating Farber disease with rhAC is described in International Application No. PCT/US18/13509 filed Jan. 12, 2018, and in He et al., “Enzyme replacement therapy for Farber disease: Proof-of-concept studies in cells and mice,” BBA Clin. 2017 Feb. 13; 7:85-96 (He et al., 2017), which are incorporated by reference in their entirety.

In some embodiments, methods of monitoring Farber disease are provided. Any method known to the skilled artisan may be used to monitor disease status and the effectiveness the therapy. Clinical monitors of disease status may include but are not limited to ceramide levels, weight, joint length, inflammation, or any other clinical phenotype known to be associated with Farber disease.

In some embodiments, the present methods of determining whether a subject has Farber disease are carried out at a time 0. In some embodiments, the method is carried out again at a time 1, and optionally, a time 2, and optionally, a time 3, etc., to monitor the progression of the disease in the subject. In some embodiments, different markers are used at different time points, depending on the current state of the individual's disease and/or depending on the rate at which the disease is believed or predicted to progress.

As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. For example, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Thus, “treatment of Farber disease” or “treating Farber disease” means an activity that alleviates or ameliorates any of the primary phenomena or secondary symptoms associated with Farber disease or other condition described herein.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

Various pharmaceutical compositions are described herein and can be used based upon the patient's and doctor's preferences. However, in some embodiments, the pharmaceutical composition is a solution. In some embodiments, the pharmaceutical composition comprises cell conditioned media comprising the rhAC. As used herein, the term “cell conditioned media” refers to cell culture media that has been used to culture cells expressing rhAC and where the protein is secreted into the media and then the protein is isolated or purified from the media. In some embodiments, the media is used to treat the subject. The media, for example, can be applied to the skin of a subject to treat any of the conditions, symptoms, or disorders described herein.

In addition to the routes of administration described herein, in some embodiments, the pharmaceutical composition is administered by contacting the skin of the subject. In some embodiments, the administration is parenteral administration. In some embodiments, the administration comprises injecting the pharmaceutical composition to the subject. In some embodiments, the administration is an intraperitoneal injection or intravenous injection.

In some embodiments, methods of treating Farber disease in a subject in need thereof are provided, wherein the method comprises expressing recombinant human acid ceramidase (rhAC) in a cell; isolating the expressed rhAC from the cell; and administering to the subject a pharmaceutical composition comprising the isolated expressed rhAC in an effective amount of about 0.1 mg/kg to about 50 mg/kg.

In some embodiments, the expressing recombinant human acid ceramidase (rhAC) in a cell comprises transferring a vector encoding rhAC into the cell. In some embodiments, the vector comprises a nucleic acid molecule encoding rhAC. In some embodiments, the nucleic acid molecule is a molecule as described herein or any other nucleic acid molecule that encodes the rhAC polypeptide or homolog thereof, which is described in more detail herein. In some embodiments, the vector is a viral vector. For example, the vector can be a retroviral vector or a DNA virus vector, such as adenovirus, AAV, and the like. In some embodiments, the vector is a plasmid. In some embodiments, the vector comprises a promoter operably linked to the rhAC. In some embodiments, the promoter is a constitutive promoter. In some embodiments, the promoter is the SV40 promoter, CMV promoter, EF1 alpha promoter, or any combination thereof, or any other promoter that is active in a mammalian cell.

In some embodiments, the vector is transfected or infected into the cell. The methods of introducing the vector in the cell are not critical and any method can be used to provide sufficient expression of the rhAC polypeptide in the cell.

In some embodiments, the cell is a mammalian cell. In some embodiments, the cell is not a human cell. In some embodiments, the cell is a hamster cell. In some embodiments, the cell is a Chinese hamster ovarian (CHO) cell. In some embodiments, the cell can be grown in a serum-free or substantially free of serum environment. In some embodiments, the cell is derived from a CHO-K1 cell. In some embodiments, the cell is a murine cell. In some embodiments, the cell is a murine myeloma cell. In some embodiments, the cell is a NSO cell. In some embodiments, the effective amount that is administered is as described herein, above and below.

In some embodiments, the pharmaceutical composition is administered as described herein. For example, in some embodiments, the composition is administered to a subject orally, by inhalation, by intranasal instillation, topically, transdermally, parenterally, subcutaneously, intravenous injection, intra-arterial injection, intramuscular injection, intraplurally, intraperitoneally, intrathecally, or by application to a mucous membrane.

As used herein, the term “rhAC” refers to recombinant human acid ceramidase. In some embodiments, the rhAC comprises an amino acid sequence of SEQ ID NO: 1.

In some embodiments, the rhAC is a protein that is a protein that is a homolog of SEQ ID NO: 1. In some embodiments, the rhAC is encoded by a nucleic acid molecule of SEQ ID NO: 2. In some embodiments, the rhAC is encoded by a nucleic acid molecule of SEQ ID NO: 3. In some embodiments, the rhAC is encoded by a nucleic acid molecule of SEQ ID NO: 4. In some embodiments, the sequence is as defined in GenBank accession number NM_177924.3 or NM_177924.4, each of which is incorporated by reference in its entirety. The nucleotide sequence encoding the protein can be the complete sequence shown in SEQ ID NO: 2, SEQ ID NO: 3, or SEQ ID NO: 4, or be simply the coding region of the sequence The coding region, for example, could be nucleotides 313 to 1500 of SEQ ID NO: 2 or the corresponding coding region found in SEQ ID NO: 3 or SEQ ID NO: 4. However, as is well known to one of skill in the art, the genetic code is degenerate and, therefore other codons can be used to encode the same protein without being outside of what is disclosed. Since the amino acid sequence is known any nucleotide sequence that encodes the amino acid sequence is acceptable. In some embodiments, the nucleotide sequence comprises a signal peptide. In some embodiments, the signal peptide is an amino acid sequence encoded by nucleotides 313 to 375 of SEQ ID NO: 2. In some embodiments, the protein that is produced comprises a signal peptide of amino acid residues 1-21 of SEQ ID NO: 1. In some embodiments, the protein that is produced does not comprises a signal peptide, such as the signal peptide of amino acid residues 1-21 of SEQ ID NO: 1. In some embodiments, the signal peptide is removed during a post-translational process where the enzyme is processed into its different subunits. In some embodiments, the nucleotide sequence is codon optimized for the cell that it the protein is being expressed from. In some embodiments, the protein comprises an alpha-subunit, a beta-subunit, and the like. In some embodiments, the protein that is produced comprises a peptide of amino acid residues 22-142, 45-139, 134-379, 143-395, or 1-395 of SEQ ID NO: 1. The peptide can be a single protein or a polypeptide of different sequences to form the enzyme. In some embodiments, the protein is free of amino acid residues 1-21. These regions can be encoded by a single nucleotide sequence or separate nucleotide sequences or a combination of nucleotide sequences. As discussed herein, any nucleotide sequence encoding the protein can be used and is not limited to the sequence described herein as SEQ ID NO: 2, SEQ ID NO: 3, or SEQ ID NO: 4.

In some embodiments, the rhAC has acid ceramidase (AC) activity but does not have any detectable acid sphingomyelinase activity. The acid sphingomyelinase activity may be removed, for example, by heat inactivation. See, e.g., U.S. Patent Application Publication No. 20160038574, which is incorporated herein in its entirety. Heat inactivation may also remove other contaminating proteins from an rhAC preparation.

The term “homolog” refers to protein sequences having between 80% and 100% sequence identity to a reference sequence. Percent identity between two peptide chains can be determined by pair wise alignment using the default settings of the AlignX module of Vector NTI v.9.0.0 (Invitrogen Corp., Carslbad, Calif.). In some embodiments, the homolog has at least, or about, 80, 85, 86, 87, 88, 89, 90, 91, 92, 93, 94, 95, 96, 97, 98, or 99% identity to a sequence described herein, such as SEQ ID NO: 1. In some embodiments, the protein delivered to the subject conservative substitutions as compared to a sequence described herein. Non-limiting exemplary conservative substitutions are shown in Table 4 are encompassed within the scope of the disclosed subject matter. Substitutions may also be made to improve function of the enzyme, for example stability or enzyme activity. Conservative substitutions will produce molecules having functional and chemical characteristics similar to those molecules into which such modifications are made. Exemplary amino acid substitutions are shown in Table 4 below.

TABLE 4
Exemplary Conservative Substitutions:

| Original Residue | Exemplary Conservative Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

As used herein, “inactive acid ceramidase,” “inactive AC,” or “inactive acid ceramidase precursor,” “inactive AC precursor,” or (AC preprotein) refers to AC precursor protein that has not undergone autoproteolytic cleavage into the active form. Inactive AC precursors and active ACs suitable for use in the recombinant acid ceramidase of this and all aspects of the present invention can be homologous (i.e., derived from the same species) or heterologous (i.e., derived from a different species) to the tissue, cells, and/or subject being treated. Acid ceramidase (e.g., AC) precursor proteins undergo autoproteolytic cleavage into the active form (composed of α- and β-subunits). The mechanism of human AC cleavage and activation is reported in (Shtraizent, 2008). This is promoted by the intracellular environment, and, based on highly conserved sequences at the cleavage site of ceramidase precursor proteins across species, is expected to occur in most, if not all, cell types. Thus, ceramidase as used herein includes both active ceramidases and ceramidase precursor proteins, where ceramidase precursor proteins are converted into active ceramidase proteins through autoproteolytic cleavage. Embodiments in which the precursor protein is taken up by the cell of interest and converted into active ceramidase thereby, as well as embodiments in which the precursor protein is converted into active ceramidase by a different cell or agent (present, for example, in a culture medium), are both contemplated.

Active ACs and inactive AC precursor proteins that can be used in this and all aspects of the present invention include, without limitation, those set forth in Table 1 of CIS 2016/0038574, the contents of which are hereby incorporated by reference. in their entirety.

Table 1 of US 2016/0038574 (herein incorporated in its entirety by reference)

TABLE 1
Exemplary Acid Ceramidase Family Members

| | |
|---|---|
| Homo sapiens | |
| UniProt | Q13510, Q9H715, Q96AS2 |
| OMIM | 228000 |
| NCBI Gene | 427 |
| NCBI RefSeq | NP_808592, NP_004306 |
| NCBI RefSeq | NM_177924, NM_004315 |
| NCBI UniGene | 427 |
| NCBI Accession | Q13510, AAC73009, AAC50907 |
| Mus musculus | |
| UniProt | Q9WV54, Q3U8A7, Q78P93 |
| NCBI Gene | 11886 |
| NCBI RefSeq | NP_062708 |
| NCBI RefSeq | NM_019734 |
| NCBI UniGene | 11886 |
| NCBI Accession | AK151208, AK034204 |
| Gallus gallus | |
| UniProt | Q5ZK58 |
| NCBI Gene | 422727 |
| NCBI RefSeq | NP_001006453 |
| NCBI RefSeq | NM_001006453 |
| NCBI UniGene | 422727 |
| NCBI Accession | CAG31885, AJ720226 |
| Pan troglodytes | |
| NCBI Gene | 464022 |
| NCBI RefSeq | XP_519629 |
| NCBI RefSeq | XM_519629 |
| NCBI UniGene | 464022 |
| Caenorhabditis elegans | |
| UniProt | O45686 |
| IntAct | O45686 |
| NCBI Gene | 173120 |
| NCBI RefSeq | NP_493173 |
| NCBI RefSeq | NM_060772 |
| NCBI UniGene | 173120 |
| NCBI Accession | O45686, CAB05556 |
| Danio rerio | |
| UniProt | Q5XJR7 |
| NCBI Gene | 450068 |
| NCBI RefSeq | NP_001006088 |
| NCBI RefSeq | NM_001006088 |
| NCBI UniGene | 450068 |
| NCBI Accession | AAH83231, CB360968 |
| Rattus norvegicus | |
| UniProt | Q6P7S1, Q9EQJ6 |
| NCBI Gene | 84431 |
| NCBI RefSeq | NP_445859 |
| NCBI RefSeq | NM_053407 |
| NCBI UniGene | 84431 |
| NCBI Accession | AAH61540, AF214647 |

Active ACs and inactive AC precursor proteins that can be used in this and all aspects of the present invention include, without limitation, those set forth in Table 1 of Schuchman, E. H. (inventor), Icahn School of Medicine at Mount Sinai (applicant), 2016, Feb. 11, Therapeutic Acid Ceramidase Compositions And Methods Of Making And Using Them, published as U.S. Published Patent Application No. US 2016/0038574 A1.

In an embodiment, recombinant human acid ceramidase (rhAC) in activated form is utilized for the treatment of Farber disease. The alpha and beta subunits of the activated rhAC are joined by a disulfide bond. The molecule is a recombinant human acid ceramidase (rhAC) derived from CHO-M cells transfected with a DNA plasmid vector expressing rhAC. In an embodiment, rhAC is based on UniProtKB Code: Q13510.

In an embodiment, recombinantly produced acid ceramidase (rhAC) is purified to a purity of at least 95% activated form by a process comprising the steps of subjecting the recombinantly produced acid ceramidase to at least two chromatography steps selected from i) cation exchange chromatography; ii) hydrophobic interaction chromatography (HIC); and iii) anion exchange chromatography; and subjecting the recombinantly produced acid ceramidase in solution to one or more viral inactivation steps, wherein the rhAC solution is titrated to a pH of 3.7 or less. In an embodiment, the protein sequence of rhAC corresponds to SEQ ID NO: 1.

In an embodiment, the purification of rhAC may be performed in accordance with the processes disclosed in PCT/2018/052463, filed on Sep. 24, 2018, which is incorporated herein by reference in its entirety. The therapeutic effect of RVT-801rhAC has been established in a murine model of severe Farber disease (He, et al, 2017) and has been characterized over multiple studies with endpoints describing positive impacts on histopathological and immunological outcomes along with concomitant reduction of accumulated ceramides.

In some embodiments, the purified recombinantly produced acid ceramidase has a purity of at least 90%, 93%, 95%, 98%, or 99%, or a purity of 100%.

In some embodiments, the purified recombinantly produced acid ceramidase has no detectable acid sphingomyelinase activity.

In some embodiments, the acid sphingomyelinase activity of the recombinantly produced acid ceramidase is removed without the use of heat.

The term “in combination with” as used herein means that the described agents can be administered to a subject together in a mixture, concurrently as single agents or sequentially as single agents in any order. The term “in combination with” as used herein means that the described agents can be administered to a subject together in a mixture, concurrently as single agents or sequentially as single agents in any order.

As described herein, in some embodiments, the protein is produced from a cell. In some embodiments, the cell is a Chinese Hamster Ovarian cell, “CHO cell.” A nucleic acid sequence encoding the proteins described herein can be genomic DNA or cDNA, or RNA (e.g. mRNA) which encodes at least one of proteins described herein. The use of cDNA requires that gene expression elements appropriate for the host cell be combined with the gene in order to achieve synthesis of the desired protein. The use of cDNA sequences can advantageous over genomic sequences (which contain introns), in that cDNA sequences can be expressed in bacteria or other hosts which lack appropriate RNA splicing systems. One of skill in the art can determine the best system for expressing the protein.

In some embodiments, the protein is produced according to U.S. Patent Application Publication No. 20160038574, which is incorporated by reference in its entirety.

Because the genetic code is degenerate, more than one codon can be used to encode a particular amino acid. Using the genetic code, one or more different oligonucleotides can be identified, each of which would be capable of encoding the amino acid sequences described herein.

The enzyme that is administered to the subject to treat Farber disease or a condition associate therewith can be purified. The term “purified” with referenced to a protein refers to a protein that is substantially free of other material that associates with the molecule in its natural environment. For instance, a purified protein is substantially free of the cellular material or other proteins from the cell or tissue from which it is derived. The term refers to preparations where the isolated protein is sufficiently pure to be analyzed, or at least 70% to 80% (w/w) pure, at least 80%-90% (w/w) pure, 90-95% pure; and, at least 95%, 96%, 97%, 98%, 99%, or 100% (w/w) pure. In some embodiments, the protein is purified from a cell, such as but not limited to a CHO cell.

### Administration, Compositions, and Kits Comprising the Proteins

In some embodiments, the methods comprise administering a therapeutically or prophylactically effective amount of one or more proteins described herein to a subject with Farber disease or suspected of having Farber disease.

Treatment of subjects may comprise the administration of a therapeutically effective amount of the proteins described herein.

The proteins can be provided in a kit as described herein.

The proteins can be used or administered alone or in admixture with an additional therapeutic. Examples of additional therapeutics include, but are not limited to, inhibitors of acid sphingomyelinase (e.g., amitryptiline (Becker et al., “Acid Sphingomyelinase Inhibitors Normalize Pulmonary Ceramide and Inflammation in Cystic Fibrosis,” Am. J. Respir. Cell. Mol. Biol., 42:716-724 (2010), which is hereby incorporated by reference in its entirety) and inhibitors of ceramide synthases (e.g., Shiffmann et al., “Inhibitors of Specific Ceramide Synthases,” Biochimie, 94:558-565 (2012), which is hereby incorporated by reference in its entirety)). The additional therapeutic can also be ceramidase mixtures described in U.S. Patent Application Publication No. 20160038574, which is hereby incorporated by reference in its entirety.

While enzyme replacement therapies (ERTs) can be effective, as shown in our current study for Farber disease where reduction of AC accumulation was demonstrated, antibodies can develop against the drug, i.e., the replacement enzyme that may reduce its efficacy. Here, we have shown that repeat dosages are well tolerated, which supports a treatment regimen of repeated administration of the replacement enzyme resulting in reduction of the symptoms of the disease, particularly the enzyme that is produced according to the methods described herein and, e.g., in U.S. Patent Application Publication No. 20160038574.

In some embodiments, methods of treating Farber disease in a subject in need thereof comprise administering to the subject a pharmaceutical composition comprising a recombinant human acid ceramidase in an effective amount about once a week, once every 2, 3, or 4 weeks, or once a month, for about 10, about 20, or about 30 weeks, 1, 5, 10, or 25 years, or the duration of a patient's life.

Suitable vehicles and their formulation and packaging are described, for example, in Remington: The Science and Practice of Pharmacy (21st ed., Troy, D. ed., Lippincott Williams & Wilkins, Baltimore, Md. (2005) Chapters 40 and 41). Additional pharmaceutical methods may be employed to control the duration of action. Controlled release preparations may be achieved through the use of polymers to complex or absorb the compounds. Another possible method to control the duration of action by controlled release preparations is to incorporate the compounds of into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, interfacial polymerization, for example, hydroxymethylcellulose or gelatin-microcapsules and poly(methylmethacylate)-microcapsules, respectively, or in colloidal drug delivery systems, for example, liposomes, albumin microspheres, microemulsions, nanoparticles, and nanocapsules or in macroemulsions.

Exemplary delivery devices include, without limitation, nebulizers, atomizers, liposomes (including both active and passive drug delivery techniques) (Wang et al., 1997, pH-sensitive immunoliposomes mediate target-cell-specific delivery and controlled expression of a foreign gene in mouse, Proc. Nat'l Acad. Sci. USA 84:7851-5); Bangham et al., 1965, Diffusion of univalent ions across the lamellae of swollen phospholipids, J. Mol. Biol. 13:238-52; Hsu C. C. (inventor), Genentech, Inc. (assignee), 1997, Aug. 5, Method for preparing liposomes, published as U.S. Pat. No. 5,653,996; Lee, K.-D., et al. (inventors), President and Fellows of Harvard College and the University of Pennsylvania, Intracellular delivery of macromolecules, published as U.S. Pat. No. 5,643,599; Holland J. W. (inventor), The University of British Columbia, Bilayer stabilizing components and their use in forming programmable fusogenic liposomes, published as U.S. Pat. No. 5,885,613; Dzau, V. J, and Kaneda, Yasufumi (inventors), Method for producing in vivo delivery of therapeutic agents via liposomes, published as U.S. Pat. No. 5,631,237; and Loughrey, et al. (inventors), The Liposome Company (assignee), Preparation of targeted liposome systems of a defined size distribution, published as U.S. Pat. No. 5,059,421; Wolff et al., 1984, The use of monoclonal anti-Thy1 IgG1 for the targeting of liposomes to AKR-A cells in vitro and in vivo, Biochim. Biophys. Acta 802:259-73), transdermal patches, implants, implantable or injectable protein depot compositions, and syringes. Other delivery systems which are known to those of skill in the art can also be employed to achieve the desired delivery of ceramidase to the desired organ, tissue, or cells

In general, if administering a systemic dose of the protein, it is desirable to provide the recipient with a dosage of protein which is in the range of from about 1 ng/kg-100 ng/kg, 100 ng/kg-500 ng/kg, 500 ng/kg-1 μkg, 1 μkg/kg-100 μkg/kg, 100 μkg/kg-500 μkg/kg, 500 μkg/kg-1 mg/kg, 1 mg/kg-50 mg/kg, 50 mg/kg-100 mg/kg, 100 mg/kg-500 mg/kg (body weight of recipient), although a lower or higher dosage may be administered.

In some embodiments, the effective amount of rhAC that is administered is from about 0.1 mg/kg to about 10 mg/kg. In some embodiments, the effective amount is from about 10 mg/kg to about 50 mg/kg. In some embodiments, the effective amount is from about 10 mg/kg to about 20 mg/kg. In some embodiments, the effective amount is from about 20 mg/kg to about 30 mg/kg. In some embodiments, the effective amount is from about 30 mg/kg to about 40 mg/kg. In some embodiments, the effective amount is from about 40 mg/kg to about 50 mg/kg. In some embodiments, the effective amount is about 1, 2, 3, 4, 5, 6, 7, 8, 9, or 10 mg/kg.

In some embodiments, a subject diagnosed with Farber disease is administered rhAC at about 1 mg/kg to about 5 mg/kg rhAC or about 2 mg/kg to about 5 mg/kg rhAC every two weeks. In one embodiment, the dosage escalates from 1 mg/kg or 2 mg/kg to 5 mg/kg at week 4. If a dose level is not tolerated by an individual subject, the dose for that subject may be reduced from 2 mg/kg to 1 mg/kg, or 5 mg/kg to 2 mg/kg, as appropriate. The rhAC may be administered every 2 weeks for at least 10, 20, or 30 weeks or for the duration of the subject's life. In one exemplary embodiment, a subject is diagnosed with Farber disease and is identified as having: 1) subcutaneous nodules; and/or 2) an acid ceramidase activity value in white blood cells, cultured skin fibroblasts or other biological sources (e.g., plasma) that is less than 30% of control values; and/or 3) nucleotide changes within both alleles of the acid ceramidase gene (ASAH1) that indicate, through bioinformatic, gene expression studies, and/or other methods, a possible loss of function of the acid ceramidase protein. In some embodiments, the subject is administered rhAC every two weeks for 28 weeks. In some embodiments, the delivery of rhAC is by intravenous infusion (e.g., saline infusion). In some embodiments, starting at about 2 mg/kg and escalating to about 5 mg/kg rhAC (e.g., to 5 mg/kg at week 4).

In additional embodiments, method for treating inflammation associated with Farber disease in a subject in need thereof are disclosed, the methods comprising administering to the subject a pharmaceutical composition comprising a recombinant human acid ceramidase (rhAC) in an effective amount of about 1 mg to about 5 mg/kg or about 2 mg/kg to about 5 mg/kg in, for example, once a week, once every two weeks, or once a month repeat dosages for at least 10 or at least 20 weeks, for 28 weeks, or for the duration of subject's life. In some embodiments, the administration is by intravenous infusion. In one embodiment, the method of treating Farber disease in a subject in need thereof comprises administering to the subject a pharmaceutical composition comprising a recombinant human acid ceramidase (rhAC) in an effective amount of about 1 mg to about 5 mg/kg or about 2 mg/kg to about 5 mg/kg in, for example, once a week, once every two weeks, or once a month repeat dosages for at least 10 or 20 weeks, for 28 weeks, or for the duration of subject's life.

The dosage can be administered once a day, twice a day, three times a day, four times a day, once a week, twice a week, once every two weeks, or once a month. In some embodiments, the dose is administered once a week. The treatment may also be given in a single dose schedule, or a multiple dose schedule in which a primary course of treatment may be with 1-10 separate doses, followed by other doses given at subsequent time intervals required to maintain and or reinforce the response, for example, once a week for 1-4 months for a second dose, and if needed, a subsequent dose(s) after several months. Examples of suitable treatment schedules include: (i) 0, 1 month and 6 months, (ii) 0, 7 days and 1 month, (iii) 0 and 1 month, (iv) 0 and 6 months, or other schedules sufficient to elicit the desired responses expected to reduce disease symptoms, or reduce severity of disease. Other treatment schedules, such as, but not limited to, those described above, can also be used.

In certain aspects of the disclosure, the treatment is started when the subject is newborn, under 1, 2, 3, 4, 5, 6, 7, 8, 9, or 10 years of age, or between 1 and 2, 3, 4, 5, 6, 7, 8, 9, 10, 25, 50, 60, 70 or 80 years of age (e.g., between 1 and 2, between 1 and 3, etc.). In some embodiments, the subject is between 16 and 61. In some embodiments, the subject starts treatment at age 16. In some embodiments, the subject is between 12 and 69. In some embodiments, the subject starts treatment at age 12. In some embodiments, the subject is between 19 and 74. In some embodiments, the subject starts treatment at age 19. In some embodiments, the subject is between 4 and 62. In some embodiments, the subject starts treatment at age 4. In some embodiments, the subject is between 7 and 42. In some embodiments, the subject starts treatment at age 7. In some embodiments, the subject is between 1 and 6 months. In some embodiments, the subject starts treatment at newborn. In some embodiments, the subject starts treatment at age 1 month, 2 months, 3 months, 4 months, 5 months, or 6 months. In some embodiments, the subject is between 6 and 43. In some embodiments, the subject starts treatment at age 6. In some embodiments, the subject is between 5 and 31. In some embodiments, the subject starts treatment at age 5. In some embodiments, the subject is between 5 and 57. In some embodiments, the subject is between 5 and 29. In some embodiments, the subject is between 1 and 3. In some embodiments, the subject starts treatment at age 1. In some embodiments, the subject is between 10 and 70. In some embodiments, the subject starts treatment at age 10. In some embodiments, the subject is between 5 and 80, between 10 and 70, between 20 and 75, between 5 and 60, or between 5 and 30 years of age.

In some embodiments, a subject diagnosed with Farber disease is administered rhAC at about 1 mg/kg to about 5 mg/kg rhAC or about 2 mg/kg to about 5 mg/kg rhAC every two weeks. In one embodiment, the dosage escalates from 1 mg/kg or 2 mg/kg to 5 mg/kg at week 4. If a dose level is not tolerated by an individual subject, the dose for that subject may be reduced from 2 mg/kg to 1 mg/kg, or 5 mg/kg to 2 mg/kg, as appropriate. The rhAC may be administered every 2 weeks for at least 10, 20, or 30 weeks or for the duration of the subject's life. In one embodiment, a subject is diagnosed with Farber disease and is identified as having: 1) subcutaneous nodules; and/or 2) an acid ceramidase activity value in white blood cells, cultured skin fibroblasts or other biological sources (e.g., plasma) that is less than 30% of control values; and/or 3) nucleotide changes within both alleles of the acid ceramidase gene (ASAH1) that indicate, through bioinformatic, gene expression studies, and/or other methods, a possible loss of function of the acid ceramidase protein. In some embodiments, the subject is administered rhAC every two weeks for 28 weeks. In some embodiments, the delivery of rhAC is by intravenous infusion (e.g., saline infusion). In some embodiments, starting at about 2 mg/kg and escalating to about 5 mg/kg rhAC (e.g., to 5 mg/kg at week 4). For example, site specific administration may be to body compartment or cavity such as intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, vaginal, rectal, buccal, sublingual, intranasal, or transdermal means.

The therapeutic compositions described herein can be prepared for use for parenteral (subcutaneous, intramuscular or intravenous) or any other administration particularly in the form of liquid solutions or suspensions. The formulation can also be suitable for an injectable formulation. In some embodiments, the injectable formulation is sterile. In some embodiments, the injectable formulation is pyrogen free. In some embodiments, the formulation is free of other antibodies that bind to other antigens other than an antigen described herein.

The therapeutic composition may also include pharmaceutically acceptable adjuvants, excipients, and/or stabilizers, and can be in solid or liquid form, such as tablets, capsules, powders, solutions, suspensions, or emulsions. Such additional pharmaceutically acceptable ingredients have been used in a variety of enzyme replacement therapy compositions and include, without limitation, trisodium citrate, citric acid, human serum albumin, mannitol, sodium phosphate monobasic, sodium phosphate dibasic, polysorbate, sodium chloride, histidine, sucrose, trehalose, glycine, and/or water for injections. In some embodiments, the salts are hydrates (e.g., trisodium citrate dihydrate, citric acid monohydrate, sodium phosphate monobasic monohydrate, and/or sodium phosphate dibasic heptahydrate).

In some embodiments, the pharmaceutical composition is administered as described herein. For example, in some embodiments, the composition is administered to a subject orally, by inhalation, by intranasal instillation, topically, transdermally, parenterally, subcutaneously, intravenous injection, intra-arterial injection, intramuscular injection, intraplurally, intraperitoneally, intrathecally, or by application to a mucous membrane.

The therapeutic compositions described herein can be prepared for use for parenteral (subcutaneous, intramuscular or intravenous) or any other administration particularly in the form of liquid solutions or suspensions. The formulation can also be suitable for an injectable formulation. In some embodiments, the injectable formulation is sterile. In some embodiments, the injectable formulation is pyrogen free. In some embodiments, the formulation is free of other antibodies that bind to other antigens other than an antigen described herein.

A protein of rhAC capable of treating Farber disease or other condition associated with rhAC activity or use to treat a rhAC related pathology, is intended to be provided to subjects in an amount sufficient to affect a reduction, resolution, or amelioration in the related symptom or pathology. Such a pathology, includes the symptoms of Farber disease as described herein in a subject. An amount is said to be sufficient or a “therapeutically effective amount” to “affect” the reduction of symptoms if the dosage, route of administration, and dosing schedule of the agent are sufficient to influence such a response. Responses to the protein can be measured by analysis of subject's affected tissues, organs, or cells as by imaging techniques or by ex vivo analysis of tissue samples. An agent is physiologically significant if its presence results in a detectable change in the physiology of a recipient patient. In some embodiments, an amount is a therapeutically effective amount if it is an amount that can be used to treat, ameliorate or inhibit symptoms of Farber disease that a subject is subject to. Non-limiting examples of such amounts are provided herein, but are not intended to be limited to such amount if context dictates another amount.

The proteins can be formulated according to known methods to prepare pharmaceutically useful compositions, whereby these materials, or their functional derivatives, are combined in admixture with a pharmaceutically acceptable carrier vehicle.

A protein of rhAC capable of treating Farber disease or other condition associated with rhAC activity or use to treat a rhAC related pathology, is intended to be provided to subjects in an amount sufficient to affect a reduction, resolution, or amelioration in the related symptom or pathology. Such a pathology includes the symptoms of Farber disease as described herein in a subject. An amount is said to be sufficient or a “therapeutically effective amount” to “affect” the reduction of symptoms if the dosage, route of administration, and dosing schedule of the agent are sufficient to influence such a response. Responses to the protein can be measured by analysis of subject's affected tissues, organs, or cells as by imaging techniques or by ex vivo analysis of tissue samples. An agent is physiologically significant if its presence results in a detectable change in the physiology of a recipient patient. In some embodiments, an amount is a therapeutically effective amount if it is an amount that can be used to treat, ameliorate, or inhibit symptoms of Farber disease that a subject is subject to. Non-limiting examples of such amounts are provided herein, but are not intended to be limited to such amount if context dictates another amount.

In some embodiments, efficacy of treatment is assessed by any of the following means:
Percent change from baseline in net nodule (≥5 mm) count after treatment with rhAC for 28 weeks;
Percent change from baseline in net nodule (≥10 mm) count and comparison to placebo after treatment with rhAC for 28 weeks;
Percent change from baseline in total nodule count (regardless of size) and comparison to placebo after treatment with rhAC for 28 weeks;
Change and percent change from baseline of joint range of motion in selected joints and comparison to placebo after treatment with rhAC for 28 weeks;
Change and percent change from baseline of 6-minute walk distance and comparison to placebo after treatment with rhAC for 28 weeks;
Change and percent change from baseline of pulmonary function tests and comparison to placebo after treatment with rhAC for 28 weeks;
Change and percent change from baseline of FDT score and comparison to placebo after treatment with rhAC for 28 weeks;
Change and percent change from baseline in Z-score of body weight and height for age during treatment with rhAC or placebo over 28 weeks.

In some embodiments, pharmacokinetics of RVT-801 following administration to Farber mice or healthy mice at different doses is assessed based on noncompartmental methods. Noncompartmental pharmacokinetics methods estimate the exposure to a drug by estimating the area under the curve of a concentration-time graph, among others, with the follow metrics know in the art:

TABLE 5

| Characteristics | Description |
|---|---|
| Dose (D) | Amount of drug administered |
| Area Under the Curve | The integral of the concentration-time curve: |
| (AUC) | AUC_{0-τ} = ∫ₜ^{t+τ} C dt |
| Elimination half-life | The time required for the concentration of the |
| (t_{1/2}) | drug to reach half of its original value: |
| | ${t}_{1 / 2} = \frac{ln ( 2 )}{{k}_{e}}$ |
| Cₘₐₓ | Maximum observed concentration |
| Tₘₐₓ | Time of maximum observed concentration |
| Tₗₐₛₜ | Time of final quantifiable concentration |
| AUCₗₐₛₜ | Area under the concentration-time curve from |
| | 0 to last quantifiable time point |
| Vz/F | Apparent volume of distribution following |
| | extravascular administration |
| CL/F | Apparent clearance following extravascular |
| | administration |

In some embodiments, tissue-specific efficacy of treatment is assessed by determining tissue-specific pharmacokinetics of RVT-801 based on the above described noncompartmental pharmacokinetics methods.

In some embodiments, Human Equivalent Dose (HED) of RVT-801 corresponding to effective dose for Farber disease mice is estimated. Nonclinical assessments of HED herein have been based on two methods: 1) FDA guidance for scaling between nonclinical species and humans by body surface area (BSA), and 2) organ:bodyweight ratios between species for liver and spleen as the major tissues for ceramide accumulation and in which uptake of RVT-801 predominated.

Scaling by body surface area: The HED, benchmarking against the Farber mouse MED (maximally effective dose) and based on BSA and body weight for a human adult or child (where HED=animal dose*(animal bodyweight/human bodyweight)0.33 indicates a dose of ˜0.81 mg/kg for a 60 kg adult and ˜1.2 mg/kg for a 15 kg child.

Scaling by organ: body weight ratios: PK studies demonstrated the mechanism for clearance from the vasculature is associated with uptake and/or distribution into tissues, thus the tissue weight-to-bodyweight ratio may impact dose and the BSA model may not be sufficiently predictive on its own. Using HED=MED*(Tissuehuman/BWhuman)/(Tissuemouse/BWmouse) a 10 mg/kg dose in mouse is equivalent to a ˜3-5 mg/kg dose in an adult human or a ˜4-5 mg/kg dose in a 15 kg child based on liver and spleen in human adults and children.

In some embodiments, HED may be determined by combining the two scaling approaches above.

Kits, which are described herein and below, are also provided which are useful for carrying out embodiments described herein. In some embodiments, the kits comprise a first container containing or packaged in association with the above-described polypeptides. The kit may also comprise another container containing or packaged in association solutions necessary or convenient for carrying out the embodiments. The containers can be made of glass, plastic or foil and can be a vial, bottle, pouch, tube, bag, etc. The kit may also contain written information, such as procedures for carrying out the embodiments or analytical information, such as the amount of reagent contained in the first container means. The container may be in another container apparatus, e.g. a box or a bag, along with the written information.

Yet another aspect provided for herein is a kit for treating Farber disease. In some embodiments, the kit comprises at least one container comprising a rhAC polypeptide or a nucleic acid molecule encoding the same. In some embodiments, the kit comprises a container comprising a cell that is configured to express rhAC. In some embodiments, the cell is a CHO cell. In some embodiments, the kit comprises conditioned media from a cell that expresses rhAC. In some embodiments, the conditioned media is from a CHO cell.

The subject matter is now described with reference to the following examples. These examples are provided for the purpose of illustration only and the claims should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### EXAMPLES

### Example 1

Materials and Methods

Animal Selection and Sample Collection

Farber mice (Asah1P361R/P361R) and approximately age-matched wild-type CD-1 mice (parental strain of Asah1P361R/P361R) were maintained, according to He et al., 2017, which is incorporated by reference in its entirety.

Whole blood, livers, lungs and spleens were collected from 4 to 8 week old age-matched Farber and wild-type mice (n=3) for characterization by flow cytometry.

Blood samples were collected from all animals per group by cardiac puncture or other approved means to generate the maximum volume blood sample from each mouse. The blood samples were collected into individual Lithium Heparin vials and gently inverted several times to disperse the anticoagulant. Blood samples were separated into two equal aliquots. One aliquot was frozen for subsequent lipid profiling. The second aliquot was placed into an appropriately labeled polystyrene tube and placed on ice until processing for flow cytometry.

Tissues were collected at necropsy immediately following collection of the terminal blood sample. Complete, intact livers, spleen, and lung were collected from up to three (3) animals per group. Each tissue was removed and gently blotted dry. Each tissue was placed into an individual, pre-labeled vial which has been massed (with cap) prior to tissue collection. The mass of the capped vial including the tissue was measured. No buffers, preservatives, or antibiotics was added to the tissues. Tissue masses (i.e. sample+vial mass−vial mass) was reported. Prior to freezing the tissue, a portion of the liver, spleen and lung (approximately 0.025 g) designated for analysis via flow cytometry were removed and placed into an appropriately sized polystyrene petri dish containing 3-5 mL Phosphate Buffered Saline (PBS, pH 7.4; Gibco). Petri dishes containing samples were stored on ice until processing to a single-cell suspension for exploratory endpoints. The mass used for flow cytometry was recorded. Immediately following mass determination of each piece, all tissue samples intended for lipid analyses were frozen for storage at −70° C. ahead of shipment to the bioanalytical lab.

Tissue Processing to Single Cell Suspension

(a) Blood

Equal volume of PBS was added to each blood sample, and 2 mL of 1×FACS Lysing Solution (BD Bioscience) was further added. The sample was gently vortexed and incubated for 10 minutes in the dark at room temperature, and washed by centrifuge three times. Cells were resuspended at 20×106 cells/mL in Assay Buffer (5% Normal Rat Serum (NRS) in PBS). Samples were stored on ice until staining.

(b) Spleen & Liver

Using the frosted portion of two microscope slides, the spleen or liver tissue was homogenized. The slides were with the PBS provided in the petri dish to ensure all cells are collected in the PBS. Following homogenization, all PBS (now containing single cells, as well as small pieces of tissue) were harvested and filter homogenated through a Falcon tube with integrated strainer. After centrifuge at 300×g for 5 minute and supernatant removal, cells were re-suspended in 100 μL PBS followed by adding 2 mL of 1×FACS Lysing Solution. Then, the cell suspension was vortexed gently and incubated for 10 minutes in the dark at room temperature. After washing by centrifuge three times, cells were re-suspended at 20×106 cells/mL in Assay Buffer (5% NRS in PBS). Samples were stored on ice until staining.

(c) Lung

Digestion media was prepared with RPMI 1640 Medium (GlutaMAX™ Supplement, Thermo Fischer Scientific), 1 mg/mL Collagenase II (Fisher Scientific), and 5 U/mL DNAseI (Sigma). Lung was cut into small pieces and suspended in 5 ml digestion media per lung in a petri dish, and was incubated with shaking at 37° C. for approximately 1.5 hours (but not longer than 2 hours). Using the frosted portion of two microscope slides, the lung tissue was homogenized. The slides were with the digestion media provided in the petri dish to ensure all cells are collected. Following homogenization, all digestion media (now containing single cells, as well as small pieces of tissue) were harvested and filter homogenated through a Falcon tube with integrated strainer. After centrifuge at 300×g for 5 minute and supernatant removal, cells were re-suspended in 100 μL PBS followed by adding 2 mL of IX FACS Lysing Solution. Then, the cell suspension was vortexed gently and incubated for 10 minutes in the dark at room temperature. After washing by centrifuge three times, cells were re-suspended at 20×106 cells/mL in Assay Buffer (5% NRS in PBS). Samples were stored on ice until staining.

Flow Cytometry Sample Staining

Staining procedure was performed as follows: 1:200 live/dead Zombie red was added to cells after primary staining accordingly to the manufacturer's instructions. Following staining of surface markers and identification of live/dead populations, cells were re-suspended in 300 uL of BD FACS/Lysing Fixative to lyse any red blood cells. Controls were used from the antibody optimization study. A set of antibodies for staining the samples were prepared as follows.

TABLE 6
Flow ctyometry panel to assess
monocyte-derived cellular subpopulations

| Marker | Clone | Fluorophore | Company | Catalog # |
|---|---|---|---|---|
| CCR2 | SA203G11 | BV421 | BioLegend | 150605 |
| CD68 | FA-11 | A1exa647 | | 137004 |
| CD11c | N418 | BV711 | | 117349 |
| MHCII | M5/114.15.2 | BV510 | | 107635 |
| CD86 | GL-1 | BV605 | | 105037 |
| CD206 | C068C2 | A1exa488 | | 141710 |
| CD11b | M1/70 | BV650 | | 101259 |
| CD45 | 104 | PE-Cy7 | | 109830 |
| Ly6G | 1A8 | APC-Cy7 | | 127624 |
| Ly6C | HK1.4 | PE-CF594 | | 128044 |
| CX3CR1 | SA011F11 | PE | | 149006 |
| F4/80 | BM8 | PE-Cy5 | | 123112 |
| CD23 | B3B4 | A1exa700 | | 101632 |
| live/dead | NA | Fixable Red | Thermo | L34972 |
| marker | | | Fisher | |

TABLE 7
Flow ctyometry panels to assess
lymphocyte cellular subpopulations

| Marker | Clone | Fluorophore | Company | Catalog # |
|---|---|---|---|---|
| CD3 | 500A2 | A1exa700 | BioLegend | 152316 |
| CD19 | 6D5 | A1exa488 | | 115521 |
| CD4 | GK1.5 | APC-Cy7 | | 100414 |
| CD8 | 43-6.7 | BV650 | | 100742 |
| CD62L | MEL-14 | BV605 | | 104438 |
| CD44 | IM7 | PE | | 103008 |
| CD45 | 104 | PE-Cy7 | | 109830 |
| CCR7 | 4B12 | PE-Cy5 | | 120114 |
| CD127 | A7R34 | BV711 | | 135035 |
| MHCII | M5/114.15.2 | BV510 | | 107635 |
| CD38 | 90 | Alexa647 | | 102716 |
| CCR6 | BV421 | 29-2L17 | | 129818 |
| CXCR3 | CXCR3-173 | PE-CF594 | | 126534 |
| live/dead | NA | Fixable Red | Thermo | L34972 |
| marker | | | Fisher | |

Volume of antibodies used was determined during a titration experiment. 100 μL of cell samples were added to a 96-well V-bottom plate. A set of single stain controls (100 μL/well) was prepared for compensation and a set of fluorescence minus one (FMO) controls was prepared for each tissue using pooled samples from all animals. Plates were spun down at 500×g for 5 minutes. Buffer was removed and samples were re-suspended with 100 μL of 1 μg/mL TruStain FcX Block (BioLegend, 101320)) for approximately 10 minutes on ice. Following this incubation, 100 μL of appropriate antibody set was added to each sample, and the sample plates were covered and incubated for approximately 30 minutes on ice. Then, the sample plates were spun down at 500×g for 5 minutes to remove supernatant. Cells were re-suspended in 200 μL of assay buffer (5% NRS in PBS). The sample plates were spun down at 500×g for 5 minutes to remove supernatant, and cells were re-suspended in 200 μL of Fixative Buffer (BD Bioscience, 339860). Samples were stored covered at 4° C.

### Flow Cytometry Sample Acquisition and Analysis

Samples were acquired on a BD Bioscience LSRII instrument using Diva software. Single stained samples were used for appropriate adjustment of voltages to ensure optimal signal to noise, application of compensation matrix, and appropriate labeling of all fluorophore channels. One million events or the maximum volume of the sample were recorded for each sample. Flow cytometric data obtained were analyzed using FlowJo v10 software.

### Results

Flow cytometric data were analyzed to compare the cellular composition and activation status in blood, and liver, spleen, and lung tissues, collected from 4 to 8 weeks old Farber mice (Asah1^{P361R/P361R}) and age-matched wild-type mice.

### Evaluation of Viability Across Tissues

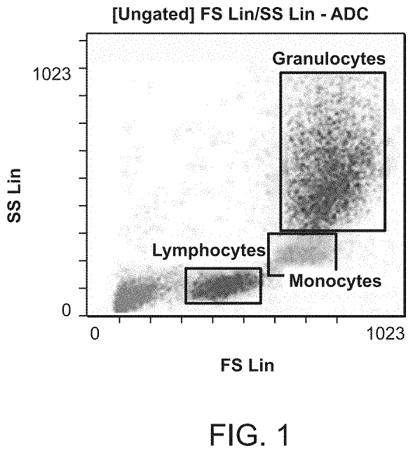

The flow cytometry assays as shown in FIGS. 1-5B show information about bulk cell distribution and general state of cell health in wild type mice. Note that monocytes are identified as SSC^{mid}FSC^{mid}. Any alterations from this distribution may be indicative of disease state.

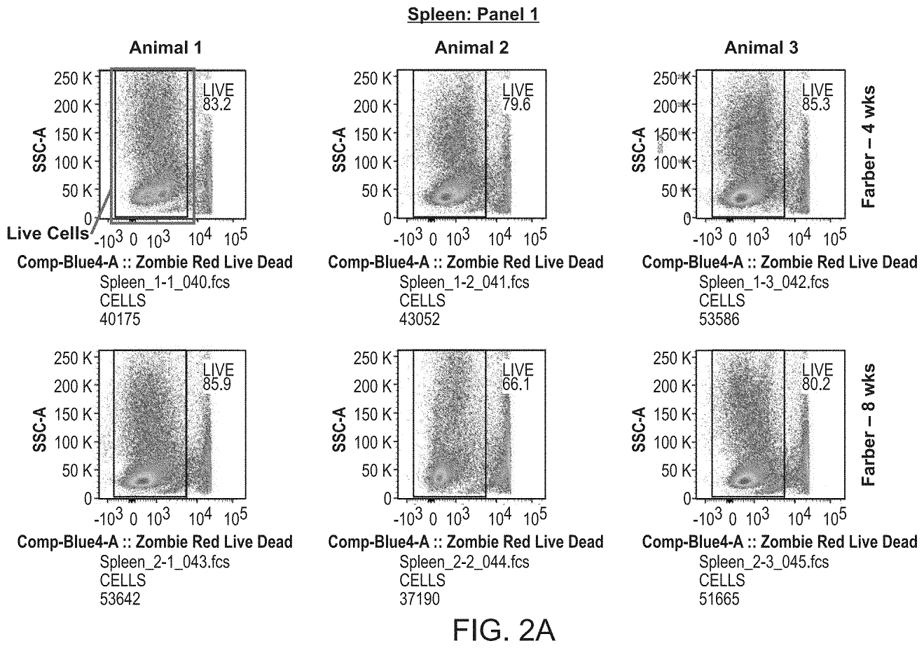

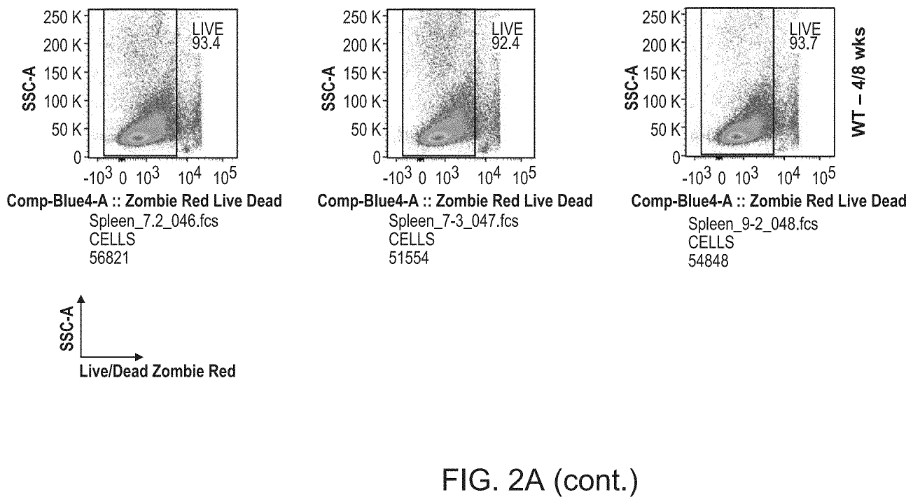

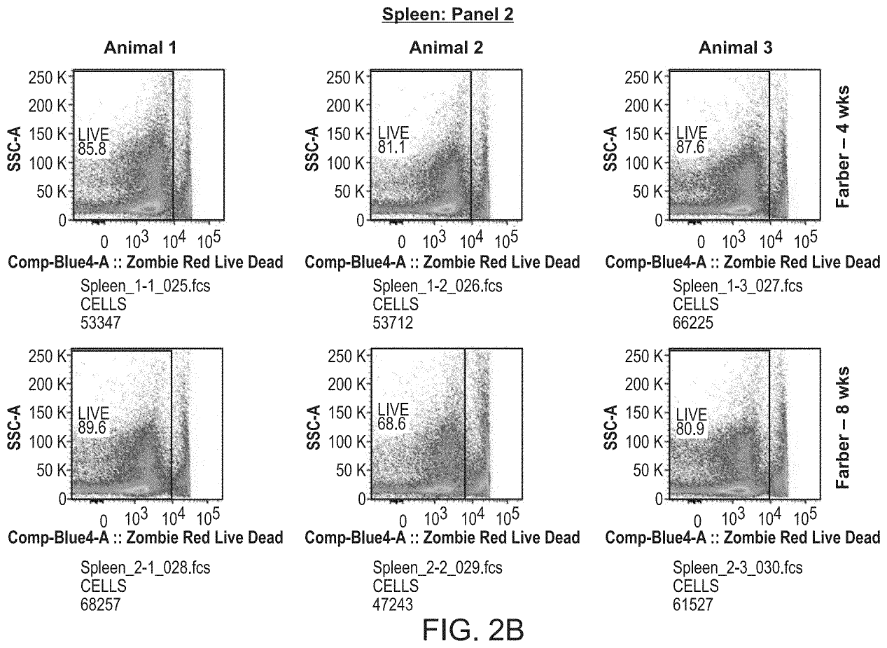

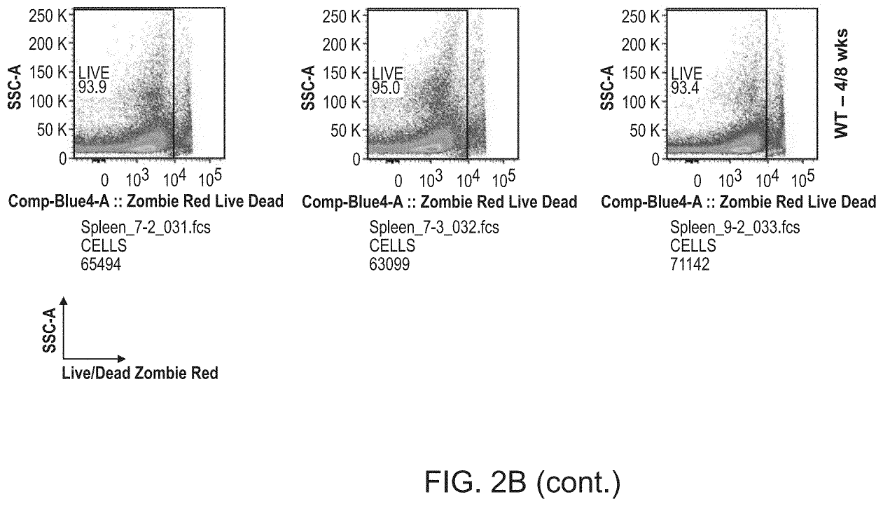

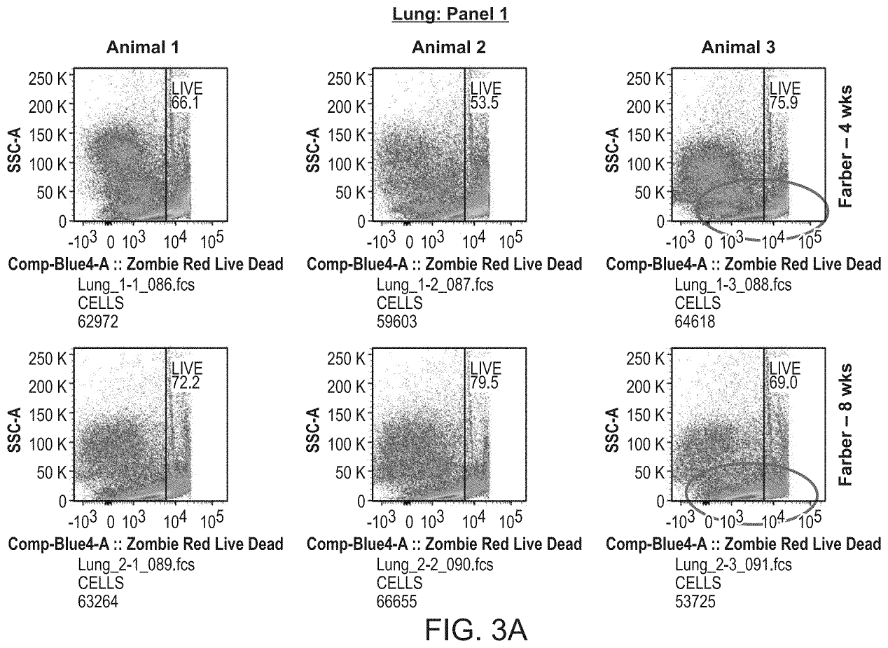

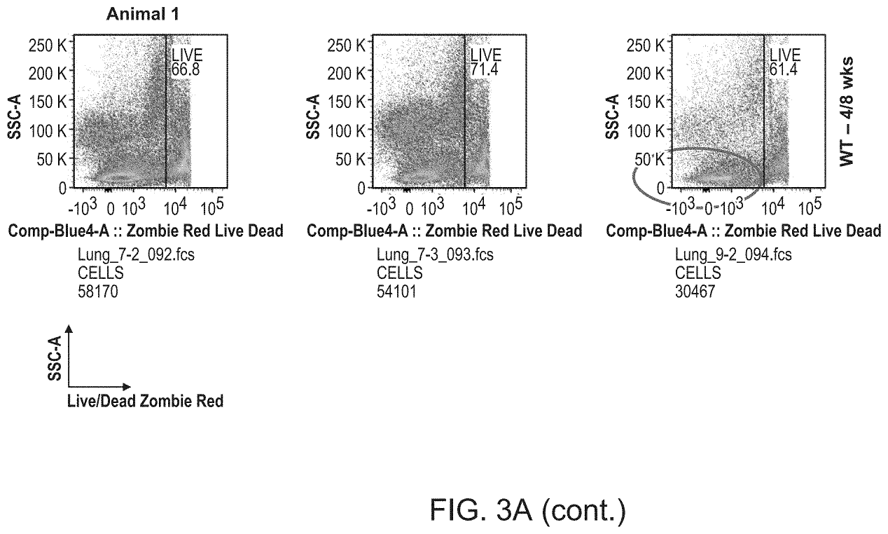

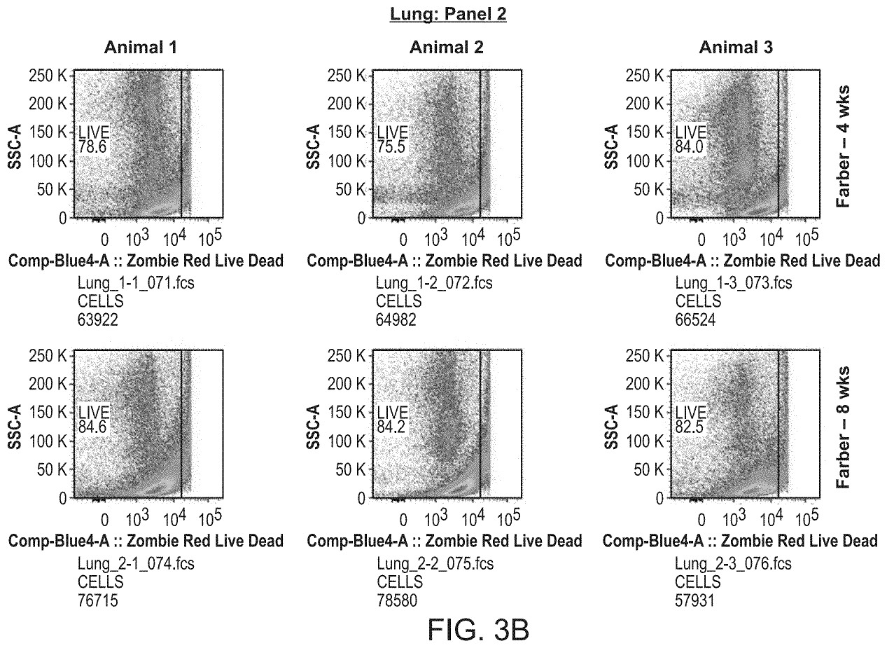

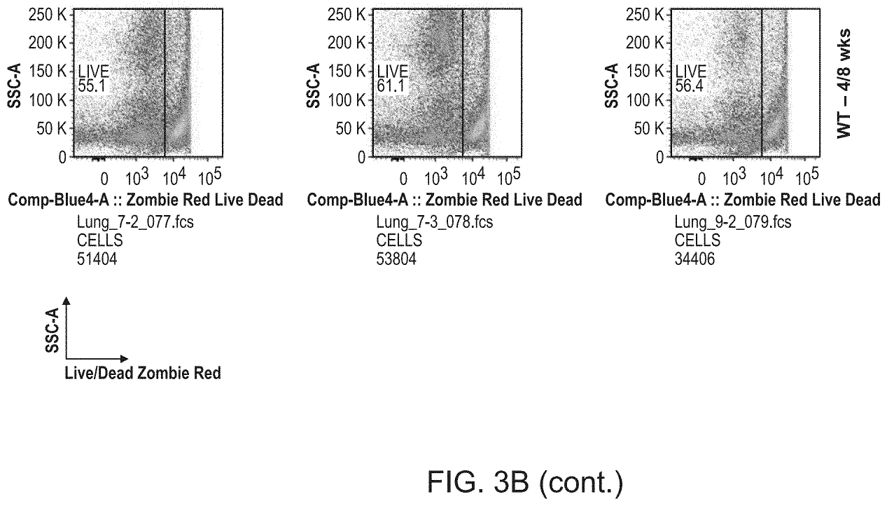

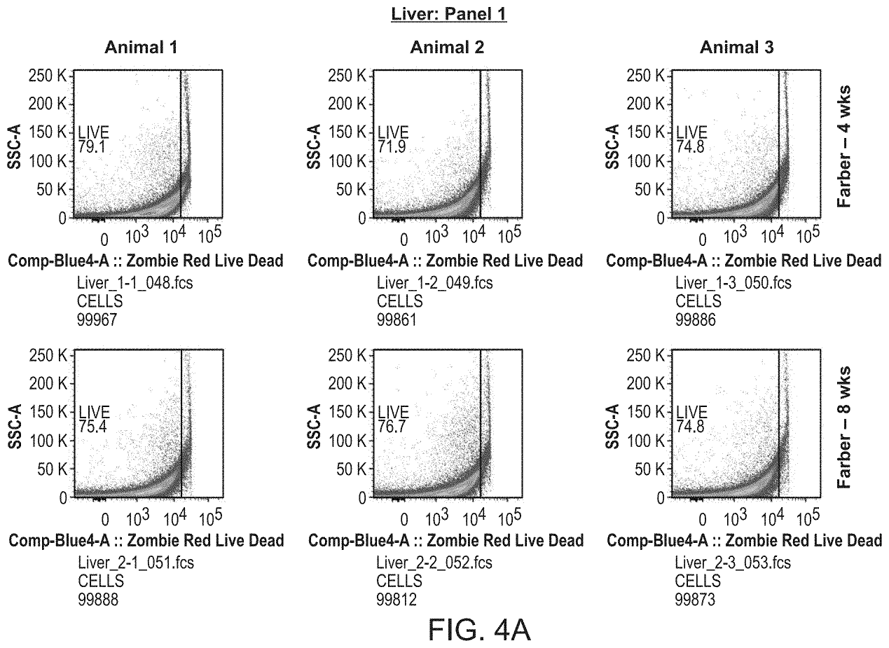

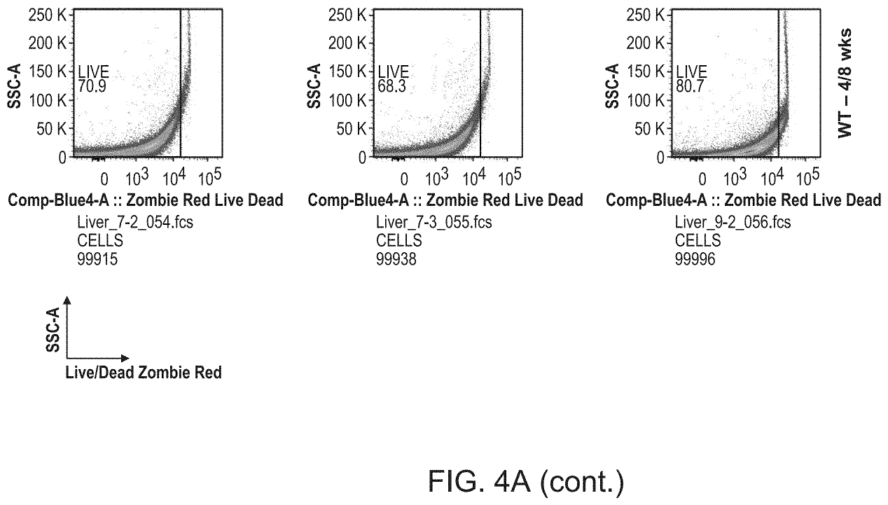

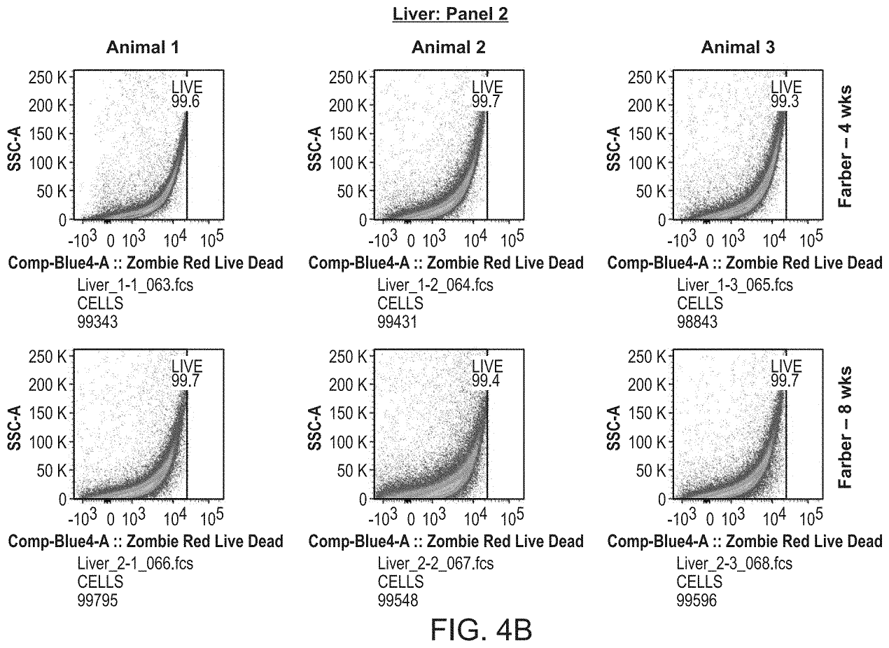

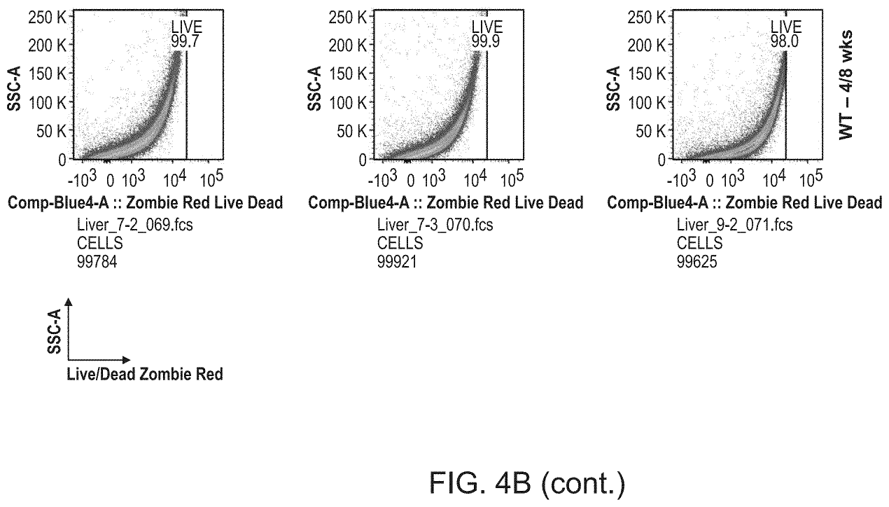

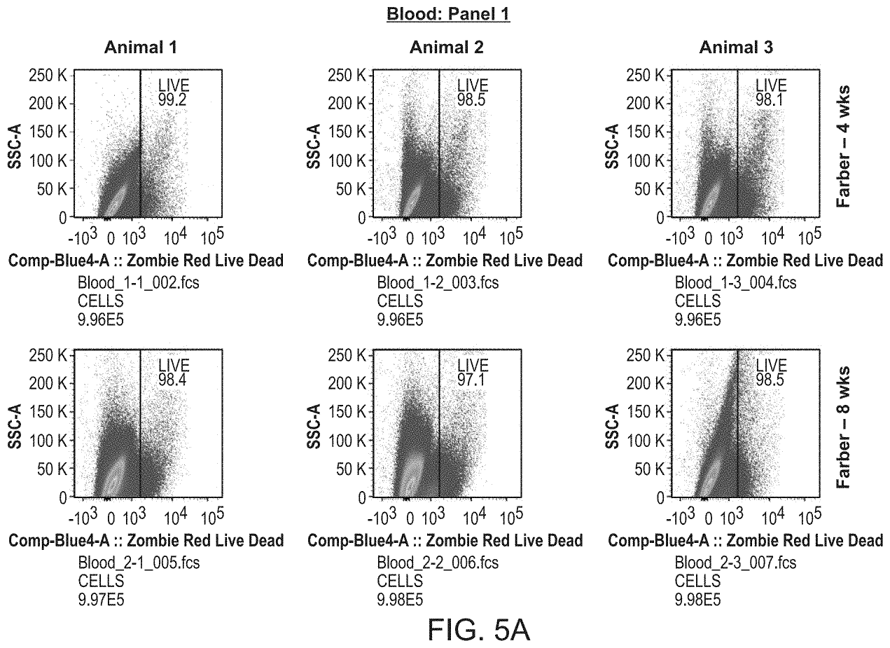

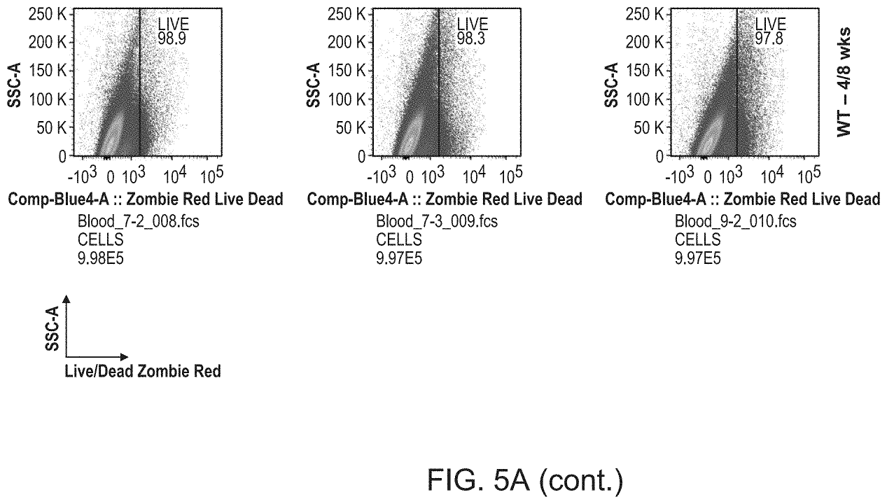

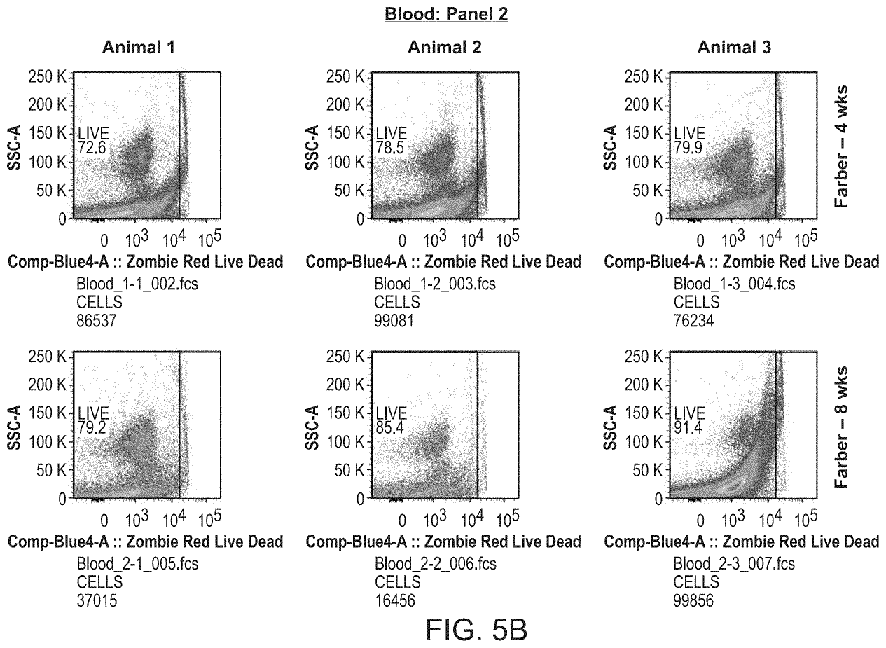

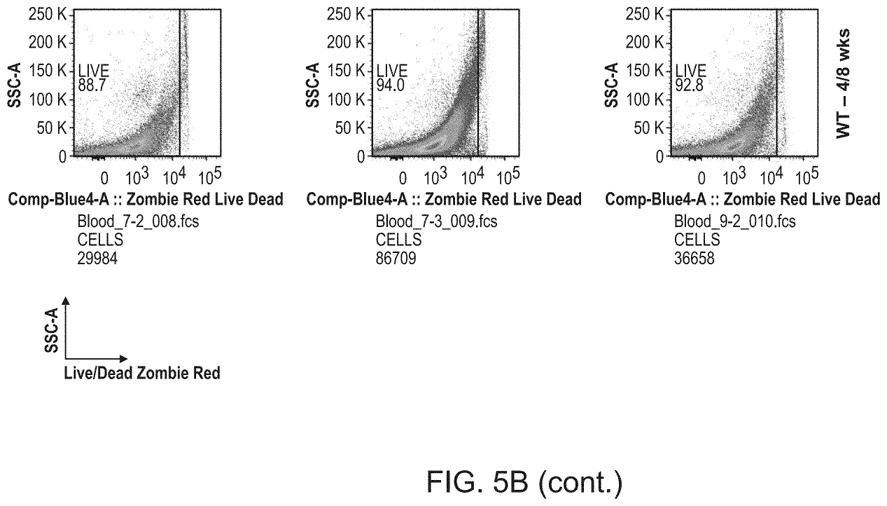

Spleen showed notable differences in cell viability between Farber mice and wild type mice. Reduced spleen viability in Farber mice compared to wild type controls may be related to direct effect of ceramide or via inflammatory processes (FIGS. 2A and 2B). Lung viability in Farber Mice was similar to wild type controls (FIGS. 3A and 3B), which may be reflective of ability to distinguish only a single stage of cell death. Similar liver and blood viability in Farber mice compared to wild type controls were observed (FIGS. 4A, 4B, 5A, and 5B).

CD45⁺

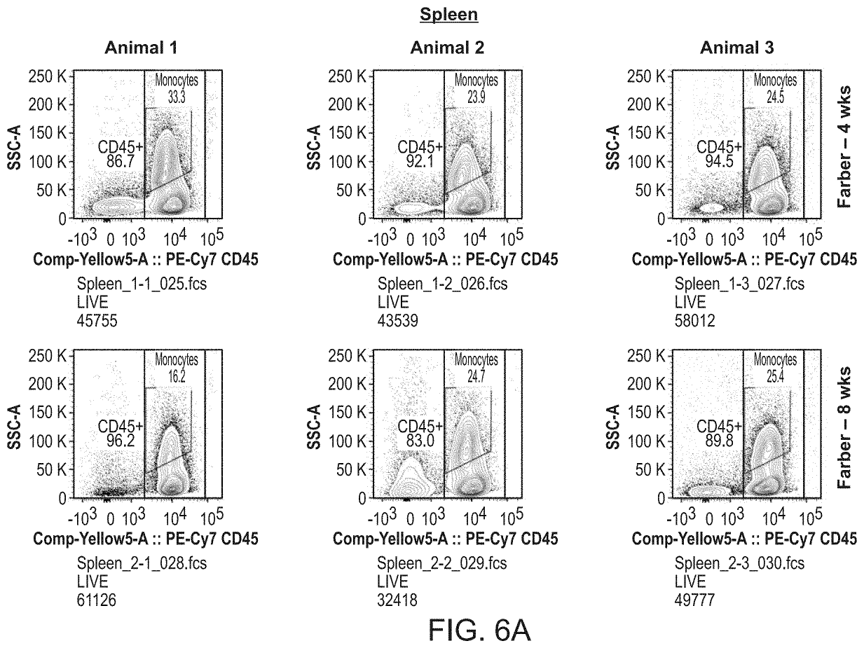

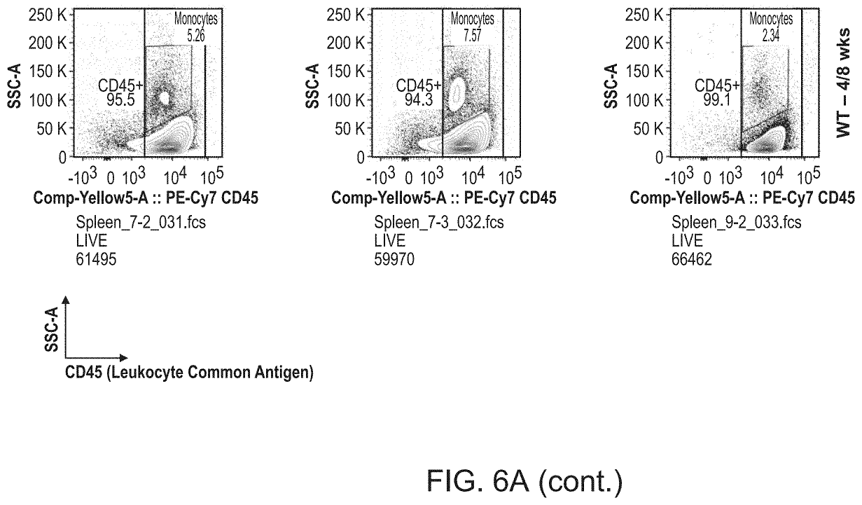

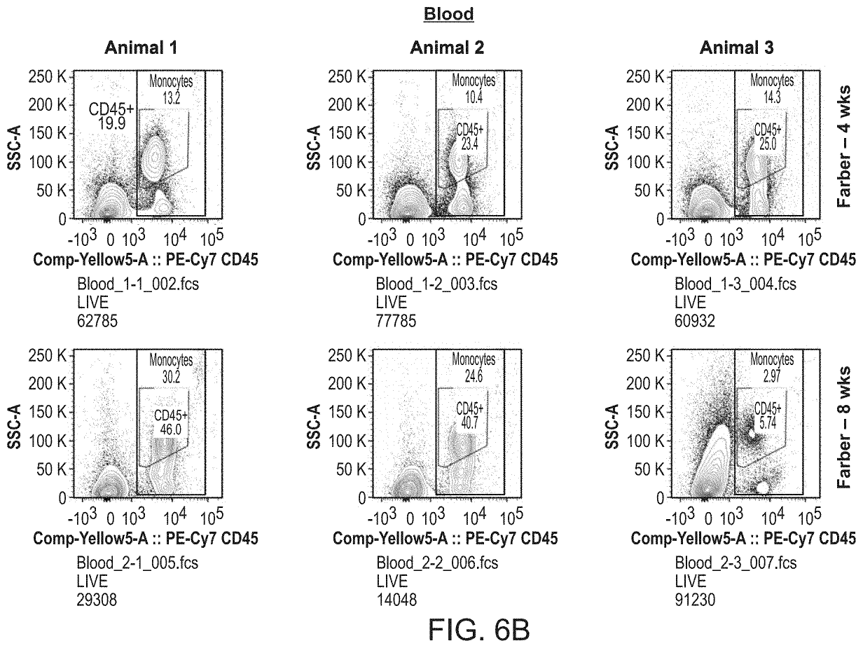

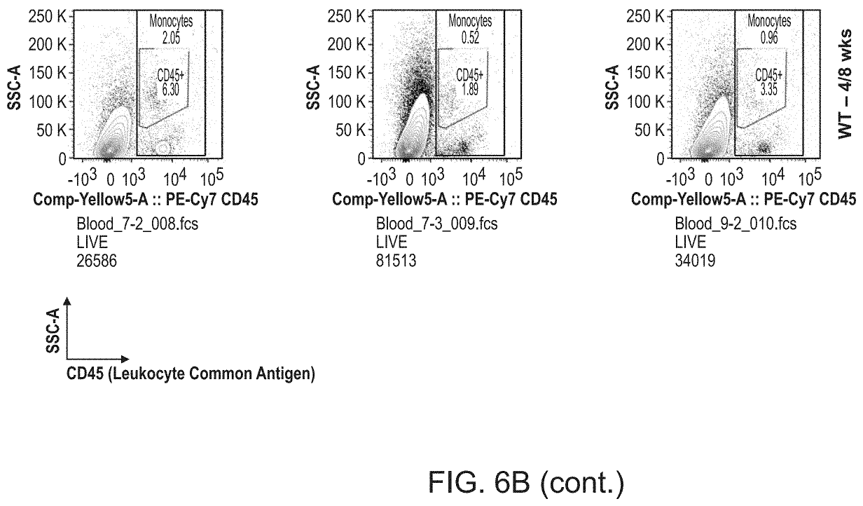

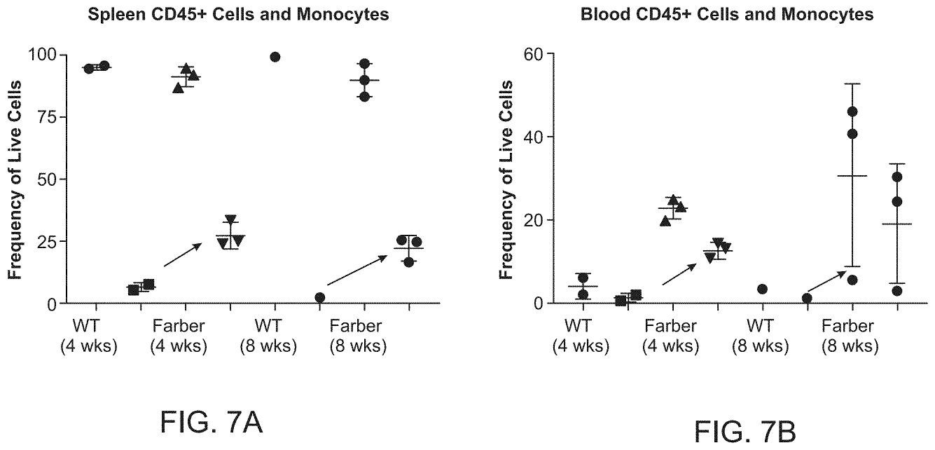

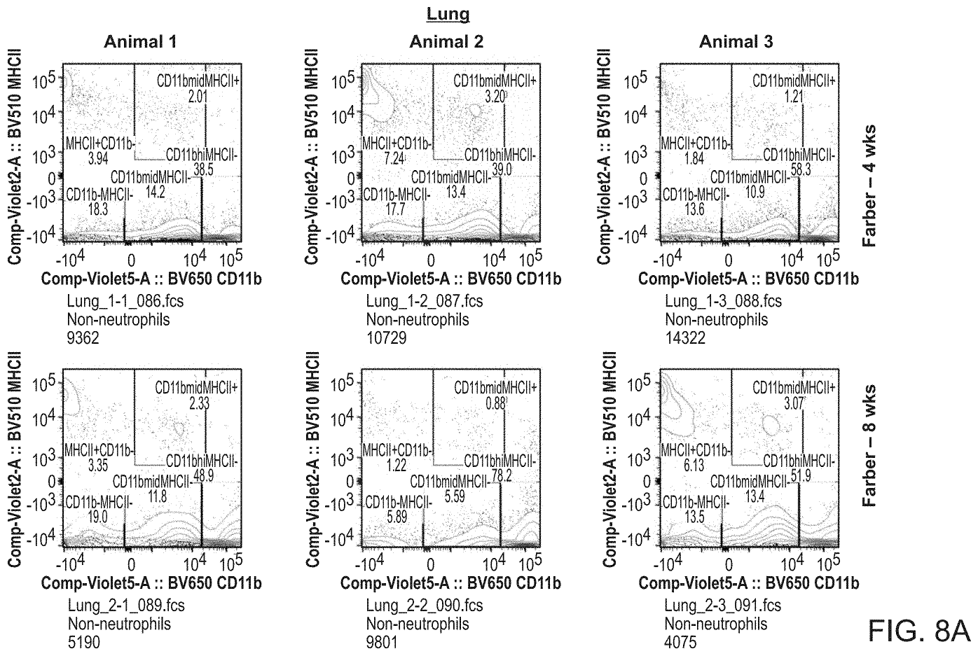

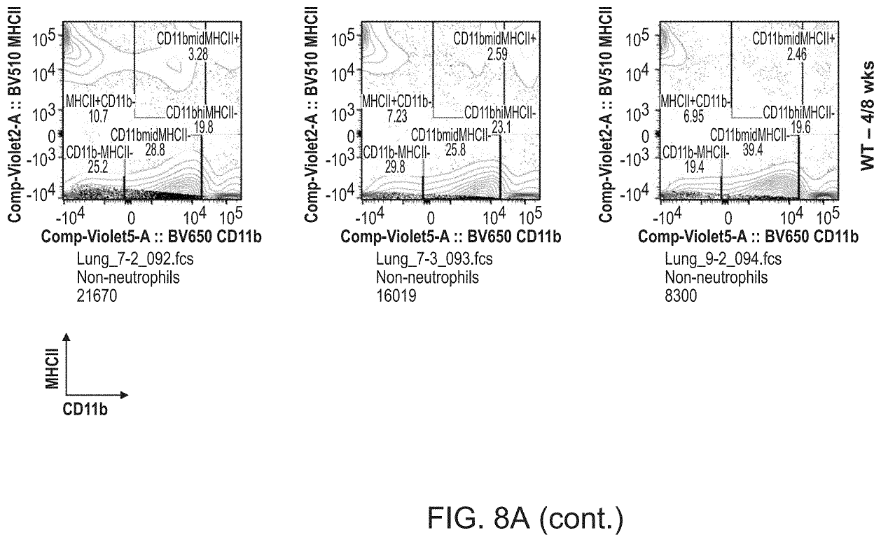

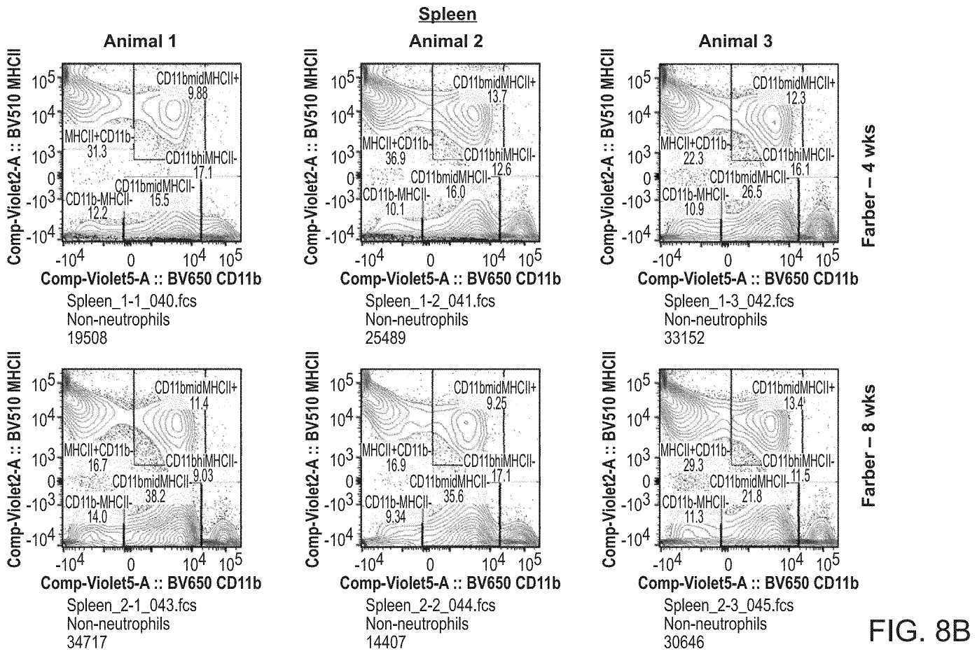

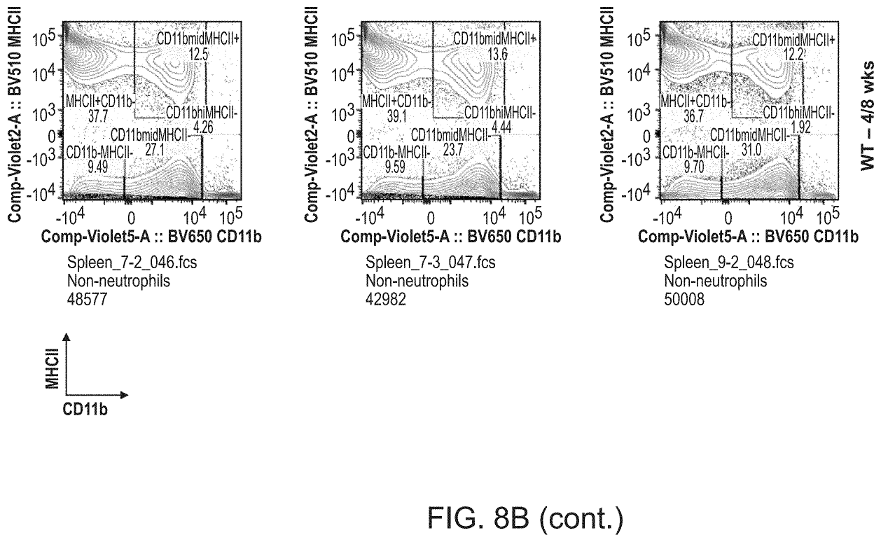

Increased frequency of leukocytes (CD45⁺) cells or changes in cellular distribution is mediated in large part by chronic inflammation or infection. Notable changes were made with regards to the % of CD45⁺ cells and the makeup of the CD45⁺ compartment in Farber mice and wild type mice. FIGS. 6A, 6B, 7A, and 7B compare the population of leukocytes (CD45⁺ cells) and monocytes (SSC^{mid}/FSC^{mid}) in spleen and blood between 4 and 8 week old Farber mice and age-matched wild-type mice. The frequency of leukocytes (CD45⁺ cells) remained comparably similar levels in spleen. As shown in FIGS. 7A and 7B (indicated by arrows), the frequency of monocytes increased in Farber mice as compared to the wild type mice. Moreover, the frequency of monocytes increased more than 5-fold in the spleen of Farber mice as compared to the wild type. This correlates with the peripheral frequency reflecting the changes in the spleen tissue.

MHCII⁻CD11b^{hi} (Activated Monocytes)

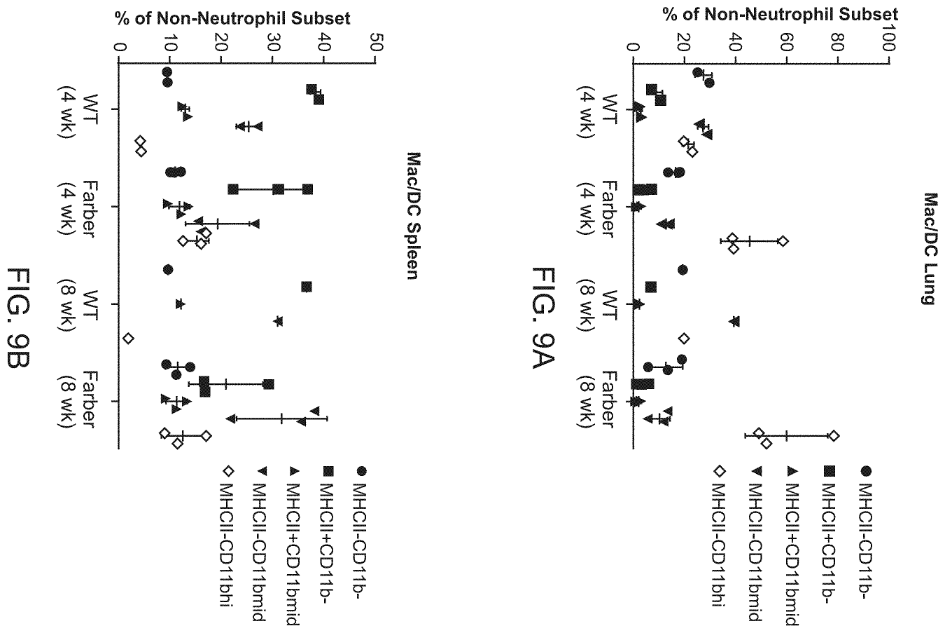

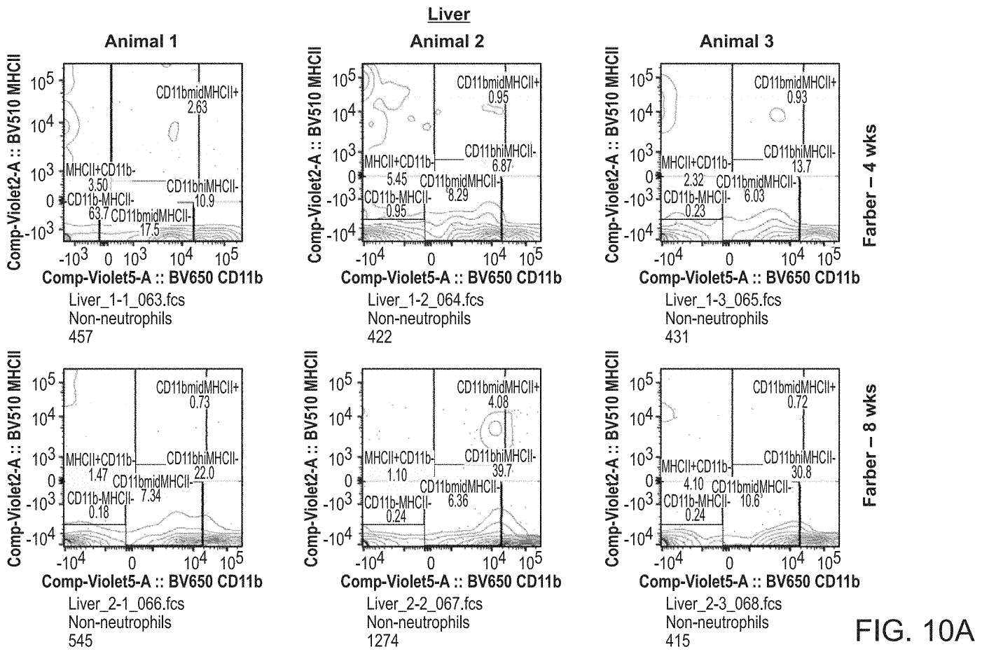

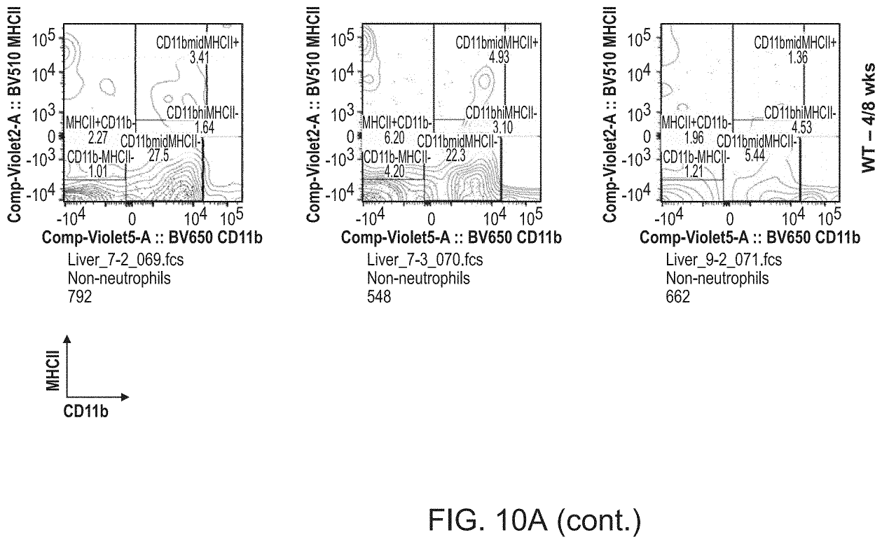

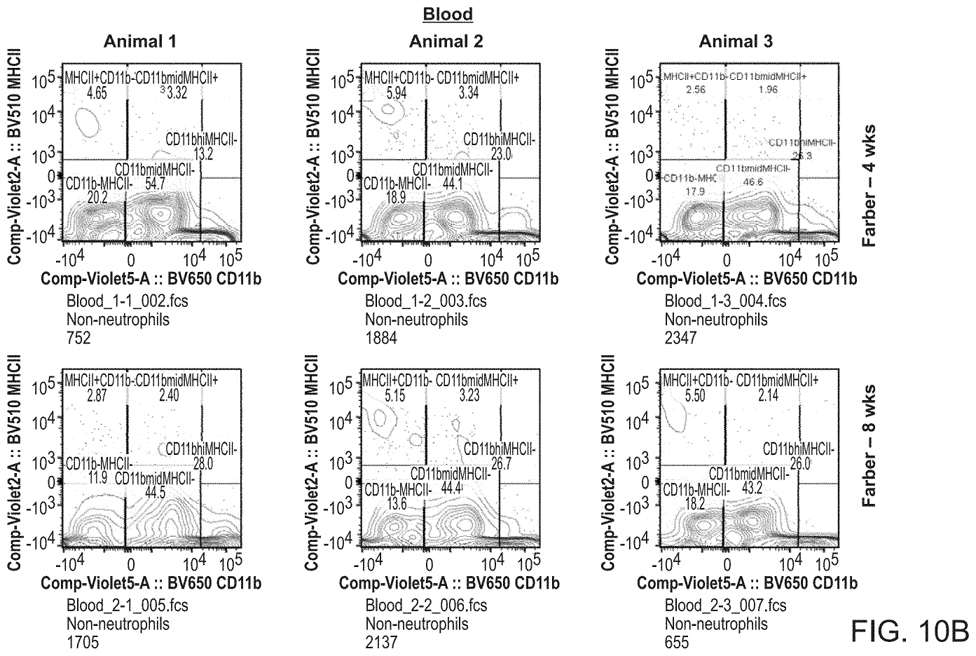

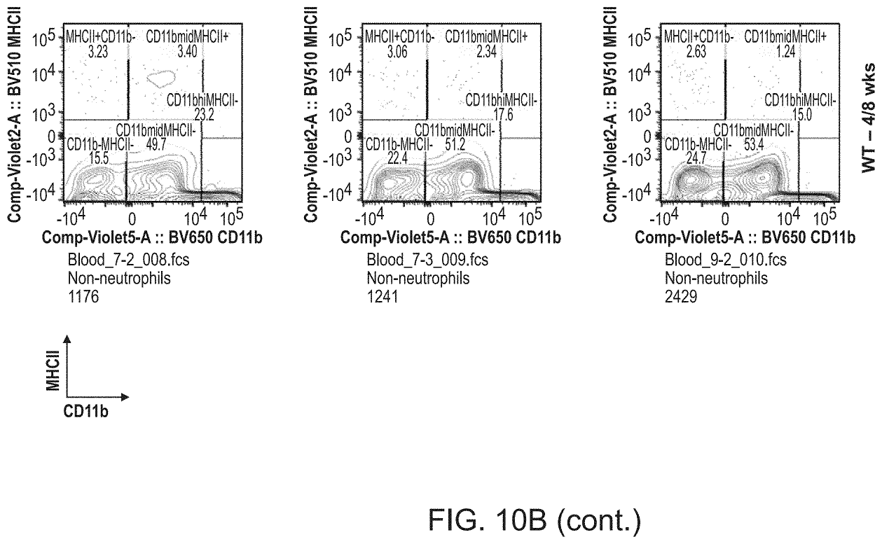

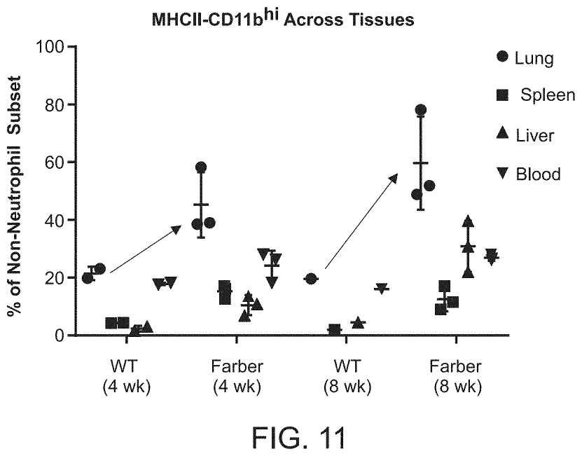

Monocytes/granulocytes are further divided into effector sub-populations. (Misharin et al, Am J Respir Cell Mol Biol. 2013 October; 49(4):503-10). Among the subsets, MHCII⁻CD11b^{hi} population are indicative of activated monocytes. As shown in FIG. 9A, marked increase in the MHCII⁻CD11b^{hi} population with concurrent decrease in MHCII⁻CD11b^{mid} were observed in Farber mice lung as compared to the wild type. Similarly, as shown in FIG. 9B, marked increase in the MHCII⁻CD11b^{hi} population were observed in Farber mice spleen as compared to the wild type (FIGS. 9B and 10A). This increased frequency in the tissue is also reflected in the blood (FIG. 10B). These findings of increased frequency of activated monocytes (MHCII⁻CD11b^{hi}) in the tissue and periphery of Farber mice (FIG. 11) support MHCII⁻CD11b^{hi} as an immune-phenotype marker for Farber disease.

MHCII⁺CD11b⁻Ly6C⁺(Pro-Inflammatory Monocyte Lineage)

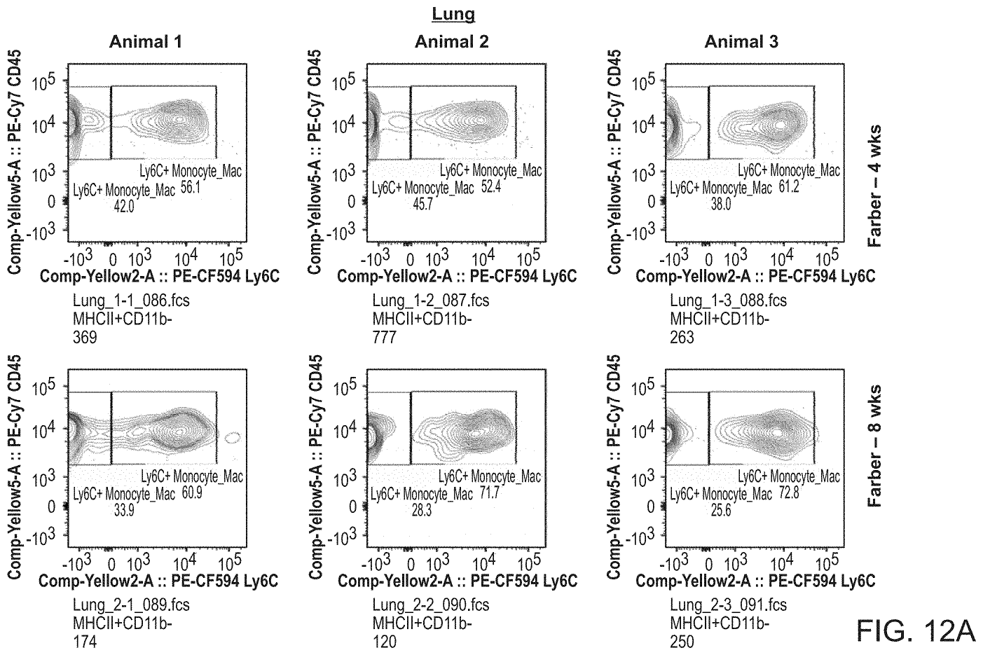

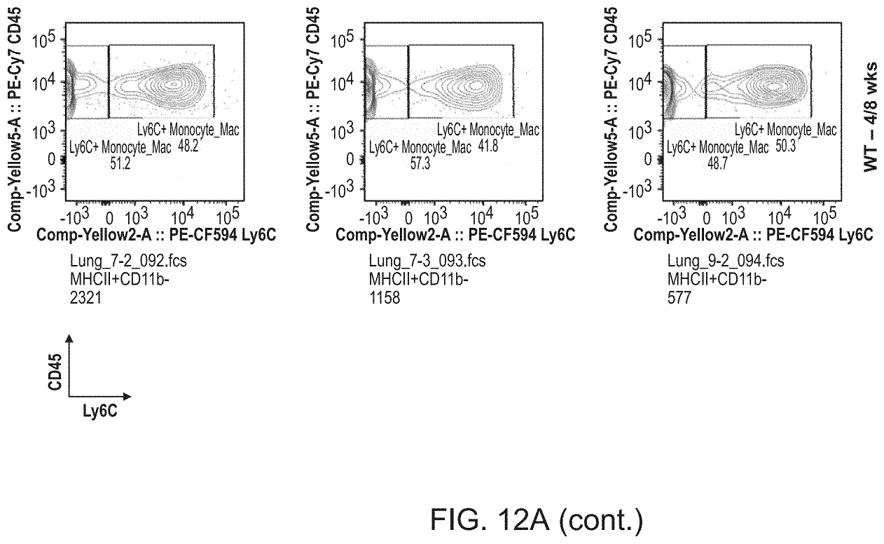

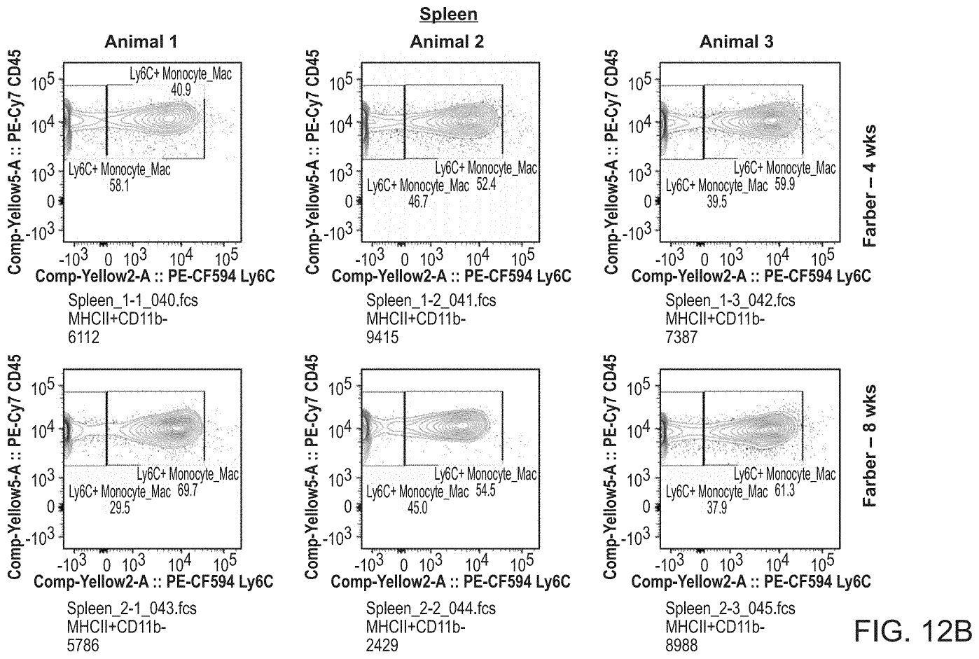

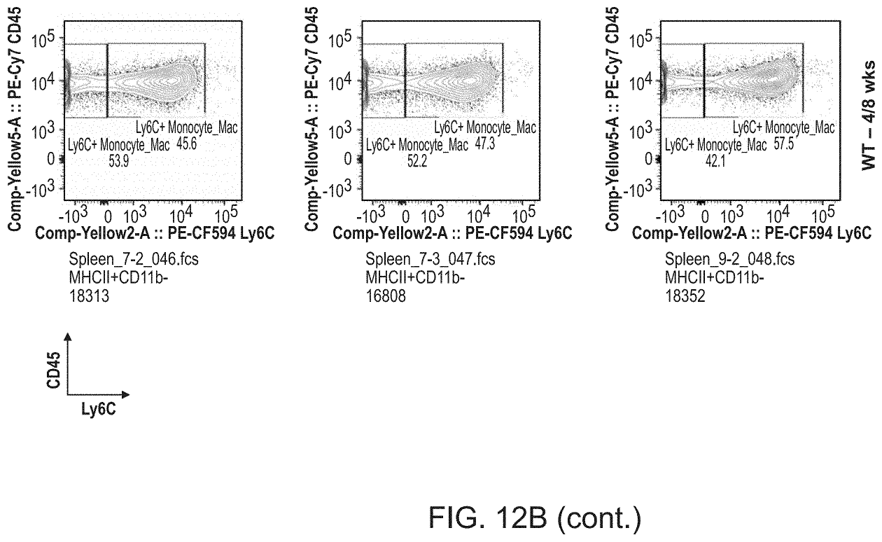

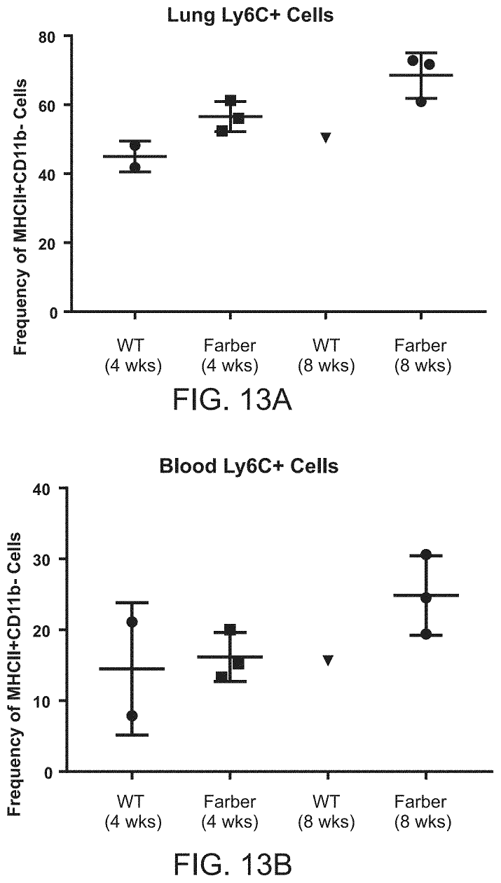

MHCII⁺CD11b⁻ can be further subdivided into Ly6C^{+/-}. Ly6C⁺ monocytes are more likely to differentiate into pro-inflammatory. Ly6C⁻ monocytes are more likely to differentiate into M2 macrophages and be anti-inflammatory. As shown in FIG. 13A, pro-inflammatory Ly6C⁺ sub-set of the MHCII⁺CD11b⁻ populations increased in the lung of Farber mice as compared to the wild-type mice. Similar Frequency of Ly6C⁺ Pro-Inflammatory Cells in the Spleen and Blood of Farber Mice. (FIGS. 12B and 13B). These findings show that inflammatory environment in situ can further promote the generation of pro-inflammatory cells. In Farber mice, this increase of pro-inflammatory macrophages and dendritic cells also was detected in the blood, supporting MHCII⁻CD11b^{hi}Ly6C⁺ as a peripheral marker for Farber disease.

MHCII⁻CD11b^{hi}, CD86⁺ (Activated Pro-Inflammatory Macrophages and Dendritic Cells)

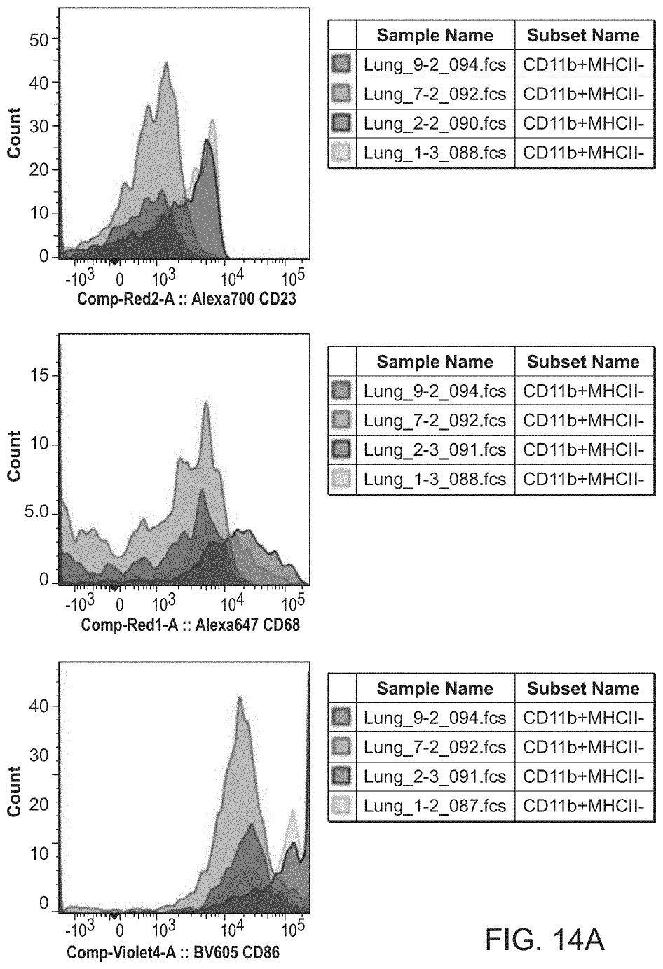

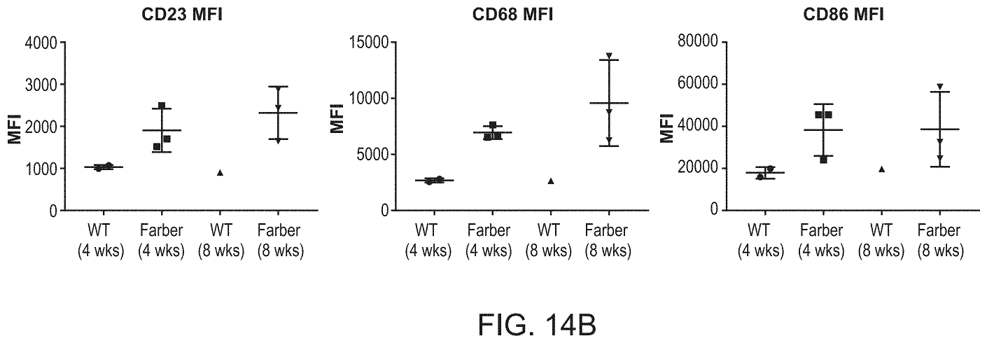

FIGS. 14A and 14B show CD23, CD68 and CD86 activation markers within the CD11b⁺MHC⁻DC population were increased in the Farber mice lung as compared to wild-type mice. In particular, increased expression of CD86 within the MHC⁻CD11b⁺ population in Farber mice (FIG. 14B, right panel) indicates the capacity to prime cells to induce immune cell recruitment and activation, perpetuating an inflammatory response. This result supports MHCII⁻CD11b^{hi} CD86⁺ as an immune-phenotype marker for Farber disease.

CD11b⁺CD38⁺ and CD11b⁺CD206⁺(Polarization of Macrophages)

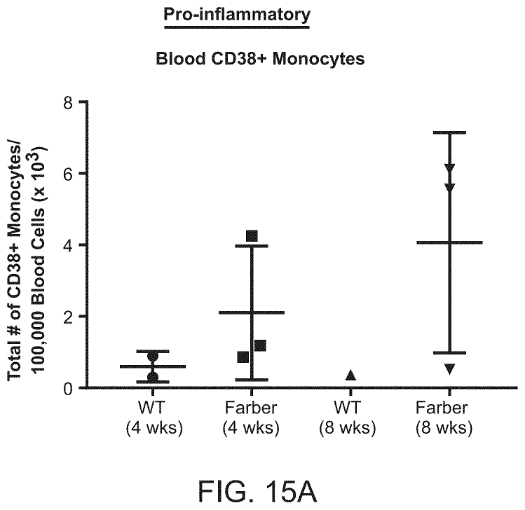

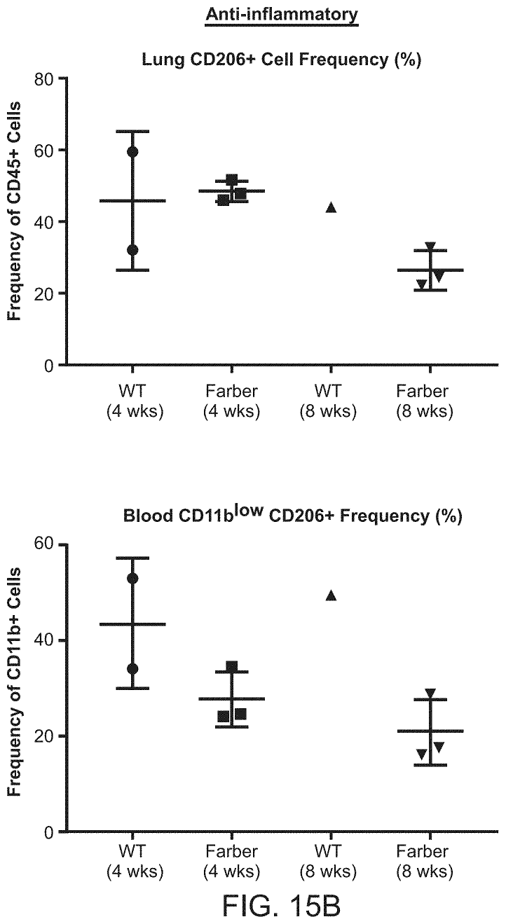

Farber mice have an increase in pro-inflammatory macrophages and a decrease in anti-inflammatory macrophages (FIGS. 15A and 15B). Polarization of macrophages is regulated by cytokine milieu and nutrient source. Cytokines/chemokines are cell signaling molecules that can drive chemotaxis and induce cellular changes in target cells.

CD11b⁺Ly6G⁺ (Neutrophil)

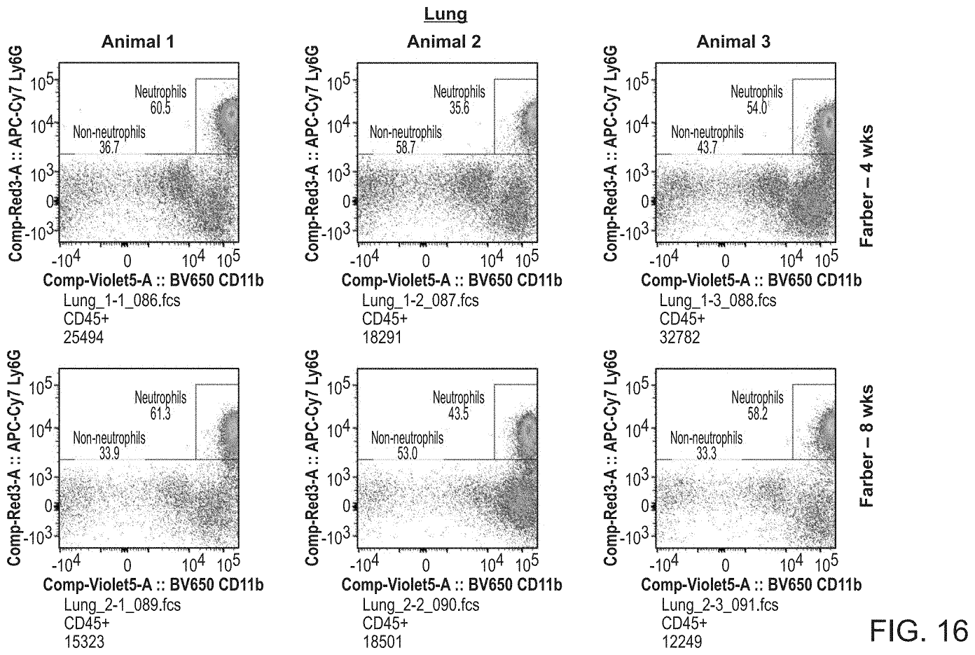

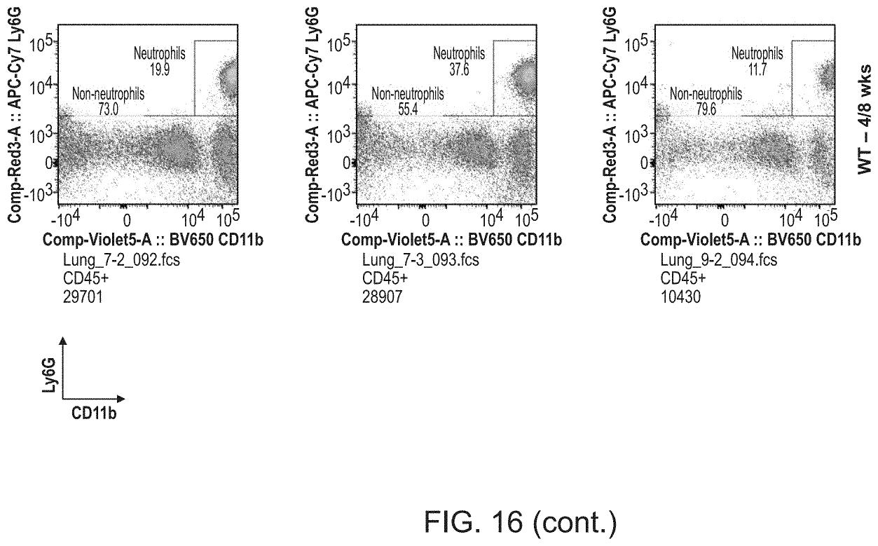

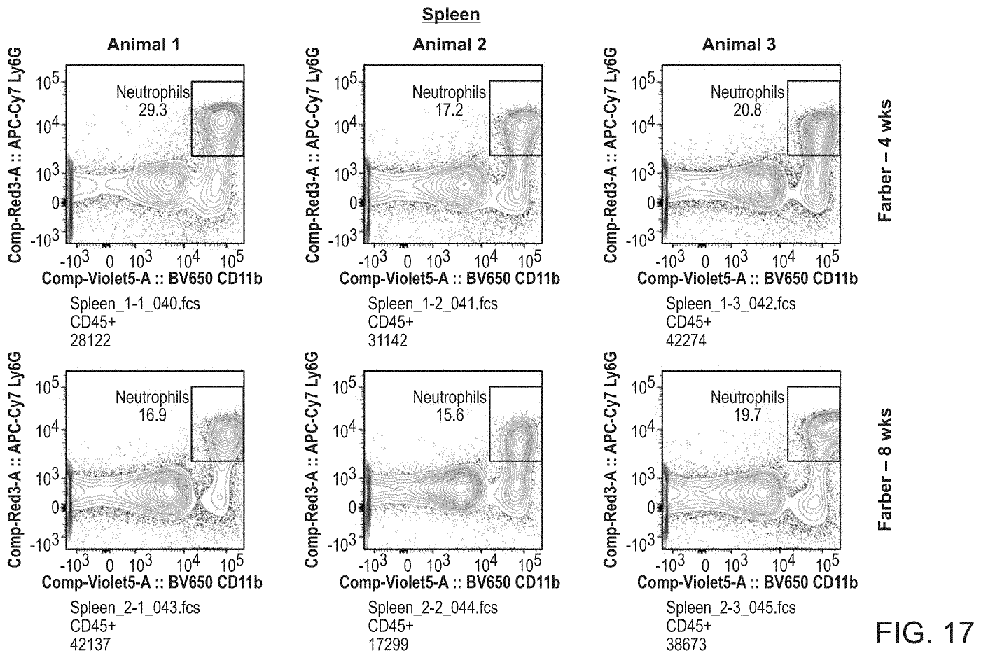

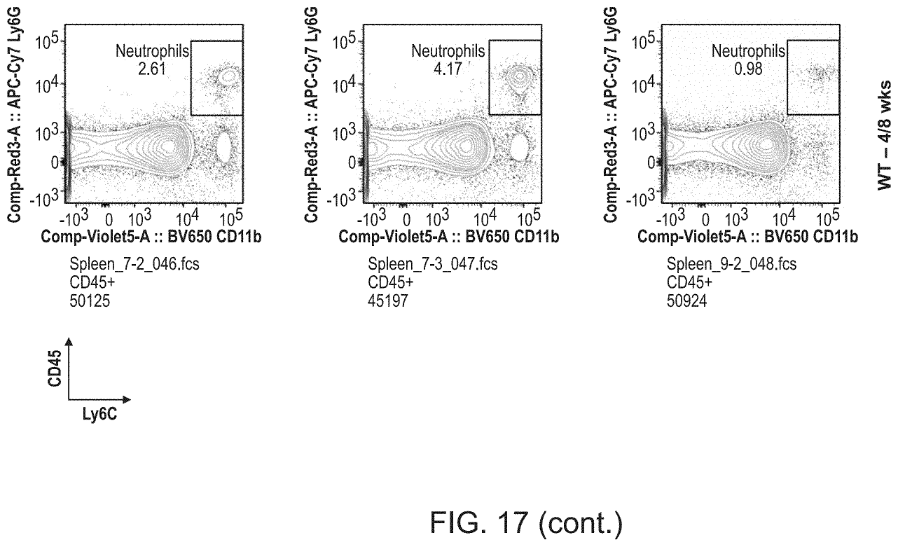

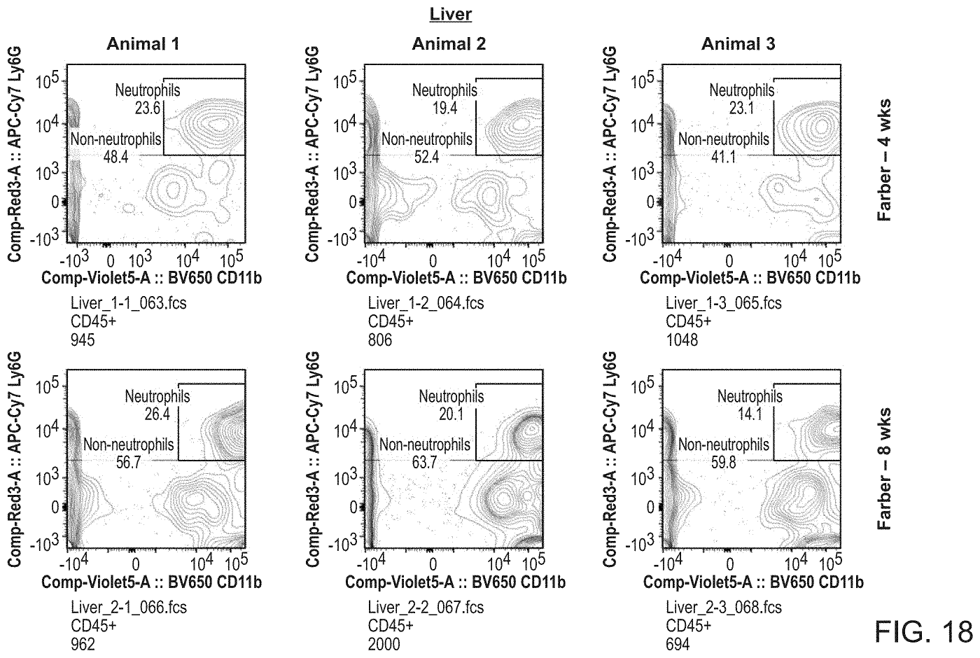

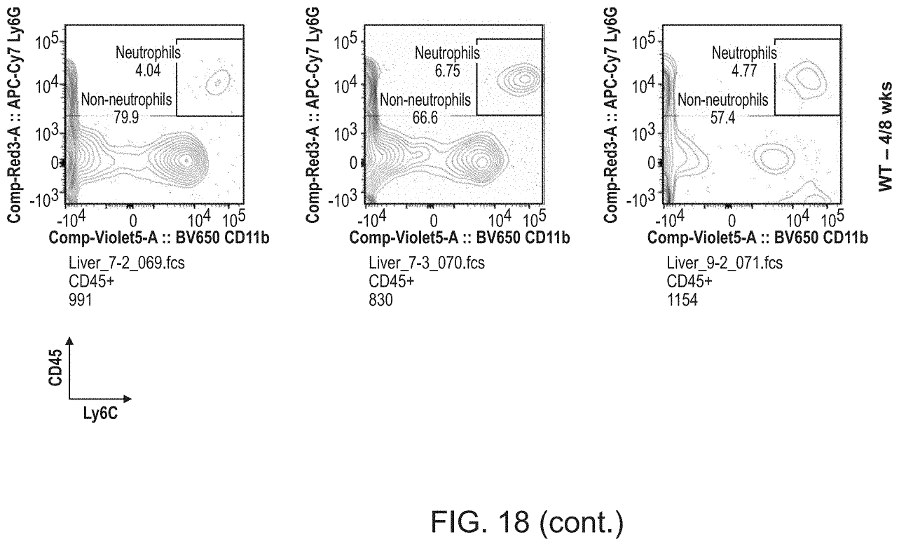

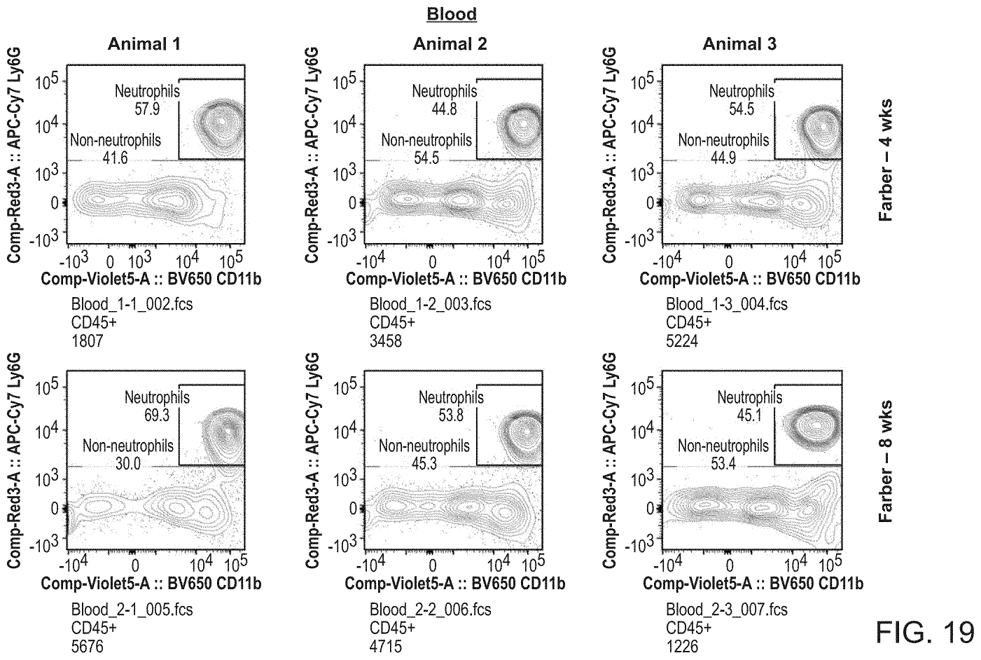

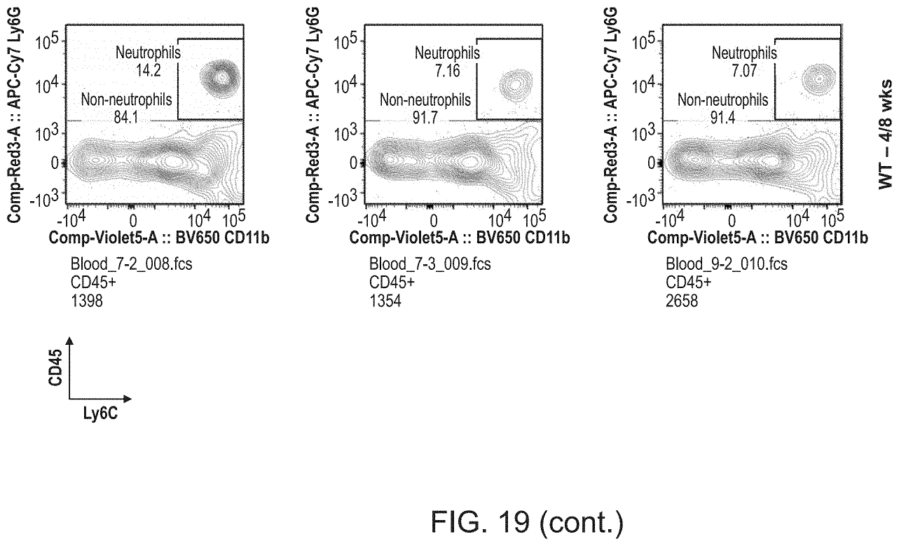

As shown in FIGS. 16-19 and 20A-20D, all of the lung, spleen, and liver of Farber mice showed increased frequency of neutrophils (CD11b⁺Ly6G⁺) by 4 weeks of age (2-7 fold increase compared to the wild type). The increase in neutrophils was the most remarkable differentiating factor between Farber mice and wild type mice across tissues (see Table 8 below). An early and robust increase in neutrophil infiltration may help drive subsequent immune response including monocyte recruitment, and thus contribute to pathology in Farber mice via production of pro-inflammatory cytokines.

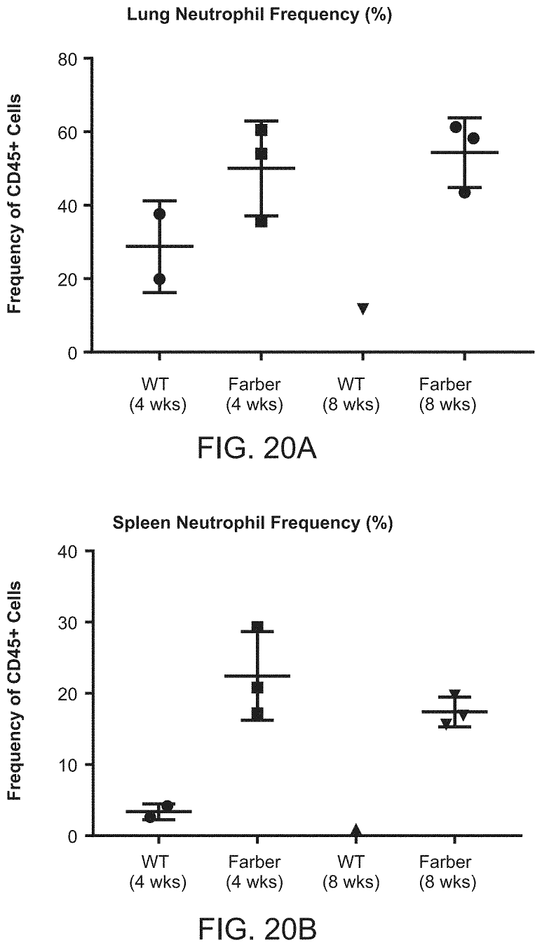

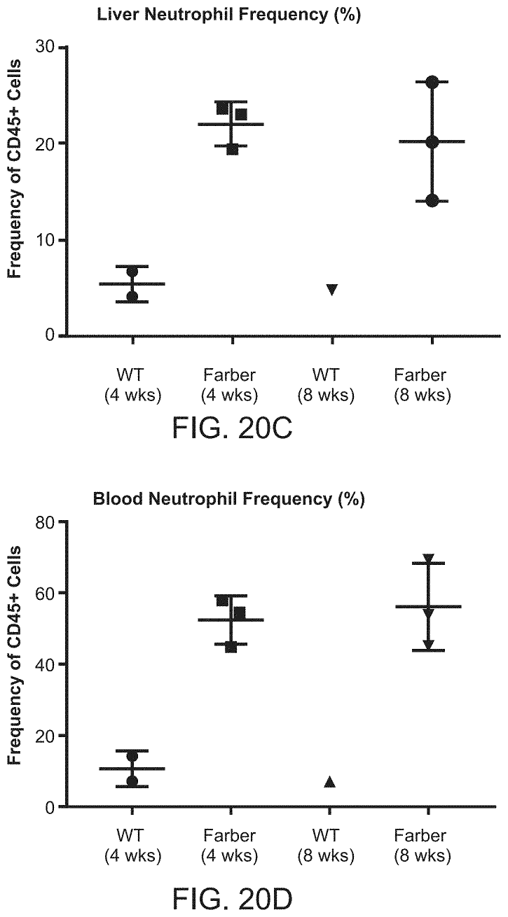

Further, as shown in FIG. 20D, increase in tissue-resident neutrophil frequency was also reflected in the blood of Farber mice, supporting CD11b⁺Ly6G⁺ as a strong phenotype marker for Farber disease.

CD19b⁻CD3⁺ (T Cells)

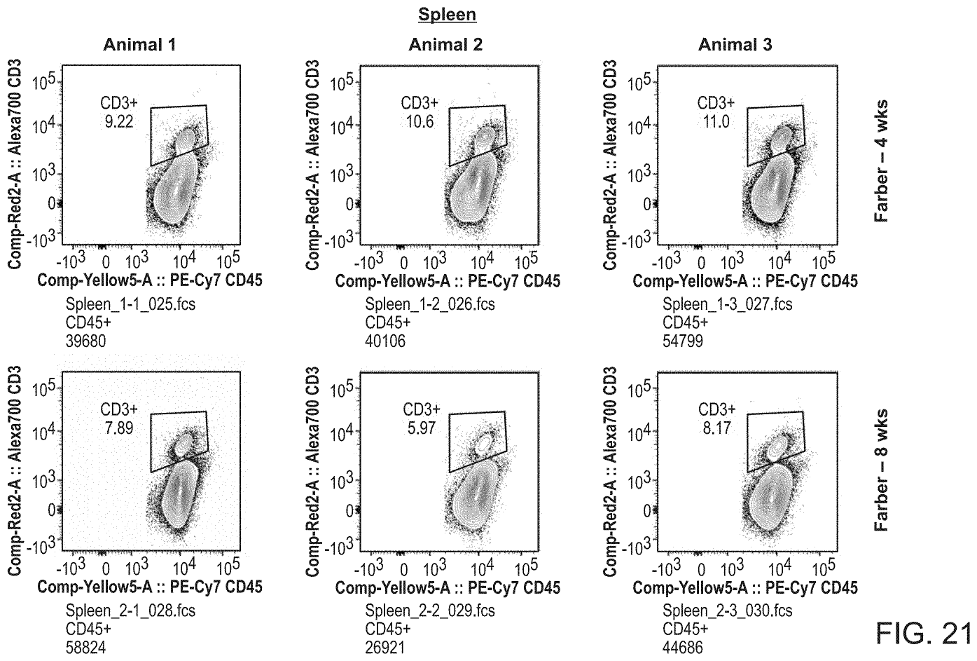

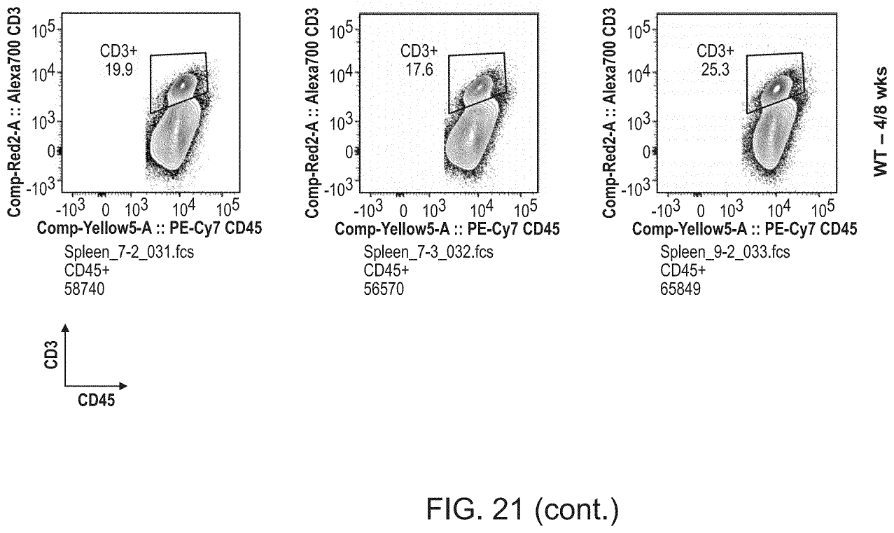

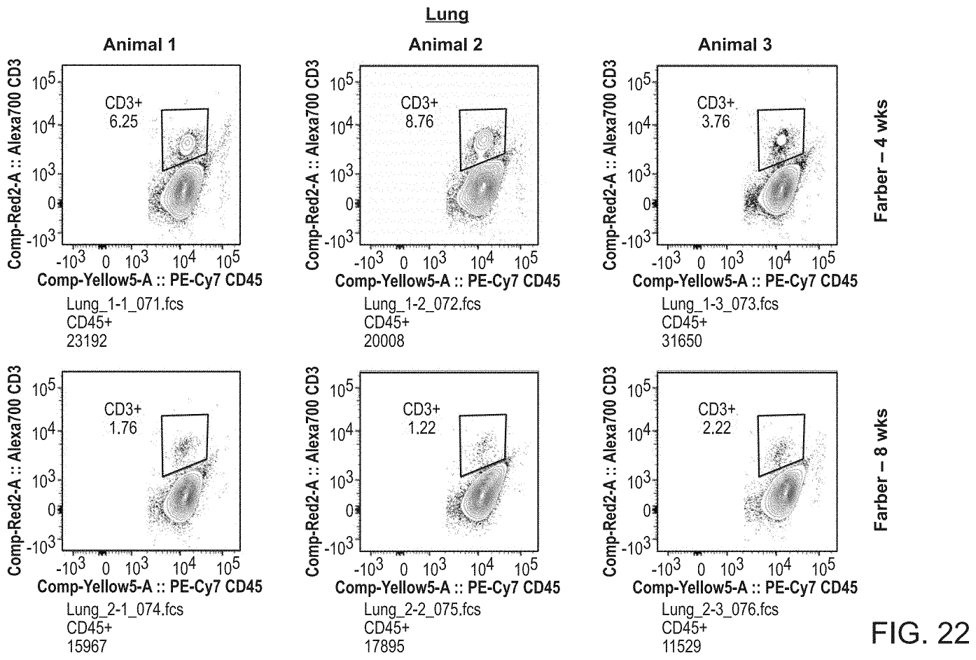

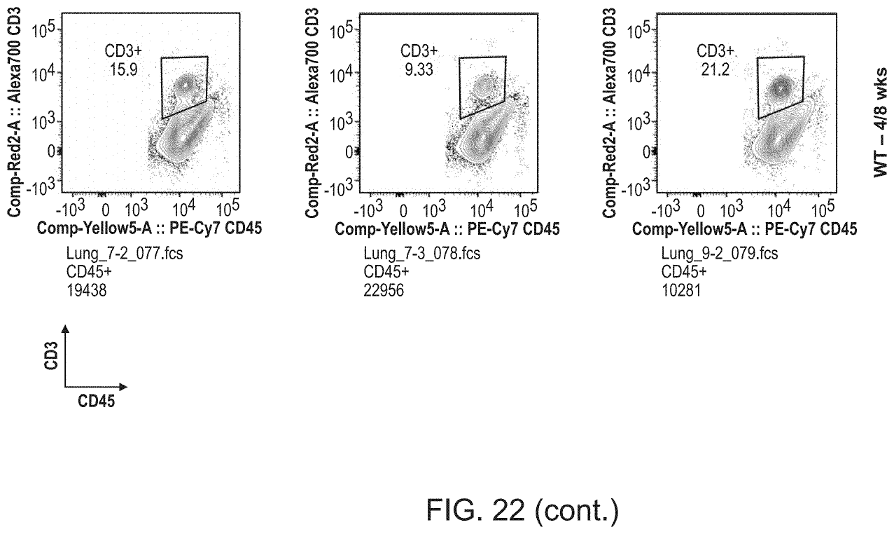

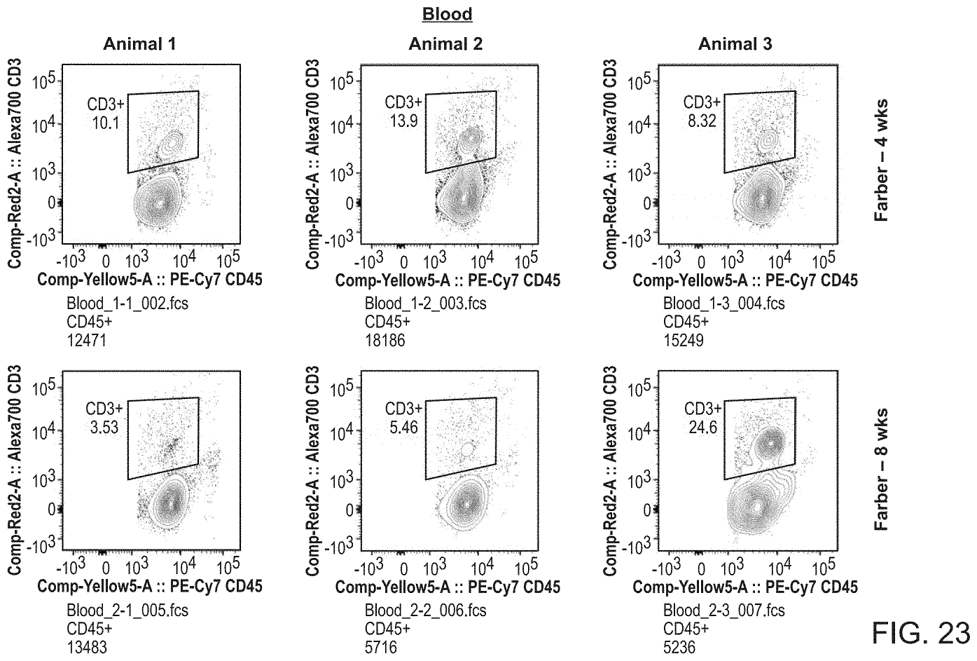

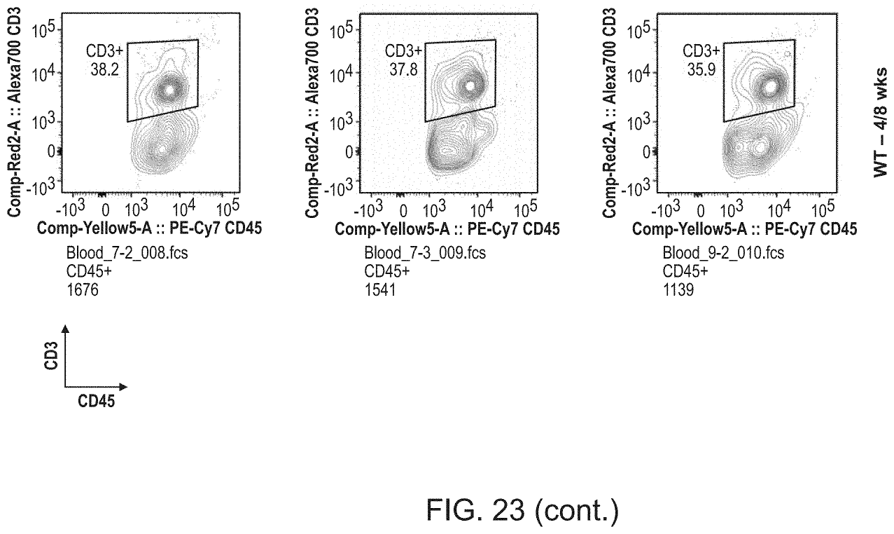

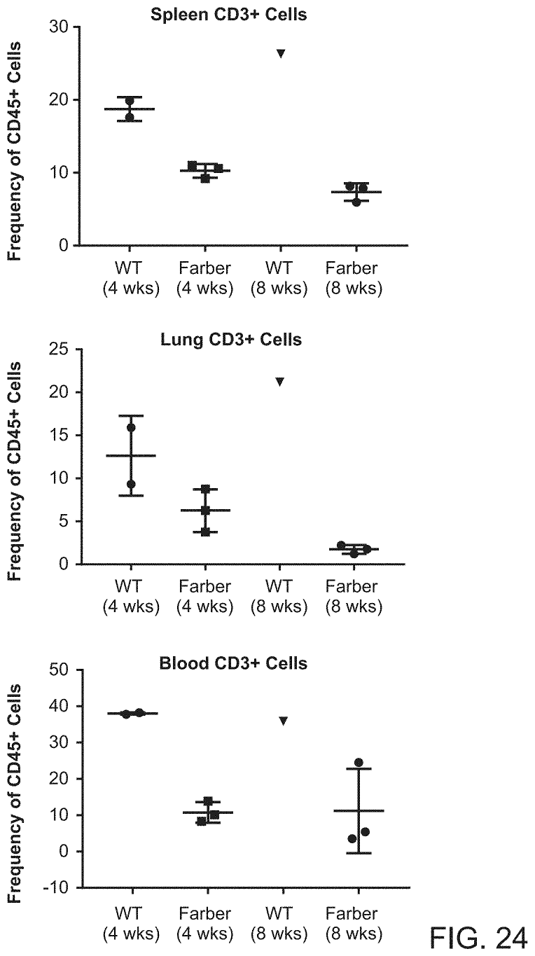

As shown in FIGS. 21-24, all of the spleen, lung, and blood of Farber mice showed decreased frequency of T cells (CD19b⁻CD3⁺) at both 4 weeks and 8 weeks (FIG. 24). This finding is consistent with the previous observation of substantial destruction of the thymus structure and decrease of thymic T cells in Farber mice (Dworski et al., 2015), and sphingosine 1-phosphate dependence in the regulation of lymphocyte development and migration into the intestines. (Kunisawa et al., 2007.)

CD19b⁺CD38⁺ (Activated B Cells)

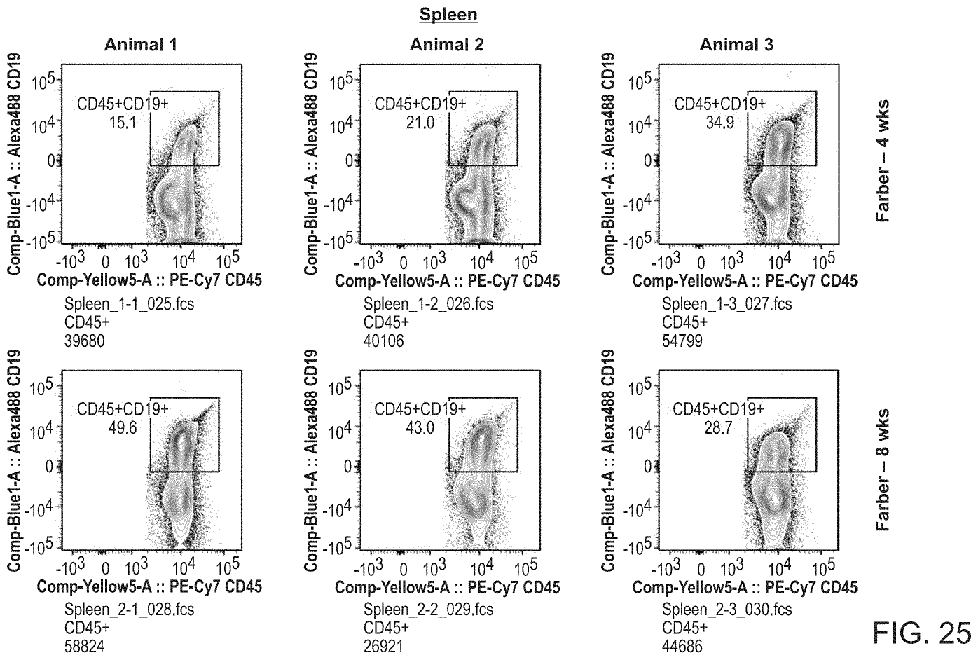

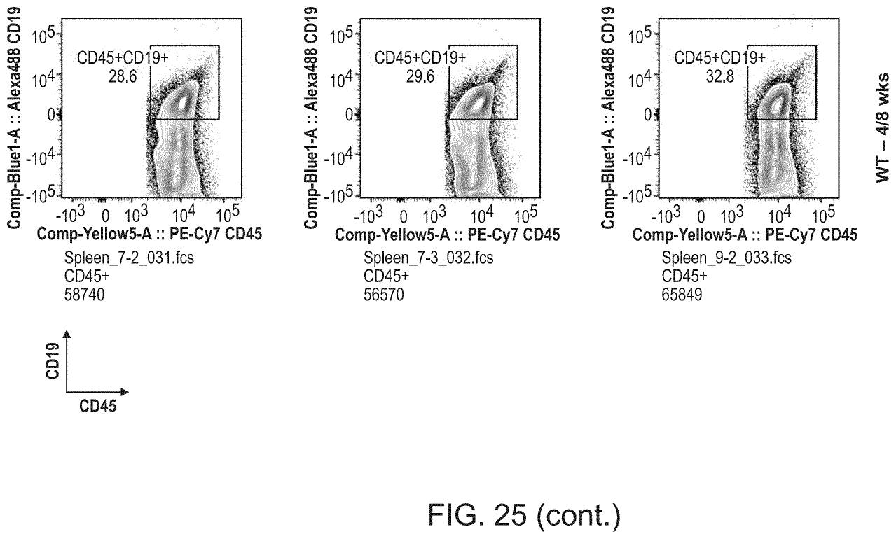

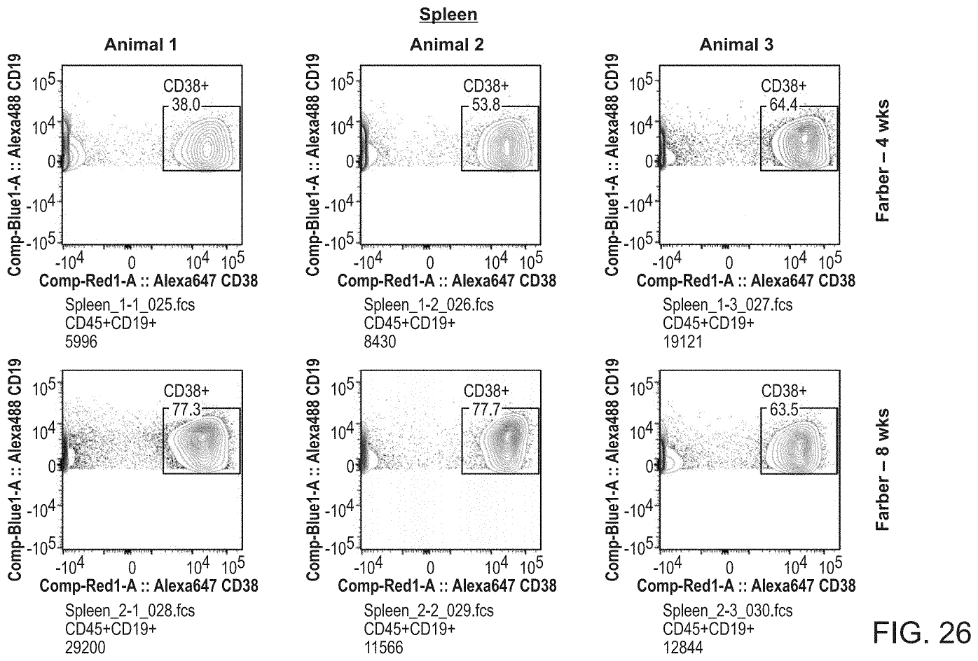

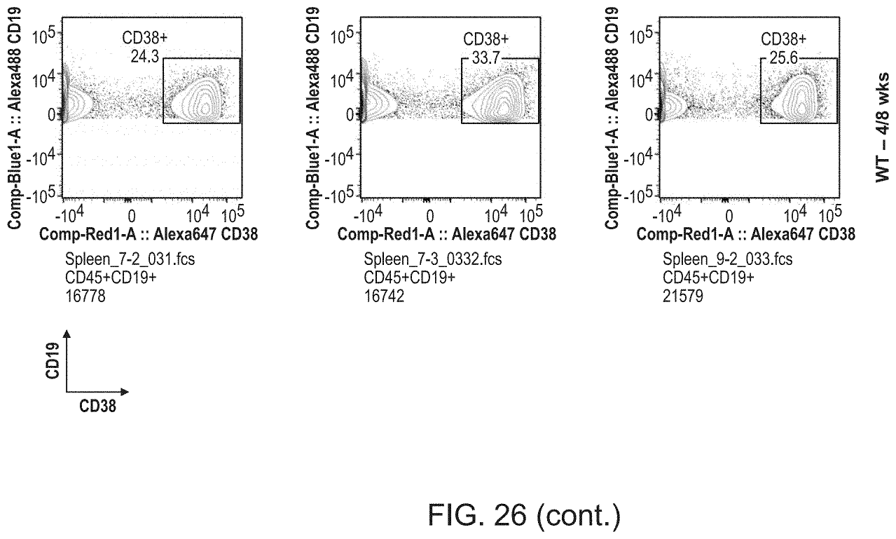

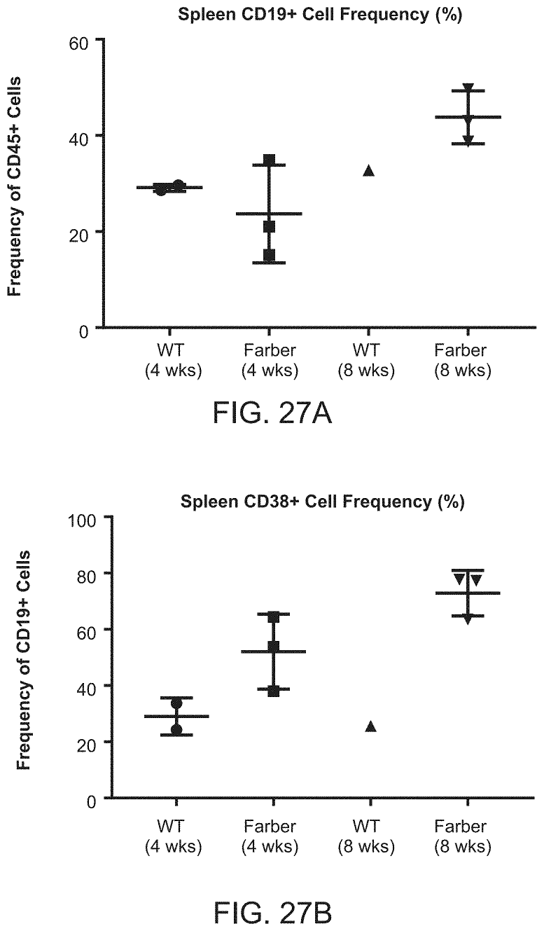

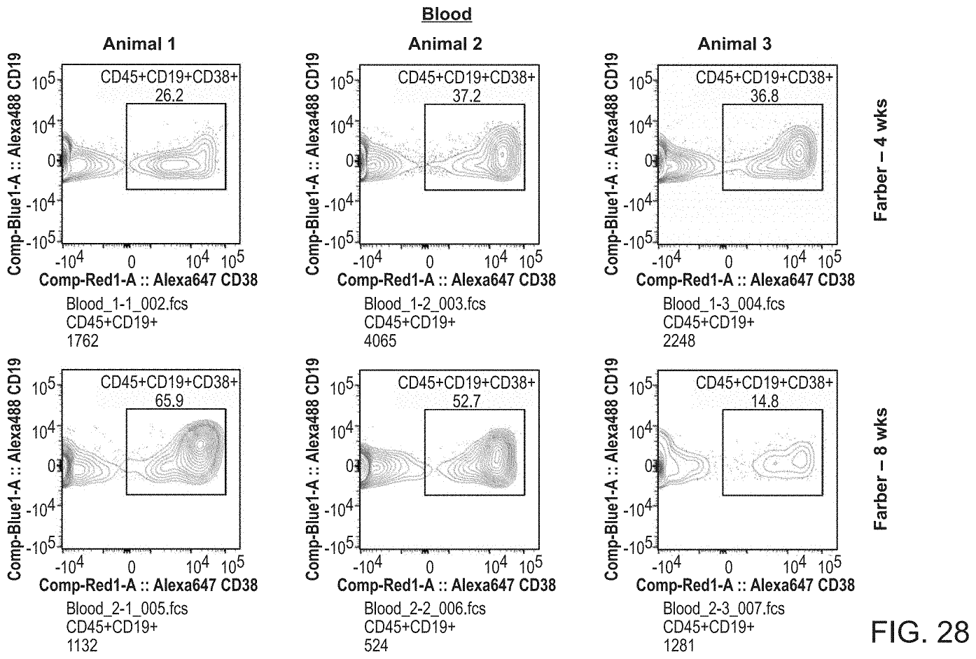

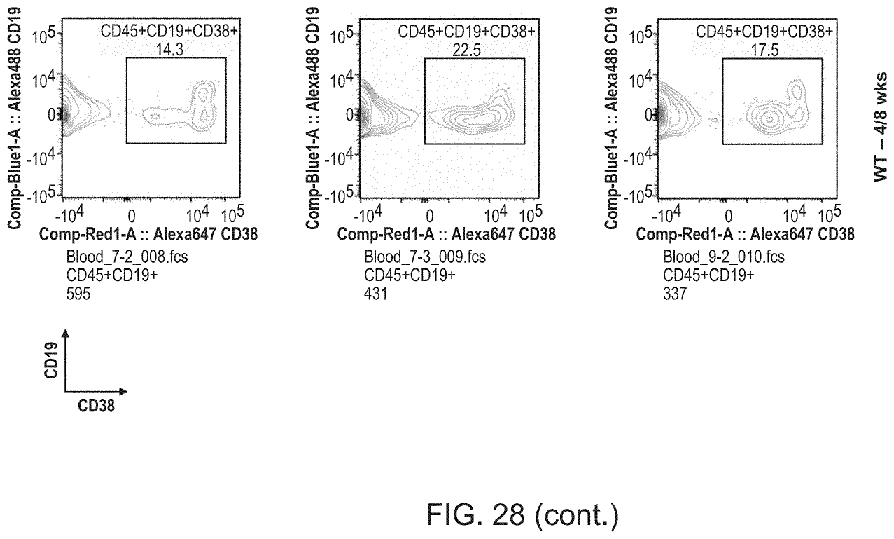

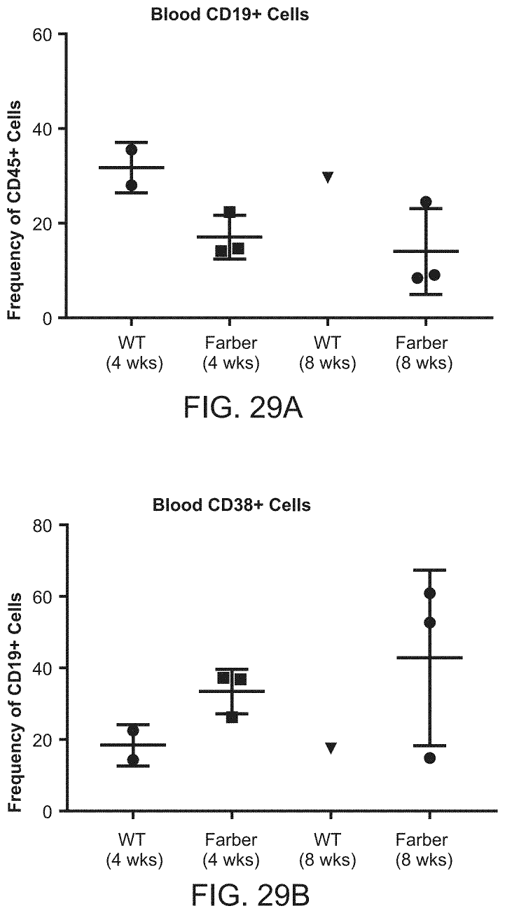

As shown in FIGS. 25 and 27A, Farber Mice Have an Increased Frequency of B Cells in the Spleen as evidenced by CD19⁺. Within the B cell compartment in the spleen, Farber mice had an increased frequency of activated B Cells (plasmablasts) as evidenced by CD38⁺ (FIGS. 26 and 27B). Increased Frequency of Activated B Cells were reflected in the periphery as shown in FIGS. 28, 29A, and 29B. The decrease in T cells is likely related to thymus destruction whereas the increase in activated B cells is likely due to an abundance of antigen presenting cells. Both T cell and B cell kinetics suggest that response is secondary to monocyte (and derived subpopulations) and neutrophil infiltration and activation.

CD45^{hi}SS^{hi} (Eosinophils or Basophils)

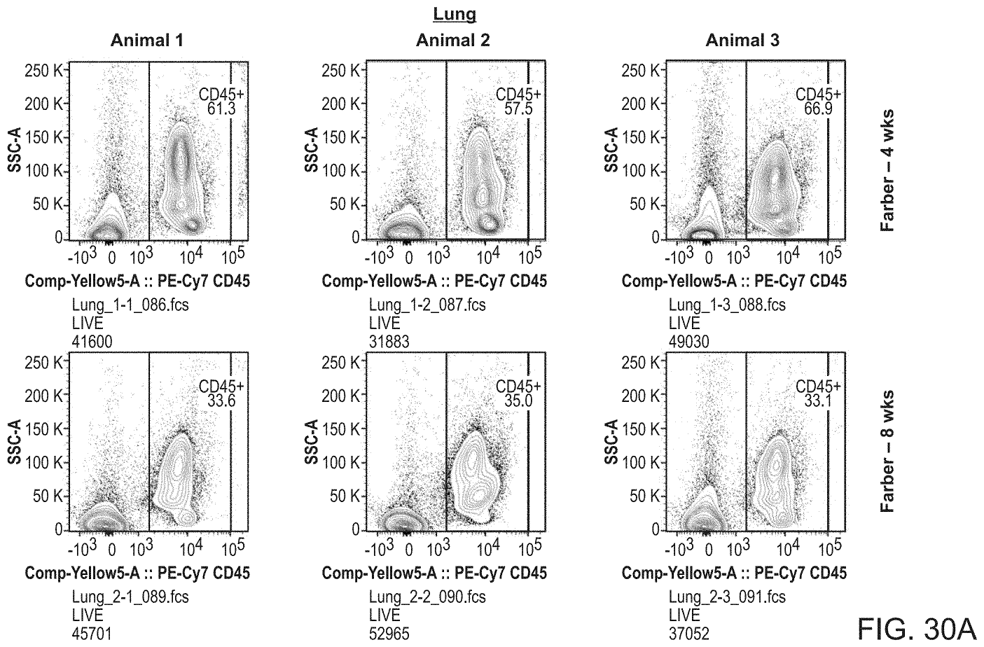

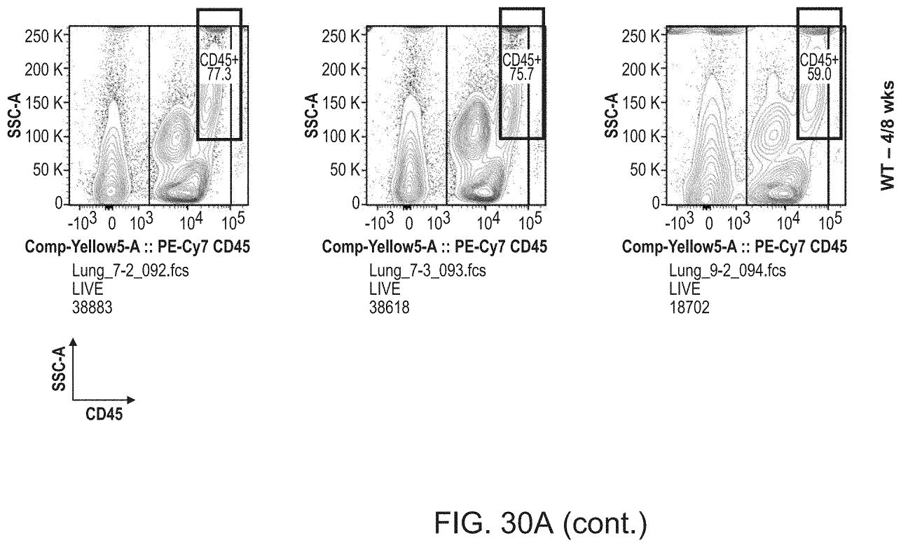

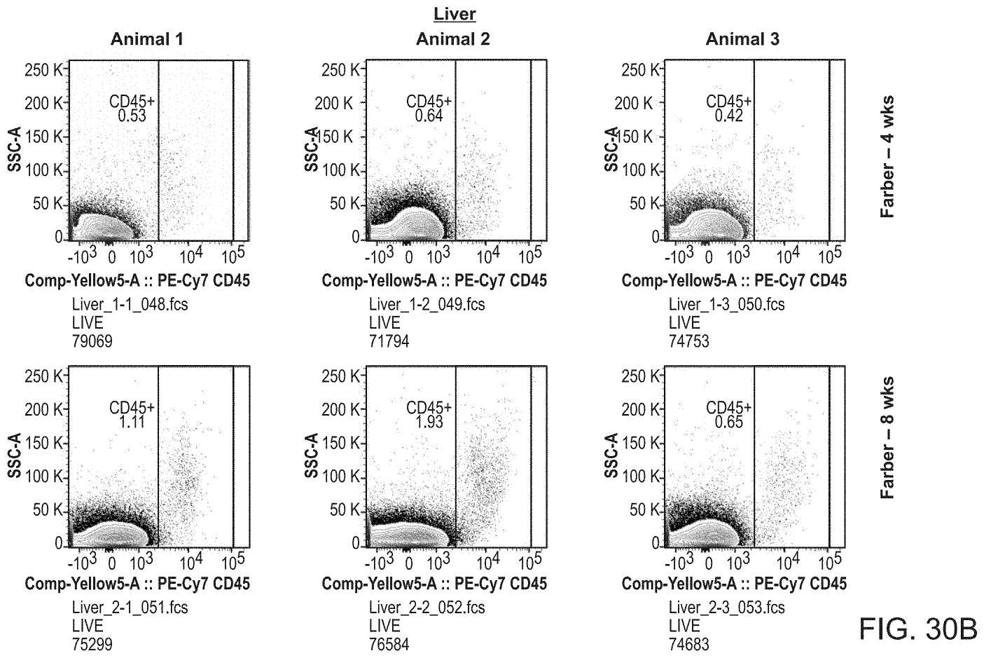

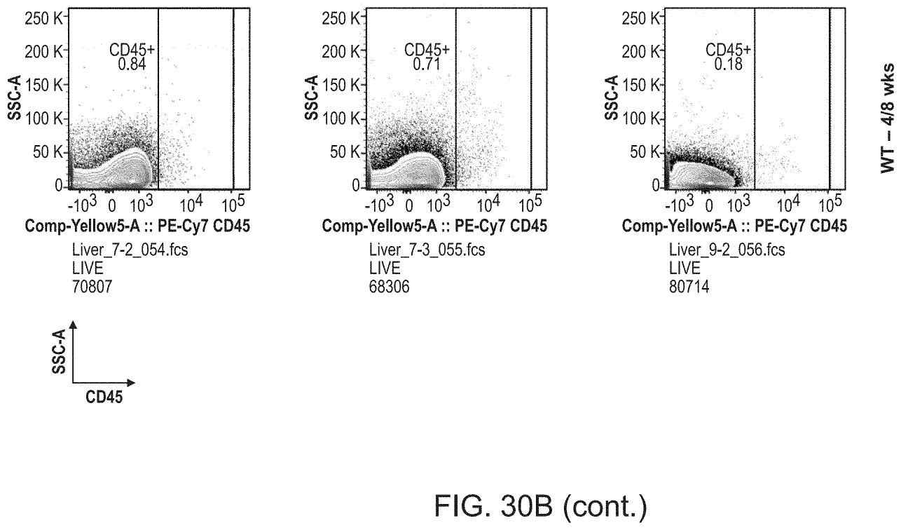

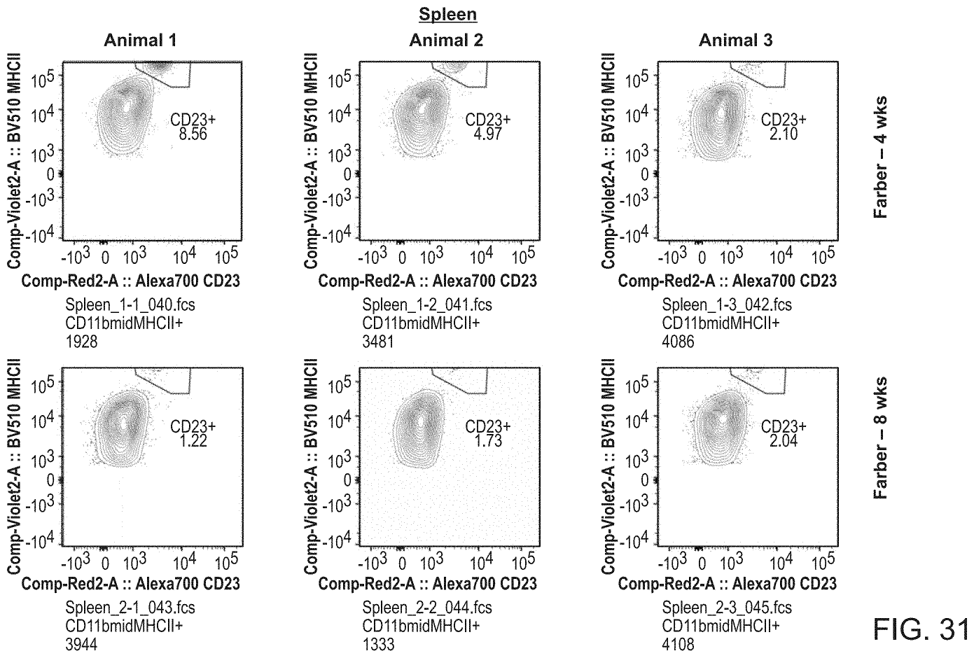

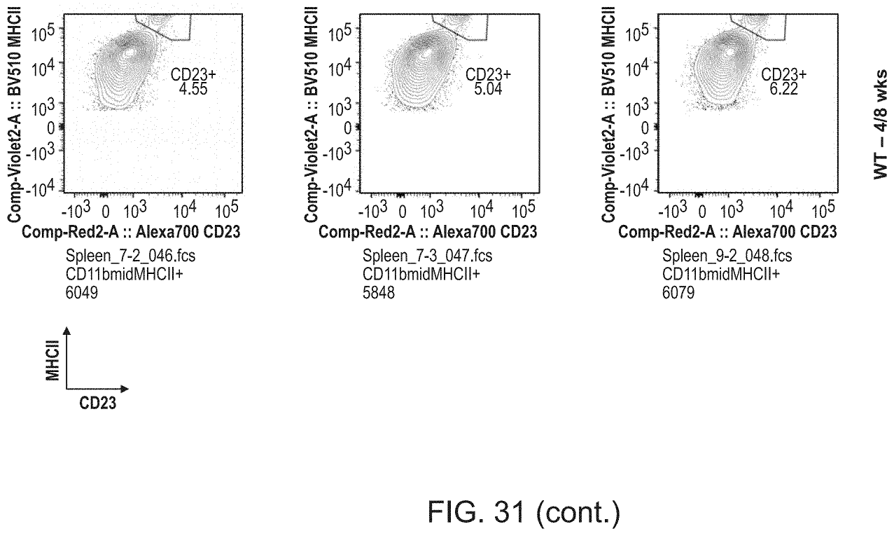

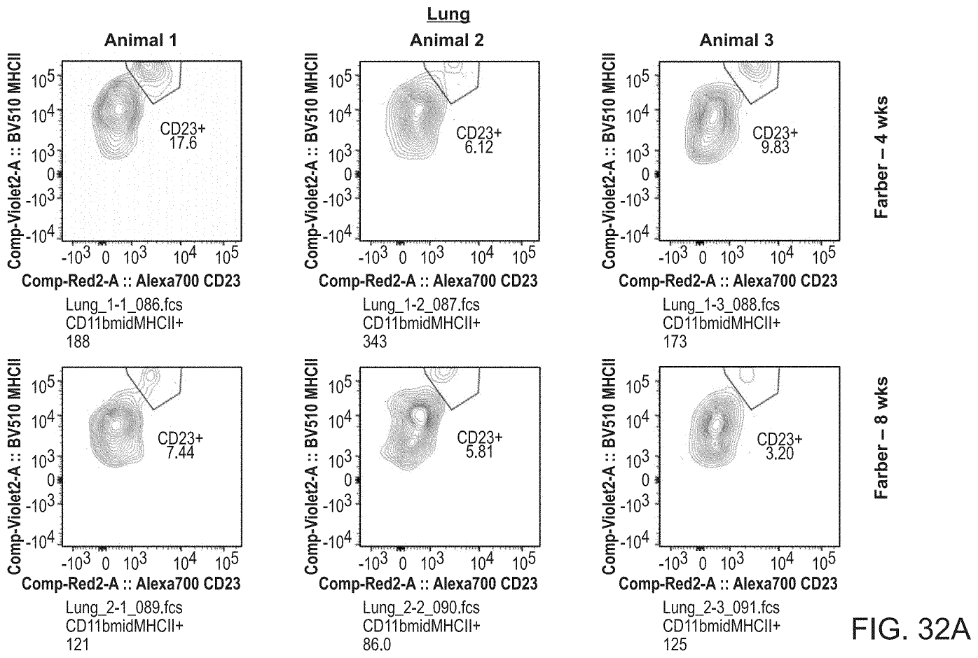

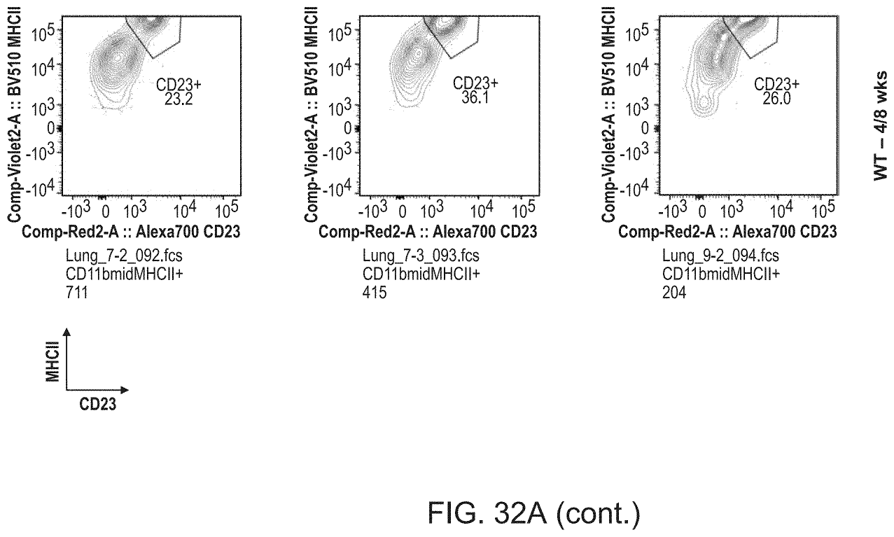

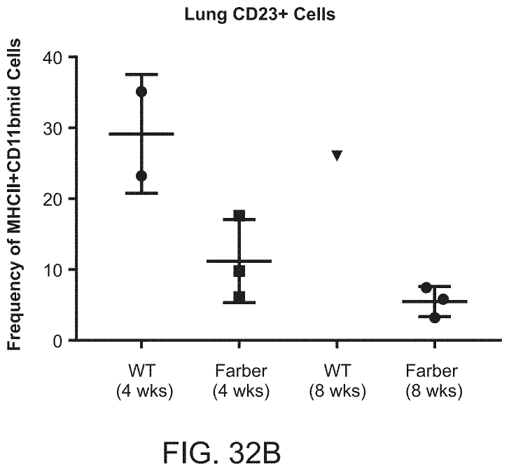

In Farber mice lung, the overall frequency of immune cells was decreased compared to wild type mice at either age (FIG. 30A). In particular, CD45^{hi}SS^{hi} cells (FIG. 30A, black outlines) completely disappeared in the Farber mice.

In Farber mice liver, the frequency of immune Cells was generally low in both Farber and wild type mice (FIG. 30B). The liver has a complex histological structure, which consists primarily of hepatocytes (CD45′) 70% of hepatic cellular component), with intrahepatic lymphocytes (IHL) constituting 16-22% of the remaining nonparenchymal cells (30%). CD45⁺SS^{hi} cells in 8-week old Farber mice was slightly increased compared to 4-week old Farber mice and WT mice at either age.

MHCII⁺CD11b^{mid}CD23⁺

CD23 is expressed on mature B cells, activated macrophages, eosinophils, follicular dendritic cells, and platelets. Loss of CD23⁺ cells is indicative of activation in Farber Mice spleen and lung. No CD23 was found in the blood sample.

TABLE 8
Changes assessed against 8 weeks old Farber mice vs. wild type littermates.

| Set of markers | Cell Type | Lung | Spleen | Liver | Blood | Cumulative |
|---|---|---|---|---|---|---|
| CD11b⁺Ly6G⁺ | Neutrophil | 5 | 5 | 2-5 | 5 | +++ |
| SSC^{mid}FSC^{mid} | Bulk Monocytes | — | 5 | 5 | 5 | +++ |
| (Size) | | | | | | |
| MHCII⁻ | Activated Monocytes | 2-5 | 5 | 5 | 1-2 | +++ |
| CD11b^{hi} | | | | | | |
| CD11b⁺CD206⁺ | Anti-inflammatory | (1-2) | — | — | (5) | (++) |
| | mΦ & DCs | | | | | |
| MEICII⁺CD11b⁻ | Pro-inflammatory | 1-2 | — | — | 2-5 | + |
| Ly6C⁺ | mΦ & DCs | | | | | |
| MHCII⁻ | Activated | — | — | (1-2) | — | (+) |
| CD11b^{mid} | Pro-inflammatory | | | | | |
| CD23⁺ | mΦ & DCs | | | | | |
| MHCII⁻ | Activated | 1-2 | — | — | 1-2 | + |
| CD11b^{hi} | Pro-inflammatory | | | | | |
| CD86⁺ | mΦ & DCs | | | | | |
| CD11b⁺CD38⁺ | Pro-inflammatory | 1-2 | 2-5 | — | 5 | +++ |
| | mΦ & DCs | | | | | |
| CD19⁺CD38⁺ | Activated B Cells (PBs) | — | 2-5 | — | 2-5 | ++ |
| CD19⁻CD3⁺ | Total T Cells | (5) | (2-5) | — | (2-5) | (+++) |

Table 8 provides the summary of the markers identified for diagnosing the Farber disease. Compared to the control samples from the age-matched wild-type mice, “1-2” indicates 1-2 fold change in marker expression, “2-5” indicates 2-5 fold change, “>5” indicates greater than 5-fold change, and “-” indicates “not tested or not determined.” The “Cumulative” score is a sum of the marker expression in all four tissue and blood samples, and “+++” indicates greater than 5-fold change, “++” indicates 3 to 4-fold change, and “+” indicates less than 3-fold change. Parentheses indicate a negative change or decrease compared to the wild type control.

Characterization of the monocyte population revealed a marked increase in the frequency of MHCII⁻CD11b^{hi}Ly6C⁺ cells. These cells were highly activated as evidenced by CD86 expression, and skewed toward a pro-inflammatory M1 phenotype, based on CD38 expression. A concurrent decrease in CD206⁺ anti-inflammatory M2 macrophages was identified. In addition to the macrophage compartment, a profound increase in neutrophils in the spleen, liver and lung was evident by 4 weeks of age (2-7 fold vs. wild-type). Marked differences in the adaptive immune compartment also were noted, with a clear increase in the frequency of plasmablasts (precursors to Immunoglobulin-producing plasma cells), likely secondary to the increase in pro-inflammatory monocytes.

Immune-Fingerprint

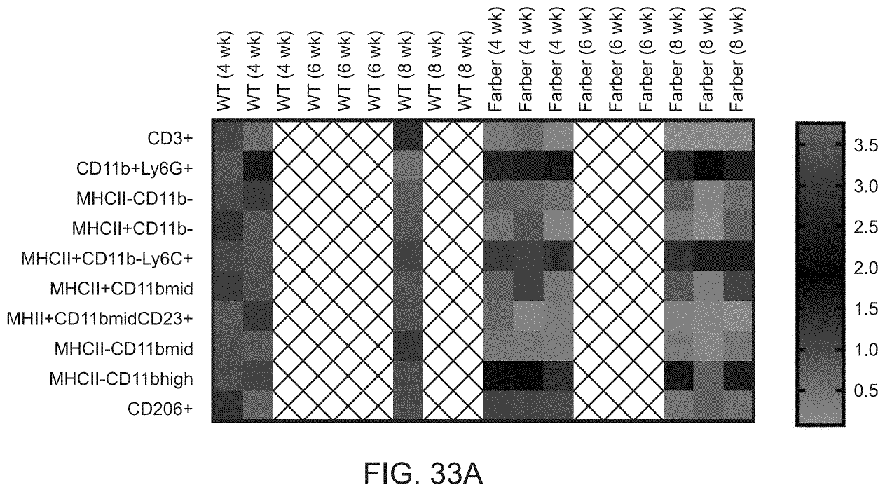

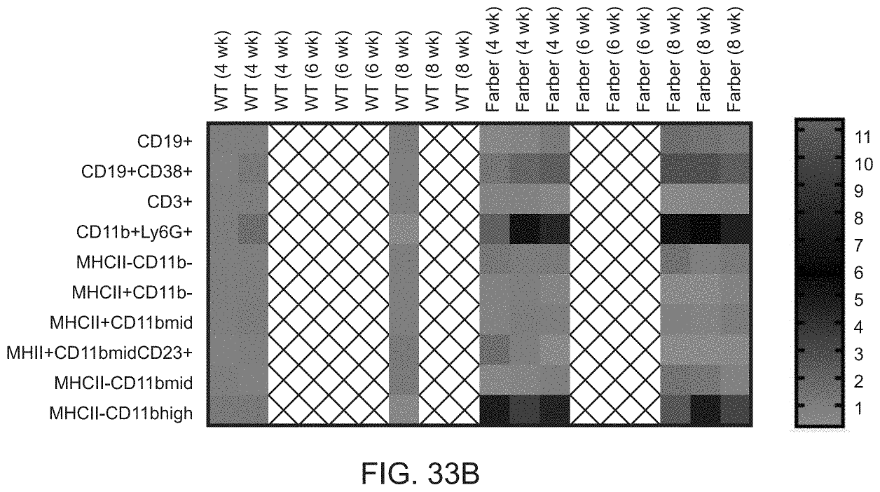

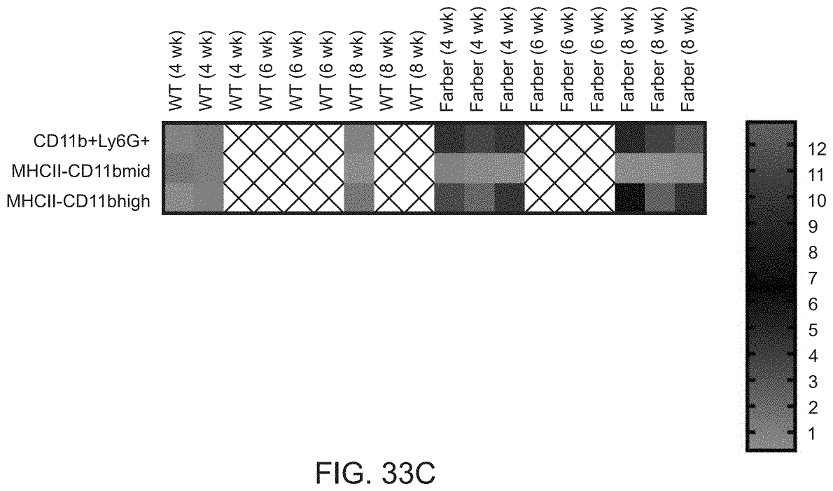

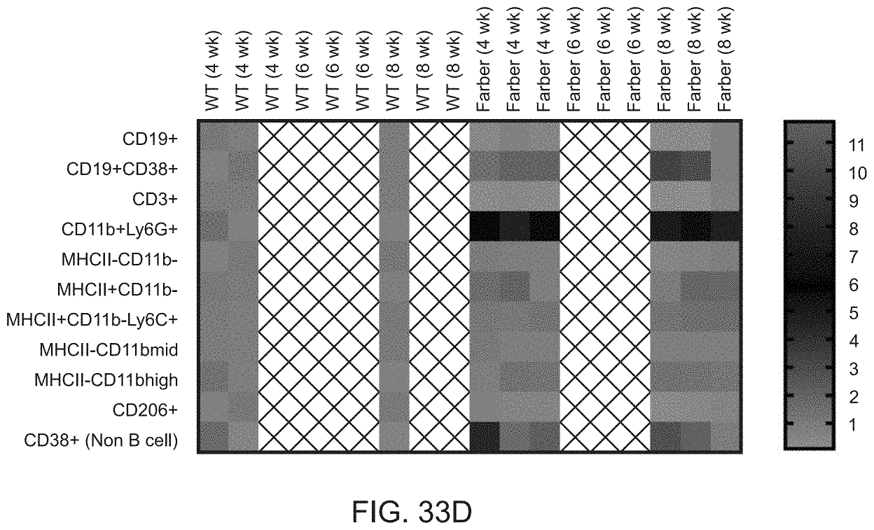

FIGS. 33A-33D show examples of immune-fingerprint based on all subsets identified in of Farber mice lung, spleen, liver and blood.

Further studies will be conducted to delineate the effects of rhAC on immune-phenotypes of tissues of Farber mice treated with rhAC. Tissues can be stained with the above-identified markers used to diagnose Farber disease listed in Tables 2, 3, and 7, including pro-inflammatory markers (e.g., CD38, Ly6G) and anti-inflammatory markers (e.g., CD206) and pan-monocyte markers (e.g., CD11b).

### Example 2

Wild type, Farber mouse, and Farber mouse treated with a recombinant human acid ceramidase (RVT-801) were analyzed for immune cell population makeup. The Farber mouse model was used, as it is a “knock-in” mouse model established on a W4/129Sv/CD-1 background with a single nucleotide missense mutation identified in a severe-onset FD patient to create a homozygous Asah1^{P361R/P361R} animal that produced a non-functional version of acid ceramidase. This disease model recapitulates monocytic infiltration of multiple tissues and is therefore useful to study the immune environment of Farber disease using this diseases model.

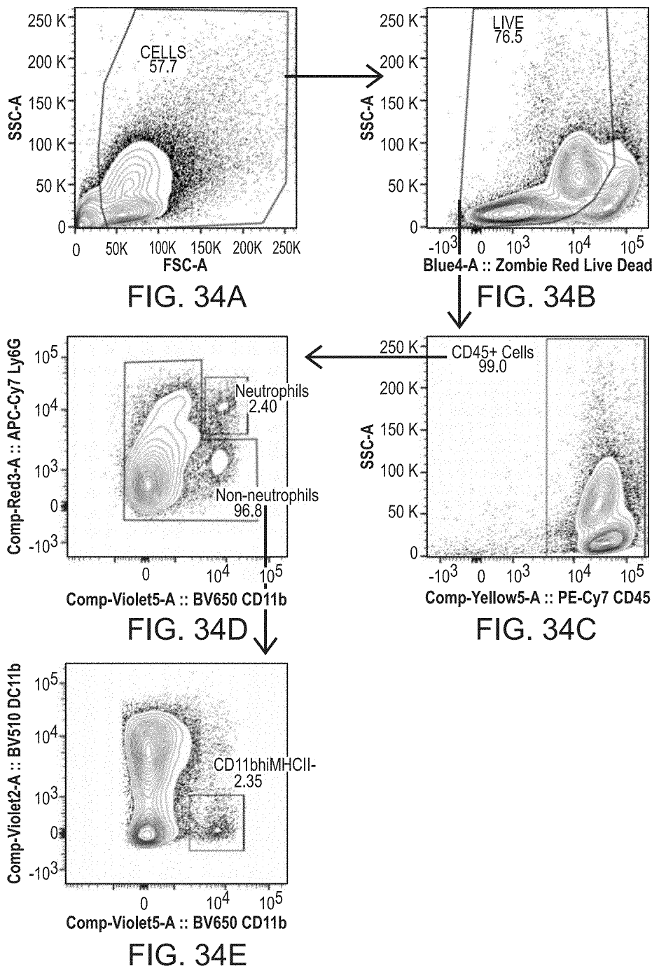

Farber mice (genotype confirmed by PCR) were dosed with 4-once weekly intraperitoneal (IP) doses of 10 mg/kg/dose recombinant human acid ceramidase (RVT-801) beginning just after weaning (aged 3-4 weeks) and were sacrificed for necropsy following their 4th and final RVT-801 administration (at 7 weeks of age). Control wild type and Farber mice were not dosed with vehicle and three control animals of each genotype were necropsied and samples collected for assessment at 4 or 8 weeks of age. At the indicated time points, Farber mice and littermate controls (WT) were harvested to assess the composition of immune cells in key tissues of ceramide accumulation (spleen, liver and lung). In addition, blood was collected to correlate the tissue-specific inflammation in the periphery. Samples were processed to a single cell suspension, stained according to Table 9 (Panel 1) and Table 10 (Panel 2) described below, and run on a BD LSRII flow cytometer. Single stained samples (compensation beads) and fluorescence minus one (FMO) samples served as controls for the study. Raw data files were analyzed using FlowJo v10 and a representative gating strategy is shown as FIGS. 34A-E.

Results. FIG. 34A-E cell populations that were first gated based on size (SSC×FSC) to remove cellular debris from processing (FIG. 34A). This population was further gated based on live and dead cells to remove the cell population that was positive for the Zombie red dye (FIG. 34B). The live cells were then gated to select the CD45⁺ population (FIG. 34C). This population was further gated to determine the percent of CD45⁺ cells that were Ly6G and CD11b double positive; or neutrophils (FIG. 34D). The remaining population was selected and gated to select for the CD11b⁺MHCII⁻ population to determine the population of activated monocytes per sample type (FIG. 34E). This was done for whole blood, spleen, liver, and lung samples in this way using FlowJo v10. Lung samples were further gated to select for activated macrophages.

TABLE 9
Neutrophil Panel

| Marker | Clone | Fluorophore |
|---|---|---|
| CCR2 | SA203G11 | BV421 |
| CD68 | FA-11 | Alexa647 |
| CD11c | N418 | BV711 |
| MHCII | M5/114.15.2 | BV510 |
| CD86 | GL-1 | BV605 |
| CD206 | C068C2 | Alexa488 |
| CD11b | M1/70 | BV650 |
| CD45 | 104 | PE-Cy7 |
| Ly6G | 1A8 | APC-Cy7 |
| Ly6C | HK1.4 | PE-CF594 |
| CX3CR1 | SA011F11 | PE |
| F4/80 | BM8 | PE-Cy5 |
| CD23 | B3B4 | Alexa700 |

TABLE 10
Monocyte Panel

| Marker | Clone | Fluorophore |
|---|---|---|
| CD3 | 500A2 | Alexa700 |
| CD19 | 6D5 | Alexa488 |
| CD4 | GK1.5 | APC-Cy7 |
| CD8 | 43-6.7 | BV650 |
| CD62L | MEL-14 | BV605 |
| CD44 | IM7 | PE |
| CD45 | 104 | PE-Cy7 |
| CCR7 | 4B12 | PE-Cy5 |
| CD127 | A7R34 | BV711 |
| MHCII | M5/114.15.2 | BV510 |
| CD38 | 90 | Alexa647 |
| CCR6 | BV421 | 29-2L17 |
| CXCR3 | CXCR3-173 | PE-CF594 |

### Example 3

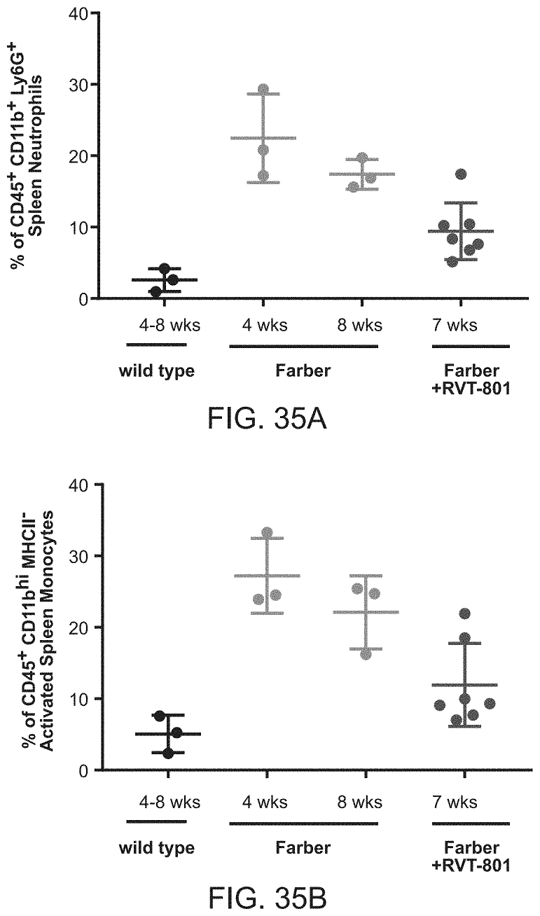

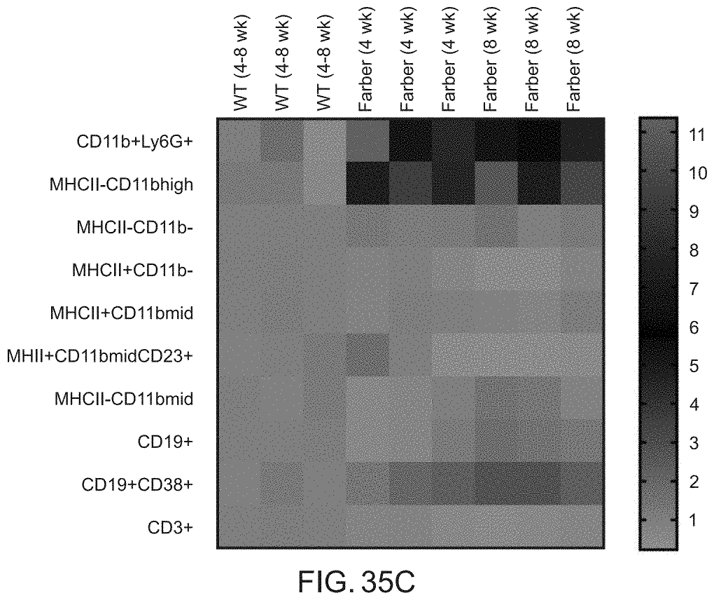

Splenic immune cell populations. Results are depicted in FIGS. 35A and B. Inflammatory cell populations which are characteristic of an inflammatory state were analyzed from control 4 and 8 week old wild-type and Farber mouse spleens. The population of Ly6GCD11b double positive CD45⁺neutrophils and CD11b⁺MHCII⁻CD45⁺ activated monocytes were determined from 7 week old Farber mice that were administered 10 mg/kg/dose RVT-801 once weekly beginning at 3 weeks of age for a total of 4 doses over 4 weeks. Heat map analysis of fold change differences in the frequency of immune cells in the spleen of age-matched Farber mice and littermate controls (WT). Each column represents an individual animal. Fold change was calculated by setting the average WT value to 1.

### Example 4

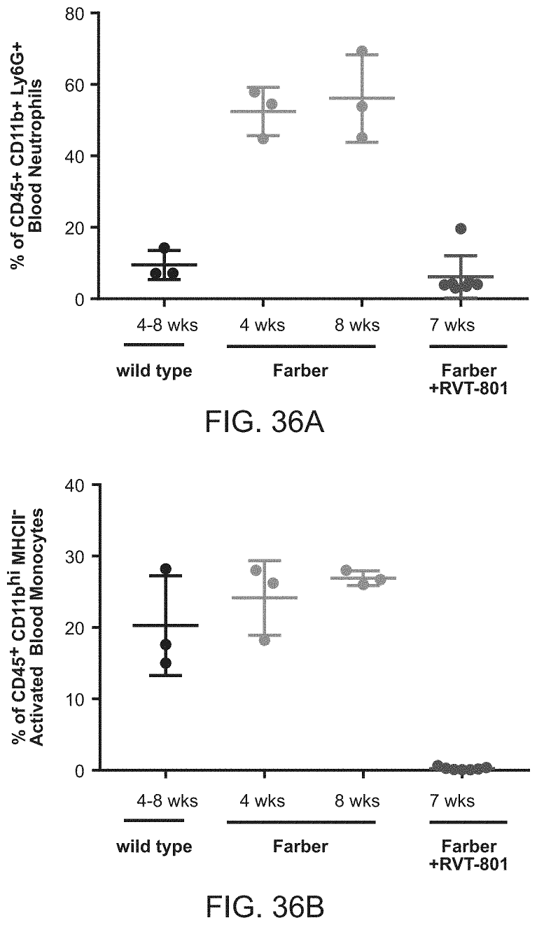

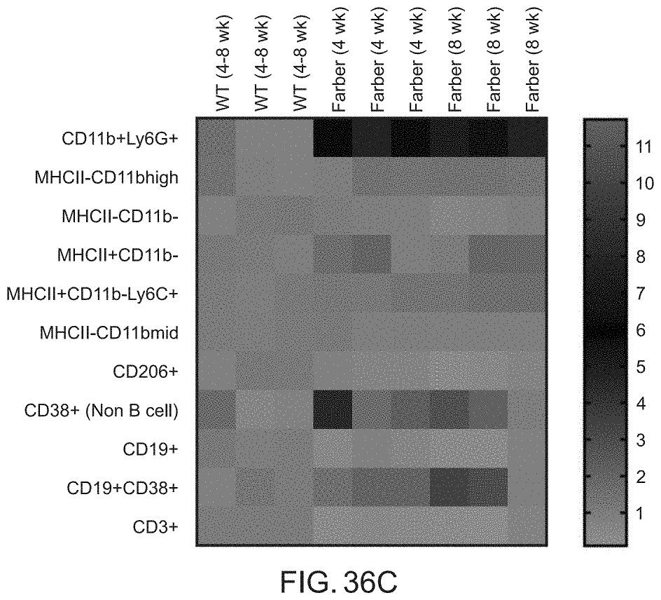

Systemic (blood) immune cell populations. Results are depicted in FIGS. 36A-C. Inflammatory cell populations characteristic of an inflammatory state were analyzed from control 4 and 8 week old wild-type and Farber mouse blood samples. The population of Ly6G, CD11b double positive CD45⁺neutrophils and CD11b⁺MHCII⁻CD45⁺ activated monocytes were determined from 7 week old Farber mice that were administered 10 mg/kg/dose RVT-801 once weekly beginning at 3 weeks of age for a total of 4 doses over 4 weeks. Heat map analysis of fold change differences in the frequency of immune cells in the blood of age-matched Farber mice and littermate controls (WT). Each column represents an individual animal. Fold change was calculated by setting the average WT value to 1.

### Example 5

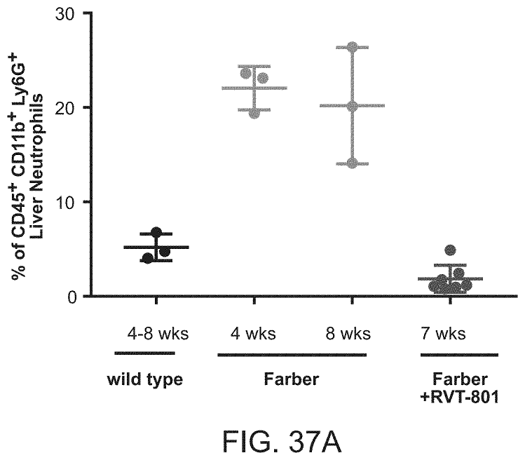

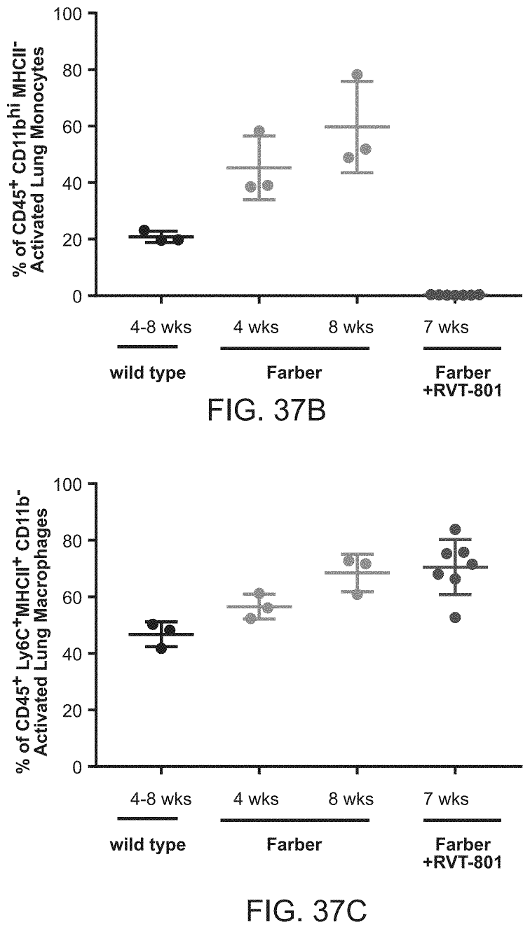

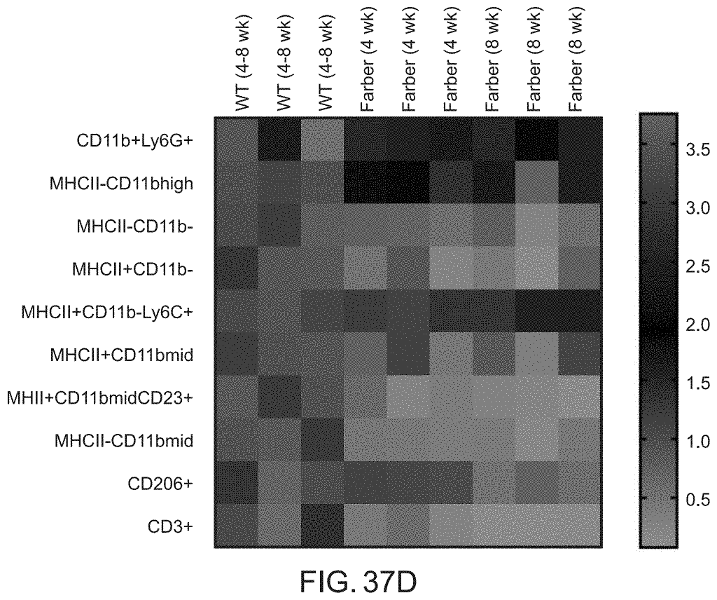

Pulmonary immune cell populations. Results are depicted in FIG. 37A-D. Inflammatory cell populations characteristic of an inflammatory state were analyzed from control 4 and 8 week old wild-type and Farber mouse lung tissue. The population of Ly6G, CD11b double positive CD45⁺neutrophils and CD11b⁺MHCII⁻CD45⁺ activated monocytes were determined from 7 week old Farber mice that were administered 10 mg/kg/dose RVT-801 once weekly beginning at 3 weeks of age for a total of 4 doses over 4 weeks. Heat map analysis of fold change differences in the frequency of immune cells in the lung of age-matched Farber mice and littermate controls (WT). Each column represents an individual animal. Fold change was calculated by setting the average WT value to 1. Also reported in FIG. 37C is an additional macrophage population that is CD45⁺Ly6C⁻MHCII⁺CD11b⁻.

### Example 6

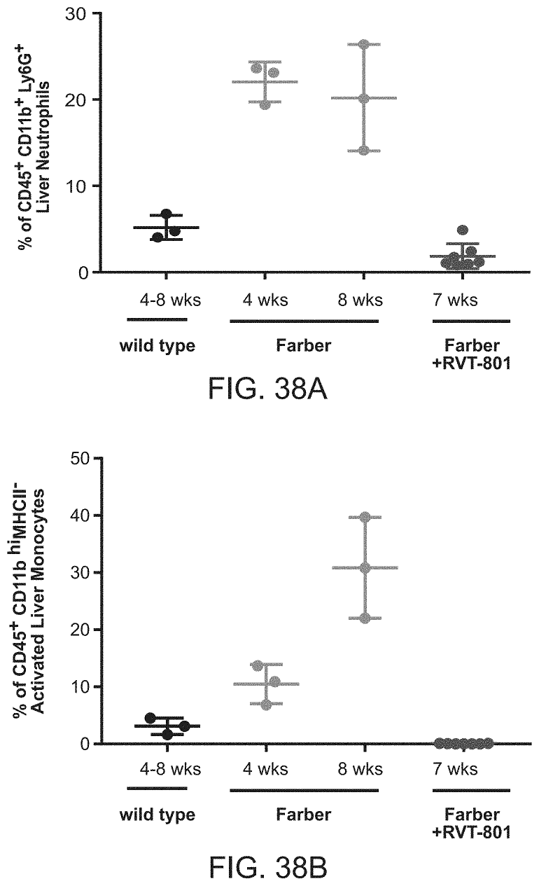

Hepatic immune cell populations. Results are depicted in FIGS. 38A-B. Inflammatory cell populations characteristic of an inflammatory state were analyzed from control 4 and 8 week old wild-type and Farber mouse liver tissue. The population of Ly6G/CD11b double positive CD45⁺ neutrophils and CD11b⁺hiMHCII⁻CD45⁺ activated monocytes were determined from 7 week old Farber mice that were administered 10 mg/kg/dose RVT-801 once weekly beginning at 3 weeks of age for a total of 4 doses over 4 weeks.

References discussed in the application, which are incorporated by reference in their entirety, for their intended purpose, which is clear based upon its context.
Alayoubi, A. M., J. C. Wang, B. C. Au, S. Carpentier, V. Garcia, S. Dworski, S. El-Ghamrasni, K. N. Kirouac, M. J. Exertier, Z. J. Xiong, G. G. Prive, C. M. Simonaro, J. Casas, G. Fabrias, E. H. Schuchman, P. V. Turner, R. Hakem, T. Levade, and J. A. Medin (2013). Systemic ceramide accumulation leads to severe and varied pathological consequences. EMBO Mol Med, 5:827-842.
Bae, J. S., Jang, K. H., Schuchman, E. H., and Jin, H. K. (2004). Comparative effects of recombinant acid sphingomyelinase administration by different routes in Niemann-Pick disease mice. Exp Anim, 53:417-421.
Becker, K. A., Riethmüller, J., Lüth, A., Döring, G., Kleuser, B., and Gulbins, E. (2010). Acid Sphingomyelinase Inhibitors Normalize Pulmonary Ceramide and Inflammation in Cystic Fibrosis. Am. J. Respir. Cell. Mol. Biol., 42(6):716-724.
Bernardo, K., R. Hurwitz, T. Zenk, R. J. Desnick, K. Ferlinz, E. H. Schuchman, and K. Sandhoff (1995). Purification, characterization, and biosynthesis of human acid ceramidase. J Biol Chem, 270:11098-11102.
Boado, R. J., Lu, J. Z., Hui, E. K., Lin, H., and Pardridge, W. M. (2016). Insulin receptor antibody-alpha-N-acetylglucosaminidase fusion protein penetrates the primate blood-brain barrier and reduces glycosaminoglycans in Sanfillippo type B fibroblasts. Mol. Pharm., 13:1385-92.
Chatelut, M., Harzer, K., Christomanou, H., Feunteun, J., Pieraggi, M. T., Paton, B. C., Kishimoto, Y., O'Brien, J. S., Basile, J. P., Thiers, J. C., Salvayre, R., and Levade, T. (1997). Model SV40-transformed fibroblast lines for metabolic studies of human prosaposin and acid ceramidase deficiencies. Clin Chim Acta, 262:61-76.
Desnick, R. J. and Schuchman, E. H. (2012). Enzyme replacement therapy for lysosomal storage diseases: lessons from 20 years of experience and remaining challenges. Annu Rev Genomics Hum Genet, 13:307-335.
Dworski, S., Berger, A., Furlonger, C., Moreau, J. M., Yoshimitsu, M., Trentadue, J., Au, B. C., Paige, C. J., and Medin, J. A. (2015). Markedly perturbed hematopoiesis in acid ceramidase deficient mice. Haematologica, 100(5):e162-165.
Dworski, S., Lu, P., Khan, A., Maranda, B., Mitchell, J. J., Parini, R., Di Rocco, M., Hugle, B., Yoshimitsu, M., Magnusson, B., Makay, B., Arslan, N., Guelbert, N., Ehlert, K., Jarisch, A., Gardner-Medwin, J., Dagher, R., Terreri, M. T., Lorenco, C. M., Barillas-Arias, L., Tanpaiboon, P., Solyom, A., Norris, J. S., He, X., Schuchman, E. H., Levade, T., and Medin, J. A. (2017). Acid Ceramidase Deficiency is characterized by a unique plasma cytokine and ceramide profile that is altered by therapy. Biochim Biophys Acta, 1863(2):386-394 (Epub December 2016 doi: 10.1016/j.bbadis.2016.11.031).
Eliyahu, E., Park, J. H., Shtraizent, N., He, X., and Schuchman, E. H. (2007). Acid ceramidase is a novel factor required for early embryo survival. FASEB J., 21:1403-9.
Eliyahu, E., N. Shtraizent, K. Martinuzzi, J. Barritt, X. He, H. Wei, S. Chaubal, A. B. Copperman, and E. H. Schuchman (2010). Acid ceramidase improves the quality of oocytes and embryos and the outcome of in vitro fertilization. FASEB J, 24:1229-1238.
Eliyahu, E., N. Shtraizent, R. Shalgi and E. H. Schuchman (2012). Construction of conditional acid ceramidase knockout mice and in vivo effects on oocyte development and fertility. Cellular physiology and biochemistry: international journal of experimental cellular physiology, biochemistry, and pharmacology, 30:735-748.
Farber, S. (1952) A lipid metabolic disorder—disseminated “Lipogranulomatosis”—a syndrome with similarity to, and important difference from, Niemann-Pick and Hand-Schuller-Christian disease. Am. J. Dis. Child, 84:499.
Frohbergh, M E., Guevara, J. M., Greisamer, R. P., Barbe, M. F., He, X., Simonaro, C. M., and Schuchman, E. H. (2016). Acid ceramidase treatment enhances the outcome of autologous chondrocyte implantation in a rat osteochondral defect model. Osteoarthritis Cartilage, 24:752-762.
Gatt, S. (1963). Enzymic hydrolysis and synthesis of ceramides. J Biol Chem, 238:3131-3133.
He, X., N. Okino, R. Dhami, A. Dagan, S. Gatt, H. Schulze, K. Sandhoff, and E. H. Schuchman (2003). Purification and characterization of recombinant, human acid ceramidase. Catalytic reactions and interactions with acid sphingomyelinase. J Biol Chem, 278(35):32978-32986.
He et al. (2017). Enzyme replacement therapy for Farber disease: Proof-of-concept studies in cells and mice,” BBA Clin. 2017 Feb. 13; 7:85-96.
Hollak, C. E. and Wijburg, F. A. (2014). Treatment of lysosomal storage disorders: successes and challenges. J Inherit Metab Dis, 37:587-598.
Jablonski, K. A., Amici, S. A., Webb, L. M., Ruiz-Rosado, Jd. D., Popovich, P. G., Partida-Sanchez, S., and Guerau-de-Arellano, M. (2015). Novel Markers to Delineate Murine M1 and M2 Macrophages. PLoS ONE 10(12):e0145342.
Koch, J., S. Gartner, C. M. Li, L. E. Quintern, K. Bernardo, O. Levran, D. Schnabel, R. J. Desnick, E. H. Schuchman, and K. Sandhoff (1996). Molecular cloning and characterization of a full-length complementary DNA encoding human acid ceramidase. Identification of the first molecular lesion causing Farber disease. J Biol Chem, 271(51):33110-33115.
Jun Kunisawa, Yosuke Kurashima, Morio Higuchi, Masashi Gohda, Izumi Ishikawa, Ikuko Ogahara, Namju Kim, Miki Shimizu, and Hiroshi Kiyono (2007). Sphingosine 1-phosphate dependence in the regulation of lymphocyte trafficking to the gut epithelium, JEM, 204 (10): 2335-2348.
Li, C M., J. H. Park, X. He, B. Levy, F. Chen, K. Arai, D A. Adler, C M. Disteche, J. Koch, K. Sandhoff, and E. H. Schuchman (1999). The human acid ceramidase gene (asah): Structure, chromosomal location, mutation analysis, and expression. Genomics, 62(2):223-231.
Li, C M., S B. Hong, G. Kopal, X. He, T. Linke, W. S. Hou, J. Koch, S. Gatt, K. Sandhoff, and E. H. Schuchman (1998). Cloning and characterization of the full-length cDNA and genomic sequences encoding murine acid ceramidase. Genomics, 50(2):267-274.
Li, C M., J. H. Park, C M. Simonaro, X. He, R E. Gordon, A H. Friedman, D. Ehleiter, F. Paris, K. Manova, S. Hepbildikler, Z. Fuks, K. Sandhoff, R. Kolesnick, and E. H. Schuchman (2002). Insertional mutagenesis of the mouse acid ceramidase gene leads to early embryonic lethality in homozygotes and progressive lipid storage disease in heterozygotes. Genomics, 79(2):218-224.
Misharin A V, Morales-Nebreda L, Mutlu G M, Budinger G R S, and Perlman H (2013). Flow Cytometric Analysis of Macrophages and Dendritic Cell Subsets in the Mouse Lung, Am J Respir Cell Mol Biol, 49 (4): 503-510.
Murray J M, Thompson, A M, Vitsky A, Hawes M, Chuang W L, Pacheco J, Wilson S, McPherson J M, Thurberg B L, Karey K P, and Andrews L. (2015). Nonclinical safety assessment of recombinant human acid sphingomyelinase (rhASM) for the treatment of acid sphingomyelinase deficiency: the utility of animal models of disease in the toxicology evaluation of potential therapeutics. Mol Genet Metab, 114:217-225.
Okino, N., He, X., S. Gatt, K. Sandhoff, M. Ito, and E. H. Schuchman (2003). The reverse activity of human acid ceramidase. J Biol Chem, 278(32):29948-29953.
Realini, N., Palese, F., Pizzirani, D., Pontis, S., Basit, A., Bach, A., Ganesan, A., and Piomelli, D. (2015). Acid ceramidase in melanoma: expression, localization and effects of pharmacological inhibition. J Biol Chem, N291:2422-2434.
Roh, J. L., Park, J. Y., Kim, E. H., and Jang, H. J. (2016). Targeting acid ceramidase sensitises head and neck cancer to cisplatin. Eur J Cancer, 52:163-72.
Shiffmann, S., Hartmann, D., Birod, K., Ferreiròs, N., Schreiber, Y., Zivkovic, A., Geisslinger, G., Grosch, S., and Stark, H. (2012). Inhibitors of Specific Ceramide Synthases. Biochimie, 94(2):558-565.
Schuchman, E. H. (2016). Acid ceramidase and the treatment of ceramide diseases. The expanding role of enzyme replacement therapy. Biochim Bipphys Acta, 1862:1459-1471.
Simonaro, C. M., Sachot, S., Ge, Y., He, X., DeAngelis, V. A., Eliyahu, E., Leong, D. J., Sun, H. B., Mason, J. B., Haskins, M. E., Richardson, D. W., and Schuchman, E. H. (2013). Acid ceramidase maintains the chondrogenic phenotype of expanded primary chondrocytes and improves the chondrogenic differentiation of bone marrow-derived mesenchymal stem cells. PLoS One, 8:e62715.
Shtraizent, N., E. Eliyahu, J. H. Park, X. He, R. Shalgi and E. H. Schuchman (2008). Autoproteolytic cleavage and activation of human acid ceramidase. J Biol Chem, 283(17): 11253-11259.
Sugita, M., Dulaney, J. T., and Moser, H W (1972). Ceramidase deficiency in Farber's disease (lipogranulomatosis). Science, 178(4065): 1100-1102.
Yu F P, Islam D, Sikora J, Dworski S, Gurka'J, Lopez-Vasquez L, Liu M, Kuebler W M, Levade T, Zhang H, Medin J A (2017). Chronic lung injury and impaired pulmonary function in a mouse model of acid ceramidase deficiency, Press. Am J Physiol Lung Cell Mol Physiol (Nov. 22, 2017, Epub ahead of print).

The disclosures of each and every patent, patent application, publication, and accession number cited herein are hereby incorporated herein by reference in their entirety.

While present disclosure has been disclosed with reference to various embodiments, it is apparent that other embodiments and variations of these may be devised by others skilled in the art without departing from the true spirit and scope of the disclosure. The appended claims are intended to be construed to include all such embodiments and equivalent variations.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A method for determining whether a subject has Farber disease, the method comprising detecting the level of at least one marker selected from CD11b⁺Ly6G⁺, SSC^{mid} FSC^{mid}, MHCII⁻CD11b^{hi}, MHCII⁺CD11b⁻Ly6C⁺, MHCII⁻CD11b^{hi}CD86⁺, CD11b⁺CD38⁺, CD19⁺CD38⁺, CD11b⁺CD206⁺, MHCII⁺CD11b^{mid}CD23⁺, and CD19⁻ CD3⁺ in a biological sample from a subject, wherein
if the level of CD11b⁺Ly6G⁺, SSC^{mid} FSC^{mid}, MHCII⁻CD11b^{hi}, MHCII⁺CD11b⁻Ly6C⁺, MHCII⁻CD11b^{hi}CD86⁺, CD11b⁺CD38⁺, CD19⁺CD38⁺ is higher than a control, the subject has Farber disease; and
if the level of CD11b⁺CD206⁺, MHCII⁺CD11b^{mid}CD23⁺, and CD19⁻CD3⁺ is lower than a control, the subject has Farber disease.

2. The method of claim 1, further comprising detecting the level of MHCII⁺CD11b⁻Ly6C⁺, in a sample from the subject, wherein a level of MHCII⁺CD11b⁻Ly6C⁺ that is higher than a control level indicates that the subject has Farber disease.

3. The method according to claim 1, further comprising detecting the level of MHCII⁻CD11b^{hi}CD86⁺, in a sample from the subject, wherein a level of MHCII⁻CD11b^{hi}CD86⁺ that is higher than a control level indicates that the subject has Farber disease.

4. The method according to claim 1, further comprising detecting the level of CD11b⁺CD38⁺, in a sample from the subject, wherein a level of CD11b⁺CD38⁺ that is higher than a control level indicates that the subject has Farber disease.

5. The method according to claim 1, further comprising detecting the level of CD11b⁺CD206⁺, in a sample from the subject, wherein a level of CD11b⁺CD206⁺ that is lower than a control level indicates that the subject has Farber disease.

6. The method according to claim 1, further comprising detecting the level of CD11b⁺Ly6G⁺, in a sample from the subject, wherein a level of CD11b⁺Ly6G⁺ that is higher than a control level indicates that the subject has Farber disease.

7. The method according to claim 1, further comprising detecting the level of CD19⁺CD38⁺, in a sample from the subject, wherein a level of CD19⁺CD38⁺ that is higher than a control level indicates that the subject has Farber disease.

8. The method according to claim 1, further comprising detecting the level of CD19⁻CD3⁺, in a sample from the subject, wherein a level of CD19⁻CD3⁺ that is lower than a control level indicates that the subject has Farber disease.

9. The method according to claim 1, where the detection is performed by detecting the levels of MHCII⁺CD11b⁻Ly6C⁺ and MHCII⁻CD11b^{hi}CD86⁺ in a sample from the subject, wherein a level of MHCFII⁺CD11b⁻Ly6C⁺ and/or MHCII⁻CD11b^{hi}CD86⁺ that is higher than a control level indicates that the subject has Farber disease.

10. The method according to claim 1, wherein the detection is performed by detecting the level of CD19⁺CD38⁺ in a sample from the subject, and further detecting a level of CD19⁻CD3⁺ in a sample from the subject, wherein a level of CD19⁺CD38⁺ that is higher than a control level and/or a level of CD19⁻CD3⁺ lower than a control level, and the combined detection indicates that the subject has Farber disease.

11. The method according to claim 1, wherein the detection is performed by detecting the levels of at least four, at least five, at least six, at least seven, at least eight, at least nine, or ten markers, selected from CD11b⁺Ly6G⁺, SSC^{mid} FSC^{mid}, MHCII⁻CD11b^{hi}, MHCII⁺CD11b⁻Ly6C⁺, MHCII⁻CD11b^{hi}CD86⁺, CD11b⁺CD38⁺, CD19⁺CD38⁺, CD11b⁺CD206⁺, MHCII⁺CD11b^{mid}CD23⁺, and CD19⁻CD3⁺ to determine whether a subject has Farber disease.

12. The method of claim 1, wherein the biological sample is a tissue extract sample or a blood sample.

13. The method of claim 12, wherein the biological sample is obtained from liver, spleen, lung, or blood.

14. The method of claim 1, further comprising administering a therapeutically effective amount of a pharmaceutical composition useful in the treatment of Farber disease.

15. The method of claim 14, wherein the composition comprises a recombinant human acid ceramidase (rhAC).

16. The method of claim 15, wherein the rhAC is in an amount of about 0.1 mg/kg to about 50 mg/kg.

17. A kit for performing the method of claim 1 together with instructions for use in diagnosing Farber disease.

18. The kit of claim 17, wherein the kit comprises at least one antibody that specifically binds marker CD11b⁺Ly6G⁺, SSC^{mid} FSC^{mid}, MHCII⁻CD11b^{hi}, MHCII⁺CD11b⁻Ly6C⁺, MHCII⁻CD11b^{hi}CD86⁺, CD11b⁺CD38⁺, CD19⁺CD38⁺, CD11b⁺CD206⁺, MHCII⁺CD11b^{mid}CD23⁺, or CD19⁻CD3⁺.

19. A method for treating Farber disease, the method comprising:
detecting a level of at least one marker selected from CD11b⁺Ly6G⁺, SSC^{mid}FSC^{mid}, MHCII⁻CD11b^{hi}, MHCII⁺CD11b⁻Ly6C⁺, MHCII⁻CD11b^{hi}CD86⁺, CD11b⁺CD38⁺, CD19⁺CD38⁺, CD11b⁺CD206⁺, MHCII⁺CD11b^{mid}CD23⁺, and CD19⁻CD3⁺ in a sample from a subject, wherein
if the level of CD11b⁺Ly6G⁺, SSC^{mid} FSC^{mid}, MHCII⁻CD11b^{hi}, MHCII⁺CD11b⁻Ly6C⁺, MHCII⁻CD11b^{hi}CD86⁺, CD11b⁺CD38⁺, CD19⁺CD38⁺ is higher than a control, the subject has Farber disease; and
if the level of CD11b⁺CD206⁺, MHCII⁺CD11b^{mid}CD23⁺, and CD19⁻CD3⁺ is lower than a control, the subject has Farber disease; and
administering a therapeutically effective amount of a pharmaceutical composition useful in the treatment of Farber disease.

20. The method according to claim 19, wherein the pharmaceutical composition comprises a recombinant human acid ceramidase (rhAC).

21. The method of claim 20, wherein the rhAC in an amount of about 0.1 mg/kg to about 50 mg/kg.


SEQUENCE LISTING (4 sequences)

SEQ ID NO: 1 - protein, 395 aa, Artificial Sequence
  Description of Artificial Sequence Synthetic polypeptide
  Recombinant human acid ceramidase
MPGRSCVALVLLAAAVSCAVAQHAPPWTEDCRKSTYPPSGPTYRGAVPWYTINLDLPPYKRWHELMLDKAPVLKVIVNSLKNMINTFVPSGKIMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 2 - DNA, 2618 bp, Artificial Sequence
  Description of Artificial Sequence Synthetic polynucleotide
  Encodes recombinant human acid ceramidase
ggctcggtccgactattgcccgcggtgggggagggggatggatcacgccacgcgccaaaggcgatcgcgactctccttctgcaggtagcctggaaggctctctctctttctctacgccacccttttcgtggcactgaaaagccccgtcctctcctcccagtcccgcctcctccgagcgttccccctactgcctggaatggtgcggtcccaggtcgcgggtcacgcggcggagggggcgtggcctgcccccggcccagccggctcttctttgcctctgctggagtccggggagtggcgttggctgctagagcgatgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtgagcacacgtctggcctacagaatgcggcctctgagacatgaagacaccatctccatgtgaccgaacactgcagctgtctgaccttccaaagactaagactcgcggcaggttctctttgagtcaatagcttgtcttcgtccatctgttgacaaatgacagatcttttttttttccccctatcagttgatttttcttatttacagataacttctttaggggaagtaaaacagtcatctagaattcactgagttttgtttcactttgacatttggggatctggtgggcagtcgaaccatggtgaactccacctccgtggaataaatggagattcagcgtgggtgttgaatccagcacgtctgtgtgagtaacgggacagtaaacactccacattcttcagtttttcacttctacctacatatttgtatgtttttctgtataacagccttttccttctggttctaactgctgttaaaattaatatatcattatctttgctgttattgacagcgatataattttattacatatgattagagggatgagacagacattcacctgtatatttcttttaatgggcacaaaatgggcccttgcctctaaatagcactttttggggttcaagaagtaatcagtatgcaaagcaatcttttatacaataattgaagtgttccctttttcataattactctacttcccagtaaccctaaggaagttgctaacttaaaaaactgcatcccacgttctgttaatttagtaaataaacaagtcaaagacttgtggaaaataggaagtgaacccatattttaaattctcataagtagcattcatgtaataaacaggtttttagtttgttcttcagattgatagggagttttaaagaaattttagtagttactaaaattatgttactgtatttttcagaaatcaaactgcttatgaaaagtactaatagaacttgttaacctttctaaccttcacgattaactgtgaaatgtacgtcatttgtgcaagaccgtttgtccacttcattttgtataatcacagttgtgttcctgacactcaataaacagtcactggaaagagtgccagtcagcagtcatgcacgctgattgggtgtgt

SEQ ID NO: 3 - DNA, 1276 bp, Artificial Sequence
  Description of Artificial Sequence Synthetic polynucleotide
  Encodes recombinant human acid ceramidase
aagcttaccgccaccatgaactgctgcatcggcctgggtgagaaggcgcgtggctcgcaccgcgccagctacccctccctgagcgccctcttcaccgaggcgtccatcctcggattcgggagcttcgccgtcaaggcacagtggaccgaggattgccgcaagagtacgtacccccccagtggcccgacgtaccgcggcgccgtcccctggtacacgatcaacctggacctccccccgtacaagcgctggcacgagttgatgctggacaaggcccccgtactgaaggtcatcgtgaactccctgaagaacatgatcaacaccttcgtcccctcgggcaagatcatgcaggtcgtggacgagaagctgcccgggctcctcggcaacttccccggcccgttcgaagaggagatgaagggcatcgcggccgtcactgacatccccctgggcgagatcatcagcttcaacatcttctacgagctgttcaccatctgcacctccatcgtagccgaggacaagaagggccacctgatccacggtcgcaacatggacttcggcgtcttcctgggctggaacatcaacaacgacacctgggtcatcaccgagcagctgaagccgctcaccgtgaacctcgatttccagcgcaacaacaagacggtgttcaaggccagctccttcgccgggtacgtcgggatgctcacgggcttcaagccgggactgttctcgctgaccctcaacgagcggttctccatcaacgggggctacctcggcatcctggagtggattctcggcaagaaggacgtgatgtggatcggcttcctcacacggaccgtgctggaaaactccactagttacgaggaggccaagaacctgctgaccaagacgaagatcctggccccggcatacttcatcctgggcggcaaccagtcgggcgaggggtgcgtcatcacccgcgaccggaaggagtccctggacgtctatgagctggacgccaagcagggccgctggtacgtcgtccagacgaactacgaccgatggaagcaccccttcttcctcgacgaccggcgcacgcccgccaagatgtgcctgaaccgcaccagccaggagaacatctcgttcgagacgatgtacgacgtgctgtcgaccaagcccgtgctcaacaagctgacggtctacaccacgctgatcgacgtgacgaaaggccagttcgaaacgtacctgcgggactgcccggacccttgcatcggctggtgataatctagagtcggggcggccggcc

SEQ ID NO: 4 - DNA, 1276 bp, Artificial Sequence
  Description of Artificial Sequence Synthetic polynucleotide
  Encodes recombinant human acid ceramidase
aagcttaccgccaccatgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttccagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtgataacctagggtcggggcggccggcc